SEQUENCE LISTING (32 sequences)

SEQ ID NO: 1 - protein, 1118 aa, Homo sapiens
MARSPGRAYALLLLLICFNVGSGLHLQVLSTRNENKLLPKHPHLVRQKRAWITAPVALREGEDLSKKNPIAKIHSDLAEERGLKITYKYTGKGITEPPFGIFVFNKDTGELNVTSILDREETPFFLLTGYALDARGNNVEKPLELRIKVLDINDNEPVFTQDVFVGSVEELSAAHTLVMKINATDADEPNTLNSKISYRIVSLEPAYPPVFYLNKDTGEIYTTSVTLDREEHSSYTLTVEARDGNGEVTDKPVKQAQVQIRILDVNDNIPVVENKVLEGMVEENQVNVEVTRIKVFDADEIGSDNWLANFTFASGNEGGYFHIETDAQTNEGIVTLIKEVDYEEMKNLDFSVIVANKAAFHKSIRSKYKPTPIPIKVKVKNVKEGIHFKSSVISIYVSESMDRSSKGQIIGNFQAFDEDTGLPAHARYVKLEDRDNWISVDSVTSEIKLAKLPDFESRYVQNGTYTVKIVAISEDYPRKTITGTVLINVEDINDNCPTLIEPVQTICHDAEYVNVTAEDLDGHPNSGPFSFSVIDKPPGMAEKWKIARQESTSVLLQQSEKKLGRSEIQFLISDNQGFSCPEKQVLTLTVCECLHGSGCREAQHDSYVGLGPAAIALMILAFLLLLLVPLLLLMCHCGKGAKGFTPIPGTIEMLHPWNNEGAPPEDKVVPSFLPVDQGGSLVGRNGVGGMAKEATMKGSSSASIVKGQHEMSEMDGRWEEHRSLLSGRATQFTGATGAIMTTETTKTARATGASRDMAGAQAAAVALNEEFLRNYFTDKAASYTEEDENHTAKDCLLVYSQEETESLNASIGCCSFIEGELDDRFLDDLGLKFKTLAEVCLGQKIDINKEIEQRQKPATETSMNTASHSLCEQTMVNSENTYSSGSSFPVPKSLQEANAEKVTQEIVTERSVSSRQAQKVATPLPDPMASRNVIATETSYVTGSTMPPTTVILGPSQPQSLIVTERVYAPASTLVDQPYANEGTVVVTERVIQPHGGGSNPLEGTQHLQDVPYVMVRERESFLAPSSGVQPTLAMPNIAVGQNVTVTERVLAPASTLQSSYQIPTENSMTARNTTVSGAGVPGPLPDFGLEESGHSNSTITTSSTRVTKHSTVQHSYS

SEQ ID NO: 2 - DNA, 5697 bp, Homo sapiens
  source 1..5697: mol_type = genomic DNA
gaggagccgagtgcgcgctcggggcaggcggcggcgcggagcggtgcggcggcgggaggcggaggcgagggtgcgatggcgcggagcccgggacgcgcgtacgccctgctgcttctcctgatctgctttaacgttggaagtggacttcacttacaggtcttaagcacaagaaatgaaaataagctgcttcctaaacatcctcatttagtgcggcaaaagcgcgcctggatcaccgcccccgtggctcttcgggagggagaggatctgtccaagaagaatccaattgccaagatacattctgatcttgcagaagaaagaggactcaaaattacttacaaatacactggaaaagggattacagagccaccttttggtatatttgtctttaacaaagatactggagaactgaatgttaccagcattcttgatcgagaagaaacaccattttttctgctaacaggttacgctttggatgcaagaggaaacaatgtagagaaacccttagagctacgcattaaggttcttgatatcaatgacaacgaaccagtgttcacacaggatgtctttgttgggtctgttgaagagttgagtgcagcacatactcttgtgatgaaaatcaatgcaacagatgcagatgagcccaataccctgaattcgaaaatttcctatagaatcgtatctctggagcctgcttatcctccagtgttctacctaaataaagatacaggagagatttatacaaccagtgttaccttggacagagaggaacacagcagctacactttgacagtagaagcaagagatggcaatggagaagttacagacaaacctgtaaaacaagctcaagttcagattcgtattttggatgtcaatgacaatatacctgtagtagaaaataaagtgcttgaagggatggttgaagaaaatcaagtcaacgtagaagttacgcgcataaaagtgttcgatgcagatgaaataggttctgataattggctggcaaattttacatttgcatcaggaaatgaaggaggttatttccacatagaaacagatgctcaaactaacgaaggaattgtgacccttattaaggaagtagattatgaagaaatgaagaatcttgacttcagtgttattgtcgctaataaagcagcttttcacaagtcgattaggagtaaatacaagcctacacccattcccatcaaggtcaaagtgaaaaatgtgaaagaaggcattcattttaaaagcagcgtcatctcaatttatgttagcgagagcatggatagatcaagcaaaggccaaataattggaaattttcaagcttttgatgaggacactggactaccagcccatgcaagatatgtaaaattagaagatagagataattggatctctgtggattctgtcacatctgaaattaaacttgcaaaacttcctgattttgaatctagatatgttcaaaatggcacatacactgtaaagattgtggccatatcagaagattatcctagaaaaaccatcactggcacagtccttatcaatgttgaagacatcaacgacaactgtcccacactgatagagcctgtgcagacaatctgtcacgatgcagagtatgtgaatgttactgcagaggacctggatggacacccaaacagtggccctttcagtttctccgtcattgacaaaccacctggcatggcagaaaaatggaaaatagcacgccaagaaagtaccagtgtgctgctgcaacaaagtgagaaaaagcttgggagaagtgaaattcagttcctgatttcagacaatcagggttttagttgtcctgaaaagcaggtccttacactcacagtttgtgagtgtctgcatggcagcggctgcagggaagcacagcatgactcctatgtgggcctgggacccgcagcaattgcgctcatgattttggcctttctgctcctgctattggtaccacttttactgctgatgtgccattgcggaaagggcgccaaaggctttacccccatacctggcaccatagagatgctgcatccttggaataatgaaggagcaccacctgaagacaaggtggtgccatcatttctgccagtggatcaagggggcagtctagtaggaagaaatggagtaggaggtatggccaaggaagccacgatgaaaggaagtagctctgcttccattgtcaaagggcaacatgagatgtccgagatggatggaaggtgggaagaacacagaagcctgctttctggtagagctacccagtttacaggggccacaggcgctatcatgaccactgaaaccacgaagaccgcaagggccacaggggcttccagagacatggccggagctcaggcagctgctgttgcactgaacgaagaattcttaagaaattatttcactgataaagcggcctcttacactgaggaagatgaaaatcacacagccaaagattgccttctggtttattctcaggaagaaactgaatcgctgaatgcttctattggttgttgcagttttattgaaggagagctagatgaccgcttcttagatgatttgggacttaaattcaagacactagctgaagtttgcctgggtcaaaaaatagatataaataaggaaattgagcagagacaaaaacctgccacagaaacaagtatgaacacagcttcacattcactctgtgagcaaactatggttaattcagagaatacctactcctctggcagtagcttcccagttccaaaatctttgcaagaagccaatgcagagaaagtaactcaggaaatagtcactgaaagatctgtgtcttctaggcaggcgcaaaaggtagctacacctcttcctgacccaatggcttctagaaatgtgatagcaacagaaacttcctatgtcacagggtccactatgccaccaaccactgtgatcctgggtcctagccagccacagagccttattgtgacagagagggtgtatgctccagcttctaccttggtagatcagccttatgctaatgaaggtacagttgtggtcactgaaagagtaatacagcctcatgggggtggatcgaatcctctggaaggcactcagcatcttcaagatgtaccttacgtcatggtgagggaaagagagagcttccttgcccccagctcaggtgtgcagcctactctggccatgcctaatatagcagtaggacagaatgtgacagtgacagaaagagttctagcacctgcttccactctgcaatccagttaccagattcccactgaaaattctatgacggctaggaacaccacggtgtctggagctggagtccctggccctctgccagattttggtttagaggaatctggtcattctaattctaccataaccacatcttccaccagagttaccaagcatagcactgtacagcattcttactcctaaacagcagtcagccacaaactgacccagagtttaattagcagtgactaatttcatgtttccaatgtacctgatttttcatgagccttacagacacacagagacacatacacattgatcttaaaatttttctcagtcactgatatgcaaaggaccacactgtctctgcttccaggagtattttagaaatgttccacaatttactgaagacatagagatgatgctgctgcttaggtgccttttagcaagctatgcaaacaatcctgataaaacaagatacatagagagtcaatctggcttctgagaatttaccaagtgaacagagtacctagttcatcagccgtccagtaaagcaacccaggaaactgactgggtctctttgcctaccgtattaacattaaacattgatgttctgtattctgtactttactgcacccagcagactttcaacaactcattgatccaaagatacatgcacagtctgagcaccagctatggtgctcataacttctttaagacttgaaccctttcaatctgtgtgattcattaaattggaccattgatgataagaatacacattgtatgtttctgtgcacatgacagtgtgtgtgtgtgcacgtacatactgtatagtcttaaaaatagcattatactggccaggggtggtggctaacgcctgtaatcccagcactttgggaggccgaggcgggtggatcaactgtggtcaggagtttgagatcagccaggccaacctggtgaaaccccgtctctactaaaaatacaaaaattagctgggcgtgatggtgggcgcctgtaatcccagctacttgggaggctgaggcaggagaatcacttgaacccgggaggcggaggttgcagtgagccgagatcgcaccattgcactccagtctgggcaacagagtgagattccgtctcaaaaaaaaaaagaaaaggaaaaaaaaatagcattatacctcttccttgtctcaaccgccatgaaaattctgaacactccaaattcagttgaataatccaaaacaaaatttataagtataaaataattttacttcttatagtaatagtatactttaaaaagcctcagggtatattatcttctaaacagctacaattcagtgcagctacattaaccaactatgttctctagttgagaacaactaggcctatttcactgctgtgtagcctcagtgcctaacatgggtgccaaataaatattcgtagaattacactgaattgtaaaaaccattcgtttttgtttacaattgccaaaaatctcaaaaggccctgtatttatgtaattctttgaaattattattttattttgatttctcagttattgactggctgggtgtgacttagtacataagtactcaatattataaaaacctcaaataattgacttgattttacacaacatccttcccttttctacaagttaatttttttacaaatcatttgggttatctcctaaataggttatattttattgcttctagaaacaatgtttcaaaatatatgtgcattatcagtaataatttgtataaatatttcccacaacaattttcataattttcaaagactaatttcttgactgaagatattttgctagggaagtgaaactttaaaattttgtagattttaaaaaatattgttgaatggtgtcatgcaaaggatttatatagtgtgctcccactaactgtacagatcaggacacatatttttagacatctaagtctgtagcttaaatggaggttactcttccatcatctagaattgtttacttagtaattgttgtttcttttattattatagacttactatcagttttattttgccaagtatgcaacaggtatatcactagtatatgaaaatgtaaatatcacttgtgtactcaaacaaaagttggtcttaagcttccaccttgagcagccttggaaacctaacctgcctcttttagcataatcacattttctaaatgattttctttgttcctgaaaaagtgatttgtattagttttacatttgttttttggaagattatatttgtatatgtatcatcataaaatatttaaataaaaagtatctttagagtgaccctttccccatagatttttatttctctattatattttacaaggaatataactcagtttgttagggagagtgccttaaaggcaggtgtttcttggactttgttatttaattagatctgcttgcaataaaaaaagttgtcggttatctaaaattcaaa

SEQ ID NO: 3 - protein, 560 aa, Homo sapiens
AWITAPVALREGEDLSKKNPIAKIHSDLAEERGLKITYKYTGKGITEPPFGIFVFNKDTGELNVTSILDREETPFFLLTGYALDARGNNVEKPLELRIKVLDINDNEPVFTQDVFVGSVEELSAAHTLVMKINATDADEPNTLNSKISYRIVSLEPAYPPVFYLNKDTGEIYTTSVTLDREEHSSYTLTVEARDGNGEVTDKPVKQAQVQIRILDVNDNIPVVENKVLEGMVEENQVNVEVTRIKVFDADEIGSDNWLANFTFASGNEGGYFHIETDAQTNEGIVTLIKEVDYEEMKNLDFSVIVANKAAFHKSIRSKYKPTPIPIKVKVKNVKEGIHFKSSVISIYVSESMDRSSKGQIIGNFQAFDEDTGLPAHARYVKLEDRDNWISVDSVTSEIKLAKLPDFESRYVQNGTYTVKIVAISEDYPRKTITGTVLINVEDINDNCPTLIEPVQTICHDAEYVNVTAEDLDGHPNSGPFSFSVIDKPPGMAEKWKIARQESTSVLLQQSEKKLGRSEIQFLISDNQGFSCPEKQVLTLTVCECLHGSGCREAQHDSYVG

SEQ ID NO: 4 - protein, 330 aa, Homo sapiens [sequence omitted]

SEQ ID NO: 5 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 7 - protein, 228 aa, Homo sapiens
ERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 8 - protein, 377 aa, Homo sapiens
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 9 - protein, 279 aa, Homo sapiens
ELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 10 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 11 - protein, 229 aa, Homo sapiens
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 12 - protein, 6 aa, synthetic construct
GGGGGS

SEQ ID NO: 13 - protein, 5 aa, synthetic construct
EAAAK

SEQ ID NO: 14 - protein, 1122 aa, Mus musculus
MARSPGDRCALLLLVQLLAVVCLDFGNGLHLEVFSPRNEGKPFPKHTHLVRQKRAWITAPVALREGEDLSRKNPIAKIHSDLAEEKGIKITYKYTGKGITEPPFGIFVFDRNTGELNITSILDREETPYFLLTGYALDSRGNNLEKPLELRIKVLDINDNEPVFTQEVFVGSIEELSAAHTLVMKITATDADDPETLNAKVSYRIVSQEPANSHMFYLNKDTGEIYTTSFTLDREEHSSYSLTVEARDGNGQITDKPVQQAQVQIRILDVNDNIPVVENKMYEGTVEENQVNVEVMRIKVTDADEVGSDNWLANFTFASGNEGGYFHIETDTQTNEGIVTLVKEVDYEEMKKLDLSIIVTNKAAFHKSILSKYKATPIPITVKVKNVVEGIHFKSSVVSFRASEAMDRSSLSRSIGNFQVFDEDTGQAAKVTYVKVQDTDNWVSVDSVTSEIKLVKIPDFESRYVQNGTYTAKVVAISKEHPQKTITGTIVITVEDVNDNCPVLVDSVRSVCEDEPYVNVTAEDLDGAQNSAPFSFSIIDQPPGTAQKWKITHQESTSVLLQQSERKRGRSEIPFLISDSQGFSCPERQVLQLTVCECLKGGGCVAAQYDNYVGLGPAAIALMILALLLLLLVPLLLLICHCGGGAKGFTPIPGTIEMLHPWNNEGAPPEDKVVPSLLVADHAESSAVRGGVGGAMLKEGMMKGSSSASVTKGQHELSEVDGRWEEHRSLLTAGATHHVRTAGTIAANEAVRTRATGSSRDMSGARGAVAVNEEFLRSYFTEKAASYNGEDDLHMAKDCLLVYSQEDTASLRGSVGCCSFIEGELDDLFLDDLGLKFKTLAEVCLGRKIDLDVDIEQRQKPVREASVSAASGSHYEQAVTSSESAYSSNTGFPAPKPLHEVHTEKVTQEIVTESSVSSRQSQKVVPPPDPVASGNIIVTETSYAKGSAVPPSTVLLAPRQPQSLIVTERVYAPTSTLVDQHYANEEKVLVTERVIQPNGGIPKPLEVTQHLKDAQYVMVRERESILAPSSGVQPTLAMPSVAAGGQNVTVTERILTPASTLQSSYQIPSETSITARNTVLSSVGSIGPLPNLDLEESDRPNSTITTSSTRVTKHSTMQHSYS

SEQ ID NO: 15 - protein, 1128 aa, Rattus norvegicus
MARSPSGRCTLLLLLQLLAVVCLDFGDGLHLEVFSTRGEDRLFTKRTHLVRQKRAWITAPVALREGEDLSKKNPIAKIHSDLAEERGIKITYKYTGKGITEPPFGIFVFDRNTGDLNITSILDREETPFFLLTGYALDSRGNNLEKPLELRIKVLDINDNEPVFTQEVFVGSIEELSAAHTLVMKITATDADDPQTLNSKISYRIVSQEPANTPVFYLNKDTGEIYTTSFALDREEHSSYSLTVEARDGNGQITDKPVQQAQVQIRILDVNDNIPVVEYGTYEGVVEENQVNVEVMRIKVTDADEVGSDNWLANFTFASGNEGGYFHIETDTQTNEGIVTLVKELDYEEMKKLDLSILVTNKAAFHKSVVSKYKATPIPITVKVKNVVEGIHFKSSTVTFRASEAMDRSSLSRSIGKFQVFDEDTGKAADVTYVKVQDTDNWVSVDSETSEIKLVKIPDFESRHVQNGTYTVKVVAISKGHPQKTVTGTILINVEDVNDNCPVLVDSVRSVCADAPYVNVTAEDLDGPRNSGPFTFSIIDQPPGTAQKWKITHQESTSVLLQPSERKLGRSEIPFLISDSQGFSCLERQVLQLTVCECLEGGGCVAALYDNYVGLGPAAIALMILALLLLLLVPLLLLMCHCGEGAKGFAPIPGTIEMLHPWNNEGAPPEDKVVPSLLVTDHVENSGVRGGGGGGGGGVGGVKLKEGVSSSASITKGQHELIEVDGRWEEHRSLLTSGLTHHVATAGSIAANDTTIRTTRATGASRDMSGARGAVAMNEELLRSYFTEKAASYAGEDDFHLAKDCLLVYSQEDTDSLRGSVGCCSFIEGELDDLFLDDLGPKFKTLAEVCLGRKIDMDVDIEQRQKPVRETSVNAAPGSHYTQVTASSESTYSSNSGFQAPKPLHGAHTEKVTQEVVTESSVSSRQSQKVVPPPNPVASGNIIVTETSYAAGSAVPPSTVILAPRQPQSLIVTERVYAPASTLVDQHYANEENVLVTERVIQPNGGIPKPLEVTQHLNDAQYVMVRERESILAPSSGVQPTLAMPSVAAGGQNVTVTERVLTPASTLQSSYQIPSEASIKARKTVLSSVGGMGPLPNLDLEESGPPNSTVTTSSTRVTKHSTVQHSYS

SEQ ID NO: 16 - protein, 1115 aa, Macaca mulatta
MARSPGRAYALLLLLICFNVGSGLYLQVSSTRNENKLLPKHPHLVRQKRAWITAPVALREGEDLSKKNPIAKIHSDLTEERGLKVTYKYTGKGITEPPFGIFVFNKDTGELNITSILDREETPFFLLTGYALDERGNNVEKPLELRIKVLDINDNEPVFTQDVFVGSVEELNTLVMKINATDADEPNTLNSKISYRIVSLEPAYPPVFYLNKDTGEIYTTSVTLDREEHSSYTLTVEARDGNGEVTDKPVKQAQVQIRILDVNDNIPVVENKVLEGMVEENQVNVEVTRIKVFDADEIGSDNWLANFTFASGNEGGYFHIETDAQTNEGIVTLIKEVDYEEMKNLDFSVIVTNKAAFHKSIRSKYKPTPIPIKVKVKNVKEGIHFKSSVISTYVSESMDKSSKGQIIGKFQAFDEDTGLLARARYVKLEDRDNWISLDSVTSEIKLAKLPDFESRYVQNGTYTVKIVAISEDYPRKTITGTVLIYVEDINDNCPTLIEPVQTICNDAQYVNVTAEDLDGHPNSGPFSFSVIDKPPGMAEKWKIVHQESTSVLLQQSEKKLGRSEIQFLISDNQGFSCPEKQMLTLTVCECLHGSGCREAQRDSYVGLGPAAIALMILAFLLLLLVPLLLLMCHCGKGGKGFTPIPGTIEMLHPWNNEGAPPEDKVVPSFLPVDQGESLGGRNGVGGMAKEATMKGSSSASFAKGQHEMSEMDGRWEEYRSLLSGRATQVTGATGAIMTTETTKTTRATGASRDMAGAQAAAVALNEEFLRNYFSDKAASYTEEDENHTAKDCLLVYSQEETESLNASIGCCSFIEGELDDRFLDDLGLKFKTLAEVCLGQKIDINMEIEQRQKPATETSMNTASRSAYEQTMVNSENTYSSGSSFPVPKSLQEANAEKVTQEIVTERSVSSRQAQKVATPLPDPVASRNIIATETSYVTGSTMPPSTVILGPRQPQSLIVTERVYAPASALVDQPYANEGTVVVTERVIQPHGGGSNPLEGTQHLQDAPYVMVRERESFLAPSSGVQPTLAMPNIAVGQNVTVTERVLAPASTLQSSYQIPTENSVKARNTMVSGAGVPGPLPDFGLEESGHSNSTITTSSTRVTKHSTVQHSYS

SEQ ID NO: 17 - protein, 1119 aa, Canis lupus
MARSAEGAALLVLICFYFGNGLHLEVLNARNGNTLLPKHRHLVRQKRAWITAPVALREGEDLSKRNPIAKIHSDVAEERGLKITYKYTGKGITEPPFGVFVFNKDTGELNVTKILDREETPSFLLIGYALDEKGNNLEKPLELRIKVLDINDNEPVFTQDVFAGSIEELSAADTLVMKISATDADEPNTLNSKISYRIVSQEPTYPPVFYLNKDTGEIYTTSFTLDREEHSSYTLTVEARDGNGQITDKPVKQAQVQIRILDVNDNIPVVKNEMNEGVVEENQANVEVMRIKVFDADEIGSDNWLANFTFVSGNEMGYFHIETDTQTNEGIVTLIKEVDYEEVKNLDFSIVVTNKAAFHKSVKNKYKPTSIPFKIKVKNVKEGIHFKSSTVSVHVSESMDKSSQSQIIGKFQVFDEDTGQQARVKYAKLEDIDNWISVDSVTSEIRLVKIPDFESRYVQNGTYTAKILAISESYPRKTITGTVVITVEDINDNCPTLVDPVQNICDDEQYVNVTAEDLDGPQNSGPFSFSIIDKPAGMAEKWQIVHQESTSVLLQQKEQKLGRSEIQFLISDSQGFSCSEKQVLQLTVCKCLDGSGCVEPLRGTYVGLGPAAIALIIFALLLLLLVPLLLLMCHCGEGAKGFTPIPGTIEMLHPWNNEGAPPEDKVVPLHLTADHGESVAADIRLGVVTTKETTKESSSALIKDHHEMSEIDGRWEEHRSLISGGVTQVTGTTGANIGAEALRITKATGSSRDLAGAQAAAAAVNEEFLKSYFTEKAASYTDEDDIHTGKDCLLVYSQEDTASLHGSIGCCSFIEGELDDGFLDDLGFKFRTLAEICLGQKIDMDVEMEQRQKHAGESSMKAASHSFSEQTNINSENAYFSGSSFQVPKPLDEANAEKVTQEIVTESSVSSRQGHKVATLLPDPLASGNVVVTETSYGTGSTMPPSTVVLGPGQSQALIVTERVYAPAPPLVEQRYANDTNVMVTETIIQPNGGFPGPLEGTQHLPDGRYVVVRERESFLAPRSGAQPTLAMPGVAVGQNMTVTERVQTPASSYWIPAQTSVMARKTEVSGAGVRGPLPNCSLEESGHSNSMLTGHSNSTLTASSTRVSKHSIVQHSYS

SEQ ID NO: 18 - protein, 1142 aa, Danio rerio
MARRISPVVAFLLCFGLSHFFEAEARLQHSVALHRQKREWIVPPQILEENVDYTKKDFIAKIRSDKEVAHMKYLRYSLRGVGADQEPFNLFVVNPETGYVRITGILDRESISQYNLSGIALFTDGSIAENDIGLRIKVKDQNDNAPVFGVMNPGAVDELSAVGTEVMRLNCFDADEPGNPNSQIKYEIVDQQPAGQSMFTVENNRRVVVANPNLDRETVDQYVLLVKASDLNGAPGGNAATGTVTIKINDVNDNVPTLGGPYEASIEENTEKVEVMRLKVSDLHLKGTDNWEGDCYIASGNEAGYFSIHMDPKTNEAVLMLDKAVDYEDVKDLNLGIGVANKAPFHPSVSGGSQGATISFGGSGGGAGSGAAGGAGAMGGASGSGGGTGASSWSSSGVPLYNVNIKVKNQPEGPKFFPGTKAIPISEGKAFDSTEIIARYPAIDTDTGKEATNVKYIKSSDPDNWLTIDEKTGAIRMNKAPDRESKYLVNGTYYAKVLSITQDLPSKTATGTIAIQVEDFNDHCPTLISNVQTLCTDKDAVLVTAKDEDAPPNGAPFDFNIVSEGTAGKWTVEYMNDTTAIFRTHEKLWPGPHELMVEVTDQQGLKCPEPQKLQVDVCTCKNQGGCDRTATGDAKKGSRLGPAGIGLLLLALLALLLIPLLLLLCTCGMTGAFTDMPFETKVHLISSNTEGQGEDRDVPLMCPPSNVDGMGFMTKDYMAVGAMHSAGLGLGVGAGVGAAGFLESTSTMGGRGYNEMELDYMNSIGRNNAYSSRDMAGDFDGMALSDGYLCEYYSQKSRVVDGFGKDDPMVYDYEGKGSPVGSVGCCSLLEDQNDLEFLNDLGPKFTTLADICGGKKTEIPAPAPAPLPPPPKPVVDRSEVVSSTTNILNTGNIATNRVNTVNVASNMATASSTRVENVLVTDNRPTMITSVHPAPTLLVQPQPMYYMVEQQPSTVLVAERPAMTQGMYVLNSGPVAEGMVVQGGNIAANTLTRGERMVLVFRGGPAQALNNGMLHTSNLSGSQLLLVDGGATSGQVLQGTIQRGVAGSQGLMFVDGQGGQVIQGSINNGISTHGGSQNVFYVENKGGSSVVQGGLQMGKASTAGSLIGDVGIGGSSVKITQNPSSHKVVVQERKVVTTQSVK

SEQ ID NO: 19 - protein, 470 aa, Canis lupus
MESVLCWVFLVSILKGVQGEVQLVESGGDLVKPGGSLRLSCVASGFTFSDYGMSWVRQSPGKGLQWVAAVSNRGDTYYADAVKGRFTISRDNAKNTLYLQMSSLKAEDTAIYHCVTGVWPRHYYGMDHWGNGTSLFVSSASTTAPSVFPLAPSCGSTSGSTVALACLVSGYFPEPVTVSWNSGSLTSGVHTFPSVLQSSGLYSLSSTVTVPSSRWPSETFTCNVVHPASNTKVDKPVPKESTCKCISPCPVPESLGGPSVFIFPPKPKDILRITRTPEITCVVLDLGREDPEVQISWFVDGKEVHTAKTQPREQQFNSTYRVVSVLPIEHQDWLTGKEFKCRVNHIGLPSPIERTISKARGQAHQPSVYVLPPSPKELSSSDTVTLTCLIKDFFPPEIDVEWQSNGQPEPESKYHTTAPQLDEDGSYFLYSKLSVDKSRWQQGDTFTCAVMHEALQNHYTDLSLSHSPGK

SEQ ID NO: 20 - protein, 331 aa, Canis lupus
ASTTAPSVFPLAPSCGSTSGSTVALACLVSGYFPEPVTVSWNSGSLTSGVHTFPSVLQSSGLYSLSSTVTVPSSRWPSETFTCNVVHPASNTKVDKPVPKESTCKCISPCPVPESLGGPSVFIFPPKPKDILRITRTPEITCVVLDLGREDPEVQISWFVDGKEVHTAKTQPREQQFNSTYRVVSVLPIEHQDWLTGKEFKCRVNHIGLPSPIERTISKARGQAHQPSVYVLPPSPKELSSSDTVTLTCLIKDFFPPEIDVEWQSNGQPEPESKYHTTAPQLDEDGSYFLYSKLSVDKSRWQQGDTFTCAVMHEALQNHYTDLSLSHSPGK

SEQ ID NO: 21 - protein, 468 aa, Canis lupus
MESVFCWVFLVVILKGVQGEVQLVESGGDLVKPGGSLRLSCVASGFTFSSYYMHWIRQAPGKGLQRVAHIRGDGRTTHYADAMKGRFTISRDNAKNTLYLQMNSLTVEDTAIYYCVKDIYYGVGDYWGQGTLVTVSSASTTAPSVFPLAPSCGSTSGSTVALACLVSGYFPEPVTVSWNSGSLTSGVHTFPSVLQSSGLHSLSSMVTVPSSRWPSETFTCNVVHPASNTKVDKPVFNECRCTDTPPCPVPEPLGGPSVLIFPPKPKDILRITRTPEVTCVVLDLGREDPEVQISWFVDGKEVHTAKTQSREQQFNGTYRVVSVLPIEHQDWLTGKEFKCRVNHIDLPSPIERTISKARGRAHKPSVYVLPPSPKELSSSDTVSITCLIKDFYPPDIDVEWQSNGQQEPERKHRMTPPQLDEDGSYFLYSKLSVDKSRWQQGDPFTCAVMHETLQNHYTDLSLSHSPGK

SEQ ID NO: 22 - protein, 331 aa, Canis lupus
ASTTAPSVFPLAPSCGSTSGSTVALACLVSGYFPEPVTVSWNSGSLTSGVHTFPSVLQSSGLHSLSSMVTVPSSRWPSETFTCNVVHPASNTKVDKPVFNECRCTDTPPCPVPEPLGGPSVLIFPPKPKDILRITRTPEVTCVVLDLGREDPEVQISWFVDGKEVHTAKTQSREQQFNGTYRVVSVLPIEHQDWLTGKEFKCRVNHIDLPSPIERTISKARGRAHKPSVYVLPPSPKELSSSDTVSITCLIKDFYPPDIDVEWQSNGQQEPERKHRMTPPQLDEDGSYFLYSKLSVDKSRWQQGDPFTCAVMHETLQNHYTDLSLSHSPGK

SEQ ID NO: 23 - protein, 473 aa, Canis lupus
MESVLFWVFLVTILKGVQGEVRLVESGGTLVKPGGSLKLSCVASGFTFRRYSMDWVRQAPGKSLQWVAGINGDGTGTSYSQTVKGRFTISRDNAKNTLYLQINSLRAEDSAVYYCAKSWSRNGDLDYWGQGTLVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 24 - protein, 335 aa, Canis lupus
ASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 25 - protein, 474 aa, Canis lupus
MESVLYWVFLVAILKGVQGDVQLVESGGDLVKPGGSLRLSCVASGFTFSSCAMSWVRQSPGKGPQWVATIRYDGSDIYYADAVKGRFSISRDNAKNTVYLQMNSLRAEDTAVYYCAKAPPYDSYHYGMDYWGPGTSLFVSSASTTAPSVFPLAPSCGSQSGSTVALACLVSGYIPEPVTVSWNSVSLTSGVHTFPSVLQSSGLYSLSSMVTVPSSRWPSETFTCNVAHPATNTKVDKPVAKECECKCNCNNCPCPGCGLLGGPSVFIFPPKPKDILVTARTPTVTCVVVDLDPENPEVQISWFVDSKQVQTANTQPREEQSNGTYRVVSVLPIGHQDWLSGKQFKCKVNNKALPSPIEEIISKTPGQAHQPNVYVLPPSRDEMSKNTVTLTCLVKDFFPPEIDVEWQSNGQQEPESKYRMTPPQLDEDGSYFLYSKLSVDKSRWQRGDTFICAVMHEALHNHYTQISLSHSPGK

SEQ ID NO: 26 - protein, 333 aa, Canis lupus
ASTTAPSVFPLAPSCGSQSGSTVALACLVSGYIPEPVTVSWNSVSLTSGVHTFPSVLQSSGLYSLSSMVTVPSSRWPSETFTCNVAHPATNTKVDKPVAKECECKCNCNNCPCPGCGLLGGPSVFIFPPKPKDILVTARTPTVTCVVVDLDPENPEVQISWFVDSKQVQTANTQPREEQSNGTYRVVSVLPIGHQDWLSGKQFKCKVNNKALPSPIEEIISKTPGQAHQPNVYVLPPSRDEMSKNTVTLTCLVKDFFPPEIDVEWQSNGQQEPESKYRMTPPQLDEDGSYFLYSKLSVDKSRWQRGDTFICAVMHEALHNHYTQISLSHSPGK

SEQ ID NO: 27 - protein, 5 aa, synthetic construct
GGGGS

SEQ ID NO: 28 - protein, 6 aa, synthetic construct
IEGRMD

SEQ ID NO: 29 - protein, 20 aa, Mus musculus
METDTLLLWVLLLWVPGSTG

SEQ ID NO: 30 - protein, 1117 aa, Homo sapiens
MARTRDRVRLLLLLICFNVGSGLHLQVLSTRNENKLLPKHPHLVRQKRAWITAPVALREGEDLSKKNPIAKIHSDLAEERGLKITYKYTGKGITEPPFGIFVFNKDTGELNVTSILDREETPFFLLTGYALDARGNNVEKPLELRIKVLDINDNEPVFTQDVFVGSVEELSAAHTLVMKINATDADEPNTLNSKISYRIVSLEPAYPPVFYLNKDTGEIYTTSVTLDREEHSSYTLTVEARDGNGEVTDKPVKQAQVQIRILDVNDNIPVVENKVLEGMVEENQVNVEVTRIKVFDADEIGSDNWLANFTFASGNEGGYFHIETDAQTNEGIVTLIKEVDYEEMKNLDFSVIVANKAAFHKSIRSKYKPTPIPIKVKVKNVKEGIHFKSSVISIYVSESMDRSSKGQIIGNFQAFDEDTGLPAHARYVKLEDRDNWISVDSVTSEIKLAKLPDFESRYVQNGTYTVKIVAISEDYPRKTITGTVLINVEDINDNCPTLIEPVQTICHDAEYVNVTAEDLDGHPNSGPFSFSVIDKPPGMAEKWKIARQESTSVLLQQSEKKLGRSEIQFLISDNQGFSCPEKQVLTLTVCEVLHGSGCREAQHDSYVGLGPAAIALMILAFLLLLLVPLLLLMCHCGKGAKAFTPIPGTIEMLHPWNNEGAPPEDKVVPSFLPVDQGGSLVGRNGVGGMAKEATMKGSSSASIVKGQHEMSEMDGRWEEHRSLLSGRATQFTGATGAIMTTETTKTARATGASRDMAGAQAAAVALNEEFLRNYFTDKAASYTEEDENHTAKDCLLVYSQEETESLNASIGCCSFIEGELDDRFLDDLGLKFKTLAEVCLGQKIDINKEIEQRQKPATETSMNTASHSLCEQTMVNSENTYSSGSSFPVPKSLQEANAEKVTQEIVTERSVSSRQAQKVATPLPDPMASRNVIATETSYVTGSTMPPTTVILGPSQPQSLIVTERVYAPASTLVDQPYANEGTVVVTERVIQPHGGGSNPLEGTQHLQDVPYVMVRERESFLAPSSGVQPTLAMPNIAVGQNVTVTERVLAPASTLQSSYQIPTENSMTARNTTVSGAGVPGPLPDFGLEESGHSNSTITTSSTRVTKHSTVQHSYS

SEQ ID NO: 31 - protein, 227 aa, Homo sapiens
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 32 - protein, 229 aa, Homo sapiens
ESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLGSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

(19) United States
(12) Patent Application Publication
(10) Pub. No.: US 2024/0279304 A1
(43) Pub. Date: Aug. 22, 2024

(54) DSG2 COMPOSITIONS AND METHODS

(71) Applicant: Arvada Therapeutics, Inc., Brookline, MA (US)

(72) Inventors: Shi Yin Foo, Brookline, MA (US); Ryan Edward Tyler, Arlington, MA (US)

(73) Assignee: Arvada Therapeutics, Inc., Brookline, MA (US)

(21) Appl. No.: 18/652,115

(22) Filed: May 1, 2024

Related U.S. Application Data
(63) Continuation of application No. PCT/US2022/079106, filed on Nov. 2, 2022.
(60) Provisional application No. 63/274,707, filed on Nov. 2, 2021.

(51) Int. Cl. C07K 14/705; A61K 38/00; A61K 38/17; A61K 39/395; A61P 9/06

(52) U.S. Cl. CPC C07K 14/705; A61K 38/177; A61K 39/39516; A61K 39/3955; A61P 9/06; A61K 38/00; C07K 2319/30

(57) ABSTRACT

The disclosure generally relates to compositions and methods of treating a disease by administering compositions disclosed herein. Provided herein are DSG2 fusion polypeptides and related methods including methods of treatment and improving cardiomyocyte function.

### CROSS REFERENCE TO RELATED APPLICATIONS

This application, filed under 35 U.S.C § 111(a), is a continuation of PCT Application No. PCT/US2022/079106, filed on Nov. 2, 2022, which claims the benefit of priority to U.S. Provisional Patent Application No. 63/274,707, filed on Nov. 2, 2021. The above-referenced patent applications are herein incorporated by reference in their entireties.

### SEQUENCE LISTING

The present application is being filed along with a Sequence Listing in XML format according to WIPO Standard ST26. The Sequence Listing file, entitled 10383-108749-06.xml, was created on Apr. 19, 2024, and is 48,278 bytes in size. The information in electronic format of the Sequence Listing is incorporated herein by reference in its entirety.

### BACKGROUND

Among the modulators of disease progression, a dysregulation in the immune system is believed to play a central role. The immune system consists of a multicellular highly regulated and complex defense system that is characterized by a high interindividual variability in its response to injury and antigens. In its physiological condition it is programmed to discriminate between self- and foreign constituents, thereby interacting with and eliminating any structures that are recognized as foreign. This process can convert into a pathological situation in which self-tissue is attacked, resulting in autoimmune disease.

Circulating autoantibodies have been critically linked to heart disease. Their prevalence, mode of action, and potential therapeutic modulation are intensively investigated. Although a triggering injury to myocardium is believed to be the crucial initiating event, the genetic predisposition, environmental and epigenetic modulators, and other still unknown mechanisms are critical for development of the pathological antibody titers observed in peripheral blood and the intensity of inflammation in myocardial structures. In a prospective study, Caforio et al showed that circulating anti-heart autoantibodies may precede disease manifestation and are independent predictors of disease development (Caforio et al. Circulation. 2007; 115:76-83; the contents of which are herein incorporated by reference in its entirety).

Currently, therapeutic strategies for treating and/or managing autoantibodies associated with diseases, in particular heart diseases are lacking. The present disclosure provides DSG2 fusion polypeptide-based compositions and methods for treating diseases such as, but not limited to cardiac diseases, and infectious diseases.

### SUMMARY

The present disclosure provides compositions comprising isolated polypeptides. The polypeptides of the disclosure may include a whole or a portion of the DSG2 protein. In some embodiments, the isolated polypeptide is a desmoglein-2 (DSG2) fusion polypeptide. The DSG2 fusion polypeptide may include (a) a whole or a portion of a DSG2 protein; and/or (b) a whole or a portion of an immunoglobulin protein. The DSG2 protein may be a Homo sapiens DSG2 protein, a Mus musculus DSG2 protein, a Rattus norvegicus DSG2 protein, a Macaca mulatta DSG2 protein, a Canis lupus familiaris DSG2 protein, or a Danio rerio DSG2 protein. The immunoglobulin protein may be a human or a canine immunoglobulin protein.

In one embodiment, the DSG2 protein may be a Homo sapiens DSG2 protein (SEQ ID NO. 1). In one embodiment, the DSG2 protein may be a Mus musculus DSG2 protein (SEQ ID NO. 14). In one embodiment, the DSG2 protein may be a Rattus norvegicus DSG2 protein (SEQ ID NO. 15). In one embodiment, the DSG2 protein may be a Macaca mulatta DSG2 protein (SEQ ID NO. 16). In one embodiment, the DSG2 protein may be a Canis lupus familiaris DSG2 protein (SEQ ID NO. 17). In one embodiment, the DSG2 protein may be a Danio rerio DSG2 protein (SEQ ID NO. 18).

In one embodiment, the DSG2 polypeptide may include a portion of DSG2 protein. The portion of the DSG2 protein may include the extracellular region of DSG2 protein. In some aspects, the entire extracellular region of DSG2 may be included in the fusion polypeptide. In one embodiment, the entire extracellular region of DSG2 includes the amino acid sequence of SEQ ID NO: 3. Embodiments of the disclosure may also include a portion of the extracellular region of DSG2. For example, a portion of the extracellular region may be extracellular cadherin domain 1 (EC1), extracellular cadherin domain 2 (EC2), extracellular cadherin domain 3 (EC3), extracellular cadherin domain 4 (EC4), and/or extracellular anchor domain (EA). In some aspects, the DSG2 fusion polypeptides include 2 domains of the extracellular region. For example, the two domains may be EC4EA, EC1EC2, EC2EC3, EC3EC4, EC1EA, EC1EC3, EC2EC4, and/or EC3EA. In some aspects, the DSG2 fusion polypeptides include three domains of the extracellular region. For example, the three domains may be EC1EC3EA, EC1EC4EA, EC1EC3EA, EC3EC4EA, EC1EC2EC3, EC2EC3EC4, and/or EC2EC4EA. In some aspects, the DSG2 fusion polypeptides may include four domains of the extracellular region. For example, the three domains may be EC1EC2EC4EA, EC2EC3EC4EA, EC1EC2EC3EC4EA, EC1EC2EC3EC4, and/or EC1EC2EC3EA.

DSG2 fusion polypeptides may include a portion of an immunoglobulin. The portion may be an Fc region, an Fab region, a heavy chain variable (VH) domain, a heavy chain constant domain, a light chain variable (VL) domain, and/or a light chain constant domain. In one aspect, the portion of the immunoglobulin may be an Fc region. The immunoglobulin may be an IgG, an IgM, an IgA, an IgD and/or an IgE. As a non-limiting example, the immunoglobulin may be IgG. The compositions may include an IgG such as IgG1, IgG2, IgG3, and/or IgG4. The Fc region may be a human Fc region or a canine Fc region. Non-limiting examples of portions of immunoglobulin useful in the present disclosure include a human IgG1 Fc region (SEQ ID NO: 5), a human IgG2 Fc region (SEQ ID NO: 7), a human IgG3 Fc region (SEQ ID NO: 9), or a human IgG4 Fc region (SEQ ID NO: 11), a canine IgG heavy chain D Fc region (SEQ ID NO: 20), a canine IgG heavy chain A Fc region (SEQ ID NO: 22), a canine IgG heavy chain B Fc region (SEQ ID NO: 24), or a canine IgG heavy chain C Fc region (SEQ ID NO: 26), a human IgG1 heavy chain constant domain (SEQ ID NO: 4), a human IgG2 heavy chain constant domain (SEQ ID NO: 6), a human IgG3 heavy chain constant domain (SEQ ID NO: 8), or a human IgG4 heavy chain constant domain (SEQ ID NO: 10), a canine IgG heavy chain constant domain chain D (SEQ ID NO: 19), a canine IgG heavy chain constant domain chain A (SEQ ID NO: 21), a canine IgG heavy chain constant domain chain B (SEQ ID NO: 23), or a canine IgG heavy chain constant domain chain C (SEQ ID NO: 25).

Polypeptides of the disclosure may further include a linker sequence. The linker may be from about 5 amino acids to about 50 amino acids in length. In one embodiment, the linker may be GGGGS (SEQ ID NO: 12). In another aspect, the linker may be EAAAK (SEQ ID NO: 13), GGGGS (SEQ ID NO: 27) or IEGRMD (SEQ ID NO: 28).

One embodiment is a pharmaceutical composition for use in treatment of a disease or disorder caused by anti-desmoglein-2 (DSG2) autoantibodies. The composition comprises a fusion polypeptide which blocks binding of anti-DSG2 antibodies to the extracellular domain of DSG2. The fusion polypeptide comprises an extracellular region of a human DSG2 protein having at least about 85% sequence identity with amino acid residues 50 to 609 of SEQ ID NO: 1, or a portion thereof, and an affinity tag. The portion of the extracellular region of the human DSG2 protein may include any one of or a combination of DSG2 domains selected from the group consisting of: extracellular cadherin domain 1 (EC1), extracellular cadherin domain 2 (EC2), extracellular cadherin domain 3 (EC3), extracellular cadherin domain 4 (EC4), and extracellular anchor domain (EA). The affinity tag may be an Fc region of an immunoglobulin such as IgG1 or a variant thereof. The variant may have the sequence of SEQ ID NO: 31. The affinity tag may be an Fc region of IgG4 or a variant thereof. The variant may have the sequence of SEQ ID NO: 32. Alternatively, the affinity tag may be provided by a smaller protein or peptide such as a polyhistidine peptide. Some embodiments of the fusion polypeptides include a linker sequence located between the extracellular region of a human DSG2 protein or portion thereof, and the affinity tag. In some embodiments, the linker sequence is SEQ ID NO: 12, 13, 27 or 28. In some embodiments, the disease or disorder is an arrhythmia such as arrhythmogenic right ventricular cardiomyopathy (ARVC), sarcoidosis, or dilated cardiomyopathy. In some embodiments, the disease or disorder is a cardiomyopathy such as arrhythmogenic right ventricular cardiomyopathy (ARVC), sarcoidosis, or dilated cardiomyopathy. The arrhythmia or cardiomyopathy may be caused by a virus such as SARS-COV2, adenovirus, hepatitis virus, hepatitis C virus, parvovirus, herpes simplex virus, echovirus, Epstein-Barr virus, rubella, cytomegalovirus, or HIV.

The present disclosure provides methods of reducing proarrhythmic phenotypes in a cardiomyocyte. Such methods may involve contacting the cardiomyocyte with the compositions of the disclosure. In some embodiments, the proarrhythmic phenotype may be associated or caused by anti-DSG2 antibodies. The present disclosure also provides methods of reducing or correcting the proarrhythmic phenotype by contacting the cardiomyocyte with the compositions of the disclosure. Reduction or correction of the proarrhythmic phenotype may be measured using cardiomyocyte sodium spike (μV/m). The present disclosure also provides methods of treatment using the compositions described herein.

The present disclosure also provides a method of treating a condition associated with scrum DSG2 autoantibodies. Such methods may include administering the compositions described herein or cells expressing the compositions described herein to a subject. In some embodiments, the condition may be an arrhythmia. In some embodiments, the condition may be a cardiomyopathy. In some aspects, the condition may be an autoimmune disorder.

The present disclosure provides methods of treating arrhythmia and/or cardiomyopathy in a subject. Such methods may include contacting the subject with the isolated polypeptides or the cells of the disclosure followed by measuring one or more symptoms or clinical findings associated with arrhythmia such abnormal beats on an electrocardiogram, dizziness, lightheadedness, palpitations, chest pain, shortness of breath, reduced ejection fraction, poor cardiac output and/or heart failure. Such methods may include contacting the subject with the isolated polypeptides or the cells of the disclosure followed by measuring one or more symptoms associated with cardiomyopathy such as arrhythmia, palpitations, myocarditis, heart failure, poor cardiac output, and/or reduced ejection fraction. As a non-limiting example, the cardiomyopathy may be arrhythmogenic right ventricular cardiomyopathy (ARVC), sarcoidosis, dilated cardiomyopathy. The cardiomyopathy may also be caused by a virus (e.g., SARS-COV2, adenovirus, hepatitis virus, hepatitis C virus, parvovirus, herpes simplex virus, echovirus, Epstein-Barr virus, rubella, cytomegalovirus, or HIV), a bacterium (Staphylococcus, Streptococcus, or Borrelia), a parasite (Trypanosoma or Toxoplasma) or a fungus (Candida, Aspergillus, or Histoplasma). In some embodiments, the subject may have detectable levels of anti-DSG2 antibodies in their serum.

The present disclosure also provides method of treating a cardiac abnormality in a subject using the DSG2 fusion polypeptides described herein. In some embodiments, the subject may have scrum anti-DSG2 antibodies. The present disclosure also provides a method of a method of reducing anti-DSG2 antibodies in a subject using the DSG2 fusion polypeptides described herein. In some embodiments, the anti-DSG2 antibody levels may be reduced by about 1%, 5%, 10%, 15%, 20%, 25%, 30%, 35%, 40%, 45%, 50%, 55%, 60%, 65%, 70%, 75%, 80%, 85%, 90%, 95%, or 100%.

Provided herein are compositions comprising the DSG2 fusion polypeptides. Also provided here in are compositions comprising at least one therapeutic agent. In some embodiments, the compositions of the disclosure may comprise a combination of DSG2 fusion polypeptides and at least one therapeutic agent described herein. In some embodiments, the compositions include therapeutic agents only. Non-limiting examples of therapeutic agents include anti-CD20 antibody, FcRn-blocking antibody, intravenous immunoglobulins (IVIG), and/or complement inhibitors such as eculizumab. Also provided herein is a method of treating a condition associated with serum anti DSG2 autoantibodies using the DSG2 fusion polypeptides and/or the therapeutic agents described herein. In some embodiments, the condition associated with serum anti-DSG2 auto antibodies may be a cardiac disorder, or an infectious disease. The cardiac disorder may be ARVC, sarcoidosis or dilated cardiomyopathy, or any cardiac disease associated with anti-DSG2 antibodies.

### DESCRIPTION OF THE FIGURES

The foregoing and other objects, features and advantages will be apparent from the following description of particular embodiments of the disclosure, as illustrated in the accompanying drawings. The drawings are not necessarily to scale; emphasis instead being placed upon illustrating the principles of various embodiments of the disclosure.

FIG. 1A shows the % inhibition of anti-DSG2 antibody and ARVC serum using DSG2FP #1. FIG. 1B shows the % inhibition of anti-DSG2 antibody and ARVC serum using DSG2FP #2. FIG. 1C shows the % inhibition of anti-DSG2 antibody and ARVC serum using DSG2FP #3. FIG. 1D shows the % inhibition of anti-DSG2 antibody and ARVC serum using DSG2FP #4. The DSG2 fusion polypeptides were subject to size exclusion chromatography and different fractions were examined. Fraction “C” had a predicted MW of ˜350 kDa and was used in additional experiments. In FIG. 1B, FIG. 1C, FIG. 1D, whole protein preparations (“Whole preparation”) were compared to “Fraction C.”

FIG. 2A is a first series of voltage (μV) vs time (msec) graphs showing that lidocaine and anti-DSG2 antibodies dampen the sodium spike in human iPSC cardiomyocytes in multiple-electrode array (MEA) assay. The dampening of the sodium spike was reversed by the addition of the DSG2FP #1 (labelled as in FIG. 2A as “DSG2 fusion polypeptide”) showing that DSG2FP #1 completely blocks the effect of the anti-DSG2 antibodies on the sodium spike.

FIG. 2B is a second series of voltage (μV) vs time (msec) graphs showing that control tests of vehicle, rabbit IgG and anti-VCAM-1 antibodies have no effect on the sodium spike in the MEA assay.

FIG. 3 shows cell index upon treatment with anti-DSG2 antibodies and anti-DSG2 antibodies plus DSG2FP #1 (labelled in FIG. 3 as “decoy”) in human iPSC cardiomyocytes in multiple-electrode array (MEA) assay showing that DSG2FP #1 completely blocks the effect of the anti-DSG2 antibodies.

FIG. 4A is graph of the anti-DSG2 antibody signal in a detection assay.

FIG. 4B shows the correlated correlation of percentage inhibition with the signal to background ratio in the different groups. The control group and ARVC weak clinical data group data points are clustered on the y axis suggesting little no difference in the signal ratio across these two groups.

FIG. 5 provides a series of bar charts indicating blocking of ARVC patient sera binding to the DSG2 extracellular domain by a series of recombinant DSG2 fusion proteins assessed in an electrochemical immunoassay.

FIG. 6 is bar chart indicating blocking of anti-DSG2 antibodies binding to the DSG2 extracellular domain by recombinant DSG2(50-609)-IgG4 Fc fusion proteins with and without a linker, assessed in an electrochemical immunoassay.

### DETAILED DESCRIPTION

### I. Introduction

The production of autoantibodies against self-proteins, called autoantigens, is a characteristic of many autoimmune diseases. Autoantibody immunoreactivity in a patient's body fluids provides key diagnostic information when autoimmune disease is suspected. The spectrum of autoantibodies is often clinically informative for a given autoimmune disease. Some autoimmune diseases harbor autoantibodies against only one or a few target autoantigens, but in other conditions autoantibodies against multiple targets may co-exist. Among the seventy most common autoimmune diseases, approximately 100 out of the estimated 20,000 human proteins encoded by the genome are thought to comprise the most common antigenic targets. However, an increasing number of autoantibodies have been discovered in rare disorders suggesting additional diseases are likely to exhibit autoantibody-associated autoimmunity.

The treatment of many autoimmune diseases remains sub-optimal due to varying degrees of efficacy and the side-effects of available interventions. Advances in autoimmune disease therapeutics will require disease-specific information including how autoantibodies participate in pathogenesis.

Desmoglein-2 is one of several cardiac cadherin proteins. Cadherins are calcium-dependent adhering molecules providing mechanical attachment between cells in multiple tissues. Typically, three calcium ions (12 in total) are pocketed into binding motifs present between each pair of five successive extracellular cadherin (EC) domains, providing the crescent shape required to bring cadherin domains from opposing cells into a 90-degree conformation for trans binding. This 90-degree conformation is critical for binding as it allows for tryptophan residues on each EC1 domain to be inserted simultaneously into the hydrophobic pocket of the alternate cadherin. Interfering with this orientation, such as through missense mutations, or antibody binding to proximal extracellular DSG2 domains, may reduce binding and adhesion. Chatterjee et al. identified autoantibodies to the cardiac desmoglein-2 (DSG2) protein as a common feature in the sera of patients with ARVC (Chatterjee D, et al., Eur. Heart J. 2018; 39(44): 3932-3944; the contents of which are herein incorporated by reference in its entirety). These autoantibodies were specific to ARVC, as they were essentially absent in two independent sets of control sera, as well as sera from subjects with other forms of heritable cardiomyopathy. Anti-DSG2 antibodies can also be found in some cases of sarcoidosis, a systemic inflammatory disease which results in granulomas in organs, such as, but not limited to the heart. Anti-DSG2 antibodies are also found in sarcoid patients with cardiac involvement (Suna et al. 2020, Eur. Heart Journal, Volume 41, Issue Supplement 2, November 2020, chaa946.2127; the contents of which are herein incorporated by reference in its entirety). Patients with a diagnosis of dilated cardiomyopathy may have mutations in the same desmosome proteins that are associated with ARVC. These observations suggest that some dilated cardiomyopathy patients may actually have ARVC-like disease, mediated by anti-DSG2 autoantibodies, but have been diagnosed as dilated cardiomyopathy because they do not fit the typical age and/or presentation associated with ARVC. Anti-DSG2 autoantibodies are considered to arise when there is a combination of cardiac cellular damage and an activated immune system for example, in the setting of an infection known to affect the myocardium directly. Strategies targeting anti-DSG2 antibodies (e.g., DSG2 autoantibodies) may therefore be beneficial in the treatment of symptoms and diseases that arise from the presence of anti-DSG2 antibodies, including but not limited to COVID-19, post-COVID-19 syndrome and/or cardiac diseases e.g. ARVC.

The present disclosure provides compositions and methods related to DSG2 fusion polypeptides for targeting anti-DSG2 antibodies. The DSG2 fusion polypeptides of the disclosure may therefore be a viable therapeutic strategy in the treatment of diseases associated with anti-DSG2 antibodies and/or diseases associated with an autoimmune response such as, but not limited to, COVID-19, post-COVID-19 cardiac syndrome, and ARVC. DSG2 fusion polypeptides may also be used in the treatment of other diseases associated with cardiac cellular damage, such as, but not limited to, arrhythmogenic cardiomyopathy (AC), sarcoidosis, dilated cardiomyopathy with anti-DSG2 autoantibodies and viral infections, including but not limited to those caused by coxsackie virus, adenovirus, echoviruses, parvovirus, rubella and/or cytomegalovirus.

### II. Compositions

In some embodiments, the present disclosure provides compositions that include DSG2 fusion polypeptides. Compositions described herein, may be capable of binding to or interacting with anti-DSG2 antibodies. In one embodiment, the compositions of the disclosure may modulate the activity of anti-DSG2 antibodies. In one embodiment, the compositions of the disclosure may inhibit the activity of the anti-DSG2 antibodies.

In some embodiments, the present disclosure includes a DSG2 protein. In some aspects the DSG2 protein may be the whole DSG2 protein or a portion of the DSG2 protein. In some embodiments, the DSG2 protein may be fused to any other protein or fragment of a protein.

DSG2 fusion polypeptides of the present disclosure may include a protein tag. Protein tags are protein or peptide sequences included in recombinant proteins which are provided for various purposes and are usually placed at the N-terminus or C-terminus. In the present embodiments, the protein tags function mainly as affinity tags but may provide other advantages such as enhanced expression, solubility, bioactivity, and pharmacokinetic properties. Affinity tags are appended to proteins so that they can be purified from their crude biological source using an affinity technique. Examples of affinity tags include chitin binding protein (CBP), maltose binding protein (MBP), streptavadin and glutathione-S-transferase (GST). A common polyhistidine known as 6×His, or hexahistidine, is a widely used protein tag which binds to matrices bearing immobilized metal ions.

Solubilization tags are used, especially for recombinant proteins expressed in species such as E. coli, to assist in the proper folding in proteins and keep them from aggregating in inclusion bodies. These tags include thioredoxin (TRX) and poly(NANP). Some affinity tags have a dual role as a solubilization agent, such as MBP and GST. The Fc region of immunoglobulins is also useful and acts as both an affinity tag and a dimerization and solubilization agent, as well as providing a means for detecting protein expression using commercial ELISA kits. Addition of an IgG-Fc tag can also increase protein expression yield and influence the in vivo pharmacokinetics of recombinant proteins.

In some of the example embodiments described hereinbelow, the protein tag of the fusion protein is a polyhistidine tag having six consecutive histidine residues, denoted as 6×His. In other examples, the protein tag is an Fc region of an IgG, including IgG1 (SEQ ID NO: 5) or IgG4 (SEQ ID NO: 11). These fusion polypeptide embodiments display activity with respect to binding to anti-DSG2 antibodies.

The DSG2 fusion polypeptides may include the whole DSG2 protein or a portion of a DSG2 protein and an affinity tag including a whole or a portion of an immunoglobulin protein. In some embodiments, the DSG2 fusion polypeptides may further include a linker peptide. In some embodiments, the whole or a portion of DSG2 protein may be fused to a protein that is not an immunoglobulin. The DSG2 protein may be fused to a protein or a fragment of a protein such as an affinity tag such as TRX, poly(NANP), MBP, GST or polyhistidine that is capable of improving the expression and purification of DSG2 protein in vitro or in vivo.

In some embodiments, the protein tag of the fusion polypeptide is a PAS polypeptide tag. PAS sequences are hydrophilic, uncharged biological polymers with biophysical properties very similar to poly-ethylene glycol (PEG), whose chemical conjugation to drugs is an established method for plasma half-life extension. In contrast, PAS polypeptides offer fusion to a therapeutic protein on the genetic level, permitting production of fully active proteins and obviating in vitro coupling or modification steps (Schlapschy et al., Protein Eng. Des. Sel., 2013, 26(8), 489-501, incorporated herein by reference in its entirety). The process of adding a PAS polypeptide to a fusion protein is known as “PASylation”. In some embodiments, polyethylene glycol (PEG) is used as a non-protein tag alternative to incorporation of a PAS polypeptide. The process of adding a PEG moiety is known as “PEGylation”. PASylation or PEGylation of certain embodiments of the fusion protein may provide the ability to purify the fusion protein by size exclusion chromatography instead of affinity chromatography.

DSG2 mutations within intercalated discs in heart cells have been implicated in cardiac diseases including arrhythmia, dilated cardiomyopathy, and particularly ARVC (arrhythmogenic right ventricular cardiomyopathy). Chatterjee et al. identified autoantibodies to the cardiac DSG2 protein as a common feature in the sera of patients with ARVC (Chatterjee D, et al., Eur Heart J. 2018; 39(44):3932-3944; the contents of which are herein incorporated by reference in its entirety). These autoantibodies were specific to ARVC, as they were essentially absent in two independent sets of control sera, as well as sera from subjects with other forms of heritable cardiomyopathy. The present disclosure provides DSG2 fusion polypeptides as a therapeutic strategy for targeting DSG2 autoantibodies. In some embodiments, DSG2 fusion polypeptides of the present disclosure may bind to DSG2 autoantibodies. In some embodiments, binding of the DSG2 fusion polypeptides of the disclosure to the DSG2 autoantibodies precludes the binding of the autoantibodies to the endogenous DSG2 in a subject. In this aspect of the disclosure, the DSG2 fusion polypeptides function as a decoy protein or a ligand trap.

Chatterjee et al. propose that the DSG2 protein may include epitopes, which are exposed or released into the extracellular space and/or circulation and as a result of cardiomyocyte damage or desmosome mutations. Unmasking of these epitopes may also occur from any cardiac damage (such as, but not limited to, infective myocarditis, and/or cardiac trauma). In some embodiments, the compositions of the disclosure may not include any mutations. Such released DSG2 proteins may link with an antigen-presenting cell to stimulate a T-cell response, generating the observed autoantibodies. Unmasking of cryptic epitopes by gene mutations could contribute to other forms of autoimmunity. In some embodiments, DSG2 fusion polypeptides of the disclosure may include epitopes containing one or more mutations in DSG2.

The DSG2 fusion polypeptide may be a soluble and/or recombinant polypeptide. The arrangement of components in the DSG2 fusion polypeptide may be optimized to achieve the suitable protein expression and/or the intended therapeutic effect. In some embodiments, the DSG2 fusion polypeptide may include formats described herein. The formats provided herein include components from N terminus to C terminus delineated by a “;” between the components. Non-limiting examples of the formats of the DSG2 fusion polypeptides include, (i) the whole or a portion of DSG2 protein; Fc region (ii) Fc region; the whole or a portion of the DSG2 protein (iii) the whole or a portion of DSG2 protein; linker; Fc region (iv) Fc region; linker; the whole or a portion of the DSG2 protein (v) affinity tag; the whole or a portion of the DSG2 protein (vi) the whole or a portion of DSG2 protein; affinity tag (vii) the whole or a portion of the DSG2 protein; linker; affinity tag (viii) affinity tag; linker; whole or a portion of the DSG2 protein.

### DSG2 Protein

In some embodiments, the DSG2 fusion polypeptides of the present disclosure may include the entire DSG2 protein. The desmosomal cadherin desmoglein-2 (DSG2) is a transmembrane cell adhesion protein that is expressed in epithelial and non-epithelial tissues, such as the heart and gastrointestinal tract. DSG2 is an integral part of the desmosome unit, which is a major structure supporting cell-to-cell attachments and structural integrity. DSG2 has been shown to regulate numerous cellular processes, including proliferation and apoptosis. In some embodiments, the DSG2 protein is a human DSG2 protein (UniProt ID: Q14126; ENSEMBL ID: ENSP00000261590.8) which consists of 1,118 amino acids, and includes the amino acid sequence of SEQ ID NO: 1. In one embodiment, the DSG2 protein may be encoded by nucleic acid sequence of SEQ ID NO: 2 (NCBI Reference Sequence: NM_001943.5.; ENSMBL ID: ENST00000261590.13).

In some embodiments, the DSG2 fusion polypeptide of the present disclosure may be a fully processed DSG2 protein comprising amino acids 50-1118 of SEQ ID NO: 1.

The DSG2 protein may also include one or more mutations with respect to the sequence of SEQ ID NO: 1. In some embodiments, DSG2 protein mutation may be a mutation associated with a disease state. In one embodiment, the disease state may be arrhythmogenic right ventricular dysplasia/cardiomyopathy. In some embodiments, DSG2 fusion polypeptides of the disclosure may include epitopes containing one or more mutations in DSG2. As a non-limiting example, DSG2 fusion polypeptides may include one or more mutations in the region of amino acids 485-531 and/or amino acids 586-610 of SEQ ID NO: 1.

DSG2 belongs to the cadherin superfamily of cell adhesion proteins, which commonly feature the three distinct regions: an extracellular region, a transmembrane domain and an intracellular signaling region. In some embodiments, the extracellular region of DSG2 may have the amino acid sequence of SEQ ID NO: 3, which is amino acids 50-609 of SEQ ID NO: 1. The extracellular region of cadherin family of proteins contain a varying number of repeats of calcium-binding motifs, known as cadherin motifs or EC domains. DSG2 contains four EC domains herein referred to as EC1, EC2, EC3 and EC4. DSG2 also includes an extracellular anchor (EA) domain that is proximal to the membrane. In some embodiments, the DSG2 fusion polypeptides of the disclosure may include the entire extracellular region of DSG2. In some aspects, the DSG2 fusion polypeptide may include at least one domain, such as, but not limited to, EC1, EC2, EC3, EC4, and/or EA. In some embodiments the EC1 domain may be amino acids 50-155 of SEQ ID NO: 1. In some embodiments the EC2 domain may be amino acids 151-268 of SEQ ID NO: 1. In some embodiments the EC3 domain may be amino acids 264-384 of SEQ ID NO: 1. In some embodiments the EC4 domain may be amino acids 382-495 of SEQ ID NO: 1. In some embodiments the EA domain may be amino acids 491-609 of SEQ ID NO: 1. Table 1 provides the amino acid sequence of the DSG2 protein as well as the amino acid sequence of the extracellular region of DSG2. In some embodiments, the DSG2 proteins of the present disclosure may have at least 50%, 55%, 60%, 65%, 70%, 75%, 80%, 85%, 90%, 95%, 99% or 100% identity to any of the sequences in Table 1 or fragments of the sequences in Table 1.

In some embodiments, the DSG2 protein is a non-human DSG2 protein. In some embodiments, the DSG2 protein is a Mus musculus (mouse) DSG2 protein. In one embodiment, the DSG2 protein may include 1,122 amino acids, and comprise the amino acid sequence of SEQ ID NO: 14. In some embodiments, the DSG2 protein is a Rattus norvegicus (rat) DSG2 protein. In one embodiment, the rat DSG2 protein may include 1,128 amino acids, and comprise the amino acid sequence of SEQ ID NO: 15. In some embodiments, the DSG2 protein is a non-human primate (NHP) DSG2 protein. In some aspects, the NHP DSG2 protein is a Macaca mulatta DSG2 protein which may include of 1,115 amino acids, and comprise the amino acid sequence of SEQ ID NO: 16. In some embodiments, the DSG2 protein is a Canis lupus familiaris (dog) DSG2 protein. In some embodiments, the dog DSG2 protein may include 1,119 amino acids, and comprise the amino acid sequence of SEQ ID NO: 17. In some embodiments, the DSG2 protein is a Danio rerio (zebra fish) DSG2 protein. In some embodiments, the zebra fish DSG2 protein may which include 1,142 amino acids, and comprise the amino acid sequence of SEQ ID NO: 18.

TABLE 1
Sequences of the DSG2 protein and DSG2 extracellular domain

| SEQ ID | | |
|---|---|---|
| NO: | Sequence | Description |
| 1 | MARSPGRAYALLLLLICFNVGSGLHLQVLSTRNENKLLP | Human DSG2 |
| | KHPHLVRQKRAWITAPVALREGEDLSKKNPIAKIHSDLA | protein |
| | EERGLKITYKYTGKGITEPPFGIFVFNKDTGELNVTSILDR | Q14126 |
| | EETPFFLLTGYALDARGNNVEKPLELRIKVLDINDNEPVF | |
| | TQDVFVGSVEELSAAHTLVMKINATDADEPNTLNSKISY | |
| | RIVSLEPAYPPVFYLNKDTGEIYTTSVTLDREEHSSYTLT | |
| | VEARDGNGEVTDKPVKQAQVQIRILDVNDNIPVVENKV | |
| | LEGMVEENQVNVEVTRIKVFDADEIGSDNWLANFTFAS | |
| | GNEGGYFHIETDAQTNEGIVTLIKEVDYEEMKNLDFSVI | |
| | VANKAAFHKSIRSKYKPTPIPIKVKVKNVKEGIHFKSSVIS | |
| | IYVSESMDRSSKGQIIGNFQAFDEDTGLPAHARYVKLED | |
| | RDNWISVDSVTSEIKLAKLPDFESRYVQNGTYTVKIVAIS | |
| | EDYPRKTITGTVLINVEDINDNCPTLIEPVQTICHDAEYV | |
| | NVTAEDLDGHPNSGPFSFSVIDKPPGMAEKWKIARQEST | |
| | SVLLQQSEKKLGRSEIQFLISDNQGFSCPEKQVLTLTVCE | |
| | CLHGSGCREAQHDSYVGLGPAAIALMILAFLLLLLVPLL | |
| | LLMCHCGKGAKGFTPIPGTIEMLHPWNNEGAPPEDKVV | |
| | PSFLPVDQGGSLVGRNGVGGMAKEATMKGSSSASIVKG | |
| | QHEMSEMDGRWEEHRSLLSGRATQFTGATGAIMTTETT | |
| | KTARATGASRDMAGAQAAAVALNEEFLRNYFTDKAAS | |
| | YTEEDENHTAKDCLLVYSQEETESLNASIGCCSFIEGELD | |
| | DRFLDDLGLKFKTLAEVCLGQKIDINKEIEQRQKPATETS | |
| | MNTASHSLCEQTMVNSENTYSSGSSFPVPKSLQEANAEK | |
| | VTQEIVTERSVSSRQAQKVATPLPDPMASRNVIATETSY | |
| | VTGSTMPPTTVILGPSQPQSLIVTERVYAPASTLVDQPYA | |
| | NEGTVVVTERVIQPHGGGSNPLEGTQHLQDVPYVMVRE | |
| | RESFLAPSSGVQPTLAMPNIAVGQNVTVTERVLAPASTL | |
| | QSSYQIPTENSMTARNTTVSGAGVPGPLPDFGLEESGHS | |
| | NSTITTSSTRVTKHSTVQHSYS | |
| 3 | AWITAPVALREGEDLSKKNPIAKIHSDLAEERGLKITYKY | Human DSG2 |
| | TGKGITEPPFGIFVFNKDTGELNVTSILDREETPFFLLTGY | extracellular |
| | ALDARGNNVEKPLELRIKVLDINDNEPVFTQDVFVGSVE | domain (amino |
| | ELSAAHTLVMKINATDADEPNTLNSKISYRIVSLEPAYPP | acids 50-609 of |
| | VFYLNKDTGEIYTTSVTLDREEHSSYTLTVEARDGNGEV | DSG2 protein) |
| | TDKPVKQAQVQIRILDVNDNIPVVENKVLEGMVEENQV | |
| | NVEVTRIKVFDADEIGSDNWLANFTFASGNEGGYFHIET | |
| | DAQTNEGIVTLIKEVDYEEMKNLDFSVIVANKAAFHKSI | |
| | RSKYKPTPIPIKVKVKNVKEGIHFKSSVISIYVSESMDRSS | |
| | KGQIIGNFQAFDEDTGLPAHARYVKLEDRDNWISVDSVT | |
| | SEIKLAKLPDFESRYVQNGTYTVKIVAISEDYPRKTITGT | |
| | VLINVEDINDNCPTLIEPVQTICHDAEYVNVTAEDLDGHP | |
| | NSGPFSFSVIDKPPGMAEKWKIARQESTSVLLQQSEKKL | |
| | GRSEIQFLISDNQGFSCPEKQVLTLTVCECLHGSGCREAQ | |
| | HDSYVG | |
| 14 | MARSPGDRCALLLLVQLLAVVCLDFGNGLHLEVFSPRN | Mus musculus |
| | EGKPFPKHTHLVRQKRAWITAPVALREGEDLSRKNPIAKI | DSG2 protein |
| | HSDLAEEKGIKITYKYTGKGITEPPFGIFVFDRNTGELNIT | |
| | SILDREETPYFLLTGYALDSRGNNLEKPLELRIKVLDIND | |
| | NEPVFTQEVFVGSIEELSAAHTLVMKITATDADDPETLN | |
| | AKVSYRIVSQEPANSHMFYLNKDTGEIYTTSFTLDREEHS | |
| | SYSLTVEARDGNGQITDKPVQQAQVQIRILDVNDNIPVV | |
| | ENKMYEGTVEENQVNVEVMRIKVTDADEVGSDNWLAN | |
| | FTFASGNEGGYFHIETDTQTNEGIVTLVKEVDYEEMKKL | |
| | DLSIIVTNKAAFHKSILSKYKATPIPITVKVKNVVEGIHFK | |
| | SSVVSFRASEAMDRSSLSRSIGNFQVFDEDTGQAAKVTY | |
| | VKVQDTDNWVSVDSVTSEIKLVKIPDFESRYVQNGTYT | |
| | AKVVAISKEHPQKTITGTIVITVEDVNDNCPVLVDSVRSV | |
| | CEDEPYVNVTAEDLDGAQNSAPFSFSIIDQPPGTAQKWKI | |
| | THQESTSVLLQQSERKRGRSEIPFLISDSQGFSCPERQVLQ | |
| | LTVCECLKGGGCVAAQYDNYVGLGPAAIALMILALLLL | |
| | LLVPLLLLICHCGGGAKGFTPIPGTIEMLHPWNNEGAPPE | |
| | DKVVPSLLVADHAESSAVRGGVGGAMLKEGMMKGSSS | |
| | ASVTKGQHELSEVDGRWEEHRSLLTAGATHHVRTAGTI | |
| | AANEAVRTRATGSSRDMSGARGAVAVNEEFLRSYFTEK | |
| | AASYNGEDDLHMAKDCLLVYSQEDTASLRGSVGCCSFI | |
| | EGELDDLFLDDLGLKFKTLAEVCLGRKIDLDVDIEQRQK | |
| | PVREASVSAASGSHYEQAVTSSESAYSSNTGFPAPKPLHE | |
| | VHTEKVTQEIVTESSVSSRQSQKVVPPPDPVASGNIIVTET | |
| | SYAKGSAVPPSTVLLAPRQPQSLIVTERVYAPTSTLVDQH | |
| | YANEEKVLVTERVIQPNGGIPKPLEVTQHLKDAQYVMV | |
| | RERESILAPSSGVQPTLAMPSVAAGGQNVTVTERILTPAS | |
| | TLQSSYQIPSETSITARNTVLSSVGSIGPLPNLDLEESDR | |
| | PNSTITTSSTRVTKHSTMQHSYS | |
| 15 | MARSPSGRCTLLLLLQLLAVVCLDFGDGLHLEVESTRGE | Rattus |
| | DRLFTKRTHLVRQKRAWITAPVALREGEDLSKKNPIAKI | norvegicus |
| | HSDLAEERGIKITYKYTGKGITEPPFGIFVFDRNTGDLNIT | DSG2 protein |
| | SILDREETPFFLLTGYALDSRGNNLEKPLELRIKVLDINDN | |
| | EPVFTQEVFVGSIEELSAAHTLVMKITATDADDPQTLNS | |
| | KISYRIVSQEPANTPVFYLNKDTGEIYTTSFALDREEHSSY | |
| | SLTVEARDGNGQITDKPVQQAQVQIRILDVNDNIPVVEY | |
| | GTYEGVVEENQVNVEVMRIKVTDADEVGSDNWLANFT | |
| | FASGNEGGYFHIETDTQTNEGIVTLVKELDYEEMKKLDL | |
| | SILVTNKAAFHKSVVSKYKATPIPITVKVKNVVEGIHFKS | |
| | STVTFRASEAMDRSSLSRSIGKFQVFDEDTGKAADVTYV | |
| | KVQDTDNWVSVDSETSEIKLVKIPDFESRHVQNGTYTVK | |
| | VVAISKGHPQKTVTGTILINVEDVNDNCPVLVDSVRSVC | |
| | ADAPYVNVTAEDLDGPRNSGPFTFSIIDQPPGTAQKWKIT | |
| | HQESTSVLLQPSERKLGRSEIPFLISDSQGFSCLERQVLQL | |
| | TVCECLEGGGCVAALYDNYVGLGPAAIALMILALLLLLL | |
| | VPLLLLMCHCGEGAKGFAPIPGTIEMLHPWNNEGAPPED | |
| | KVVPSLLVTDHVENSGVRGGGGGGGGGVGGVKLKEGV | |
| | SSSASITKGQHELIEVDGRWEEHRSLLTSGLTHHVATAGS | |
| | IAANDTTIRTTRATGASRDMSGARGAVAMNEELLRSYFT | |
| | EKAASYAGEDDFHLAKDCLLVYSQEDTDSLRGSVGCCS | |
| | FIEGELDDLFLDDLGPKFKTLAEVCLGRKIDMDVDIEQR | |
| | QKPVRETSVNAAPGSHYTQVTASSESTYSSNSGFQAPKP | |
| | LHGAHTEKVTQEVVTESSVSSRQSQKVVPPPNPVASGNII | |
| | VTETSYAAGSAVPPSTVILAPRQPQSLIVTERVYAPASTL | |
| | VDQHYANEENVLVTERVIQPNGGIPKPLEVTQHLNDAQ | |
| | YVMVRERESILAPSSGVQPTLAMPSVAAGGQNVTVTER | |
| | VLTPASTLQSSYQIPSEASIKARKTVLSSVGGMGPLPNLD | |
| | LEESGPPNSTVTTSSTRVTKHSTVQHSYS | |
| 16 | MARSPGRAYALLLLLICFNVGSGLYLQVSSTRNENKLLP | Macaca mulatta |
| | KHPHLVRQKRAWITAPVALREGEDLSKKNPIAKIHSDLT | DSG2 protein |
| | EERGLKVTYKYTGKGITEPPFGIFVENKDTGELNITSILDR | |
| | EETPFFLLTGYALDERGNNVEKPLELRIKVLDINDNEPVF | |
| | TQDVFVGSVEELNTLVMKINATDADEPNTLNSKISYRIVS | |
| | LEPAYPPVFYLNKDTGEIYTTSVTLDREEHSSYTLTVEAR | |
| | DGNGEVTDKPVKQAQVQIRILDVNDNIPVVENKVLEGM | |
| | VEENQVNVEVTRIKVFDADEIGSDNWLANFTFASGNEG | |
| | GYFHIETDAQTNEGIVTLIKEVDYEEMKNLDFSVIVTNK | |
| | AAFHKSIRSKYKPTPIPIKVKVKNVKEGIHFKSSVISTYVS | |
| | ESMDKSSKGQIIGKFQAFDEDTGLLARARYVKLEDRDN | |
| | WISLDSVTSEIKLAKLPDFESRYVQNGTYTVKIVAISEDY | |
| | PRKTITGTVLIYVEDINDNCPTLIEPVQTICNDAQYVNVT | |
| | AEDLDGHPNSGPFSFSVIDKPPGMAEKWKIVHQESTSVL | |
| | LQQSEKKLGRSEIQFLISDNQGFSCPEKQMLTLTVCECLH | |
| | GSGCREAQRDSYVGLGPAAIALMILAFLLLLLVPLLLLM | |
| | CHCGKGGKGFTPIPGTIEMLHPWNNEGAPPEDKVVPSFL | |
| | PVDQGESLGGRNGVGGMAKEATMKGSSSASFAKGQHE | |
| | MSEMDGRWEEYRSLLSGRATQVTGATGAIMTTETTKTT | |
| | RATGASRDMAGAQAAAVALNEEFLRNYFSDKAASYTEE | |
| | DENHTAKDCLLVYSQEETESLNASIGCCSFIEGELDDRFL | |
| | DDLGLKFKTLAEVCLGQKIDINMEIEQRQKPATETSMNT | |
| | ASRSAYEQTMVNSENTYSSGSSFPVPKSLQEANAEKVTQ | |
| | EIVTERSVSSRQAQKVATPLPDPVASRNIIATETSYVTGST | |
| | MPPSTVILGPRQPQSLIVTERVYAPASALVDQPYANEGT | |
| | VVVTERVIQPHGGGSNPLEGTQHLQDAPYVMVRERESF | |
| | LAPSSGVQPTLAMPNIAVGQNVTVTERVLAPASTLQSSY | |
| | QIPTENSVKARNTMVSGAGVPGPLPDFGLEESGHSNSTIT | |
| | TSSTRVTKHSTVQHSYS | |
| 17 | MARSAEGAALLVLICFYFGNGLHLEVLNARNGNTLLPK | Canis lupus |
| | HRHLVRQKRAWITAPVALREGEDLSKRNPIAKIHSDVAE | familiaris DSG2 |
| | ERGLKITYKYTGKGITEPPFGVFVFNKDTGELNVTKILDR | protein |
| | EETPSFLLIGYALDEKGNNLEKPLELRIKVLDINDNEPVFT | |
| | QDVFAGSIEELSAADTLVMKISATDADEPNTLNSKISYRI | |
| | VSQEPTYPPVFYLNKDTGEIYTTSFTLDREEHSSYTLTVE | |
| | ARDGNGQITDKPVKQAQVQIRILDVNDNIPVVKNEMNE | |
| | GVVEENQANVEVMRIKVFDADEIGSDNWLANFTFVSGN | |
| | EMGYFHIETDTQTNEGIVTLIKEVDYEEVKNLDFSIVVTN | |
| | KAAFHKSVKNKYKPTSIPFKIKVKNVKEGIHFKSSTVSVH | |
| | VSESMDKSSQSQIIGKFQVFDEDTGQQARVKYAKLEDID | |
| | NWISVDSVTSEIRLVKIPDFESRYVQNGTYTAKILAISESY | |
| | PRKTITGTVVITVEDINDNCPTLVDPVQNICDDEQYVNVT | |
| | AEDLDGPQNSGPFSFSIIDKPAGMAEKWQIVHQESTSVLL | |
| | QQKEQKLGRSEIQFLISDSQGFSCSEKQVLQLTVCKCLDG | |
| | SGCVEPLRGTYVGLGPAAIALIIFALLLLLLVPLLLLMCH | |
| | CGEGAKGFTPIPGTIEMLHPWNNEGAPPEDKVVPLHLTA | |
| | DHGESVAADIRLGVVTTKETTKESSSALIKDHHEMSEID | |
| | GRWEEHRSLISGGVTQVTGTTGANIGAEALRITKATGSS | |
| | RDLAGAQAAAAAVNEEFLKSYFTEKAASYTDEDDIHTG | |
| | KDCLLVYSQEDTASLHGSIGCCSFIEGELDDGFLDDLGFK | |
| | FRTLAEICLGQKIDMDVEMEQRQKHAGESSMKAASHSF | |
| | SEQTNINSENAYFSGSSFQVPKPLDEANAEKVTQEIVTES | |
| | SVSSRQGHKVATLLPDPLASGNVVVTETSYGTGSTMPPS | |
| | TVVLGPGQSQALIVTERVYAPAPPLVEQRYANDTNVMV | |
| | TETIIQPNGGFPGPLEGTQHLPDGRYVVVRERESFLAPRS | |
| | GAQPTLAMPGVAVGQNMTVTERVQTPASSYWIPAQTSV | |
| | MARKTEVSGAGVRGPLPNCSLEESGHSNSMLTGHSNSTL | |
| | TASSTRVSKHSIVQHSYS | |
| 18 | MARRISPVVAFLLCFGLSHFFEAEARLQHSVALHRQKRE | Danio rerio |
| | WIVPPQILEENVDYTKKDFIAKIRSDKEVAHMKYLRYSL | DSG2 protein |
| | RGVGADQEPFNLFVVNPETGYVRITGILDRESISQYNLSG | |
| | IALFTDGSIAENDIGLRIKVKDQNDNAPVFGVMNPGAVD | |
| | ELSAVGTEVMRLNCFDADEPGNPNSQIKYEIVDQQPAGQ | |
| | SMFTVENNRRVVVANPNLDRETVDQYVLLVKASDLNG | |
| | APGGNAATGTVTIKINDVNDNVPTLGGPYEASIEENTEK | |
| | VEVMRLKVSDLHLKGTDNWEGDCYIASGNEAGYFSIHM | |
| | DPKTNEAVLMLDKAVDYEDVKDLNLGIGVANKAPFHPS | |
| | VSGGSQGATISFGGSGGGAGSGAAGGAGAMGGASGSG | |
| | GGTGASSWSSSGVPLYNVNIKVKNQPEGPKFFPGTKAIPI | |
| | SEGKAFDSTEIIARYPAIDTDTGKEATNVKYIKSSDPDNW | |
| | LTIDEKTGAIRMNKAPDRESKYLVNGTYYAKVLSITQDL | |
| | PSKTATGTIAIQVEDFNDHCPTLISNVQTLCTDKDAVLVT | |
| | AKDEDAPPNGAPFDFNIVSEGTAGKWTVEYMNDTTAIF | |
| | RTHEKLWPGPHELMVEVTDQQGLKCPEPQKLQVDVCTC | |
| | KNQGGCDRTATGDAKKGSRLGPAGIGLLLLALLALLLIP | |
| | LLLLLCTCGMTGAFTDMPFETKVHLISSNTEGQGEDRDV | |
| | PLMCPPSNVDGMGFMTKDYMAVGAMHSAGLGLGVGA | |
| | GVGAAGFLESTSTMGGRGYNEMELDYMNSIGRNNAYSS | |
| | RDMAGDFDGMALSDGYLCEYYSQKSRVVDGFGKDDPM | |
| | VYDYEGKGSPVGSVGCCSLLEDQNDLEFLNDLGPKFTTL | |
| | ADICGGKKTEIPAPAPAPLPPPPKPVVDRSEVVSSTTNILN | |
| | TGNIATNRVNTVNVASNMATASSTRVENVLVTDNRPTM | |
| | ITSVHPAPTLLVQPQPMYYMVEQQPSTVLVAERPAMTQ | |
| | GMYVLNSGPVAEGMVVQGGNIAANTLTRGERMVLVFR | |
| | GGPAQALNNGMLHTSNLSGSQLLLVDGGATSGQVLQGT | |
| | IQRGVAGSQGLMFVDGQGGQVIQGSINNGISTHGGSQNV | |
| | FYVENKGGSSVVQGGLQMGKASTAGSLIGDVGIGGSSV | |
| | KITQNPSSHKVVVQERKVVTTQSVK | |
| 30 | MARTRDRVRLLLLLICFNVGSGLHLQVLSTRNENKLLPK | Human DSG2 |
| | HPHLVRQKRAWITAPVALREGEDLSKKNPIAKIHSDLAE | protein |
| | ERGLKITYKYTGKGITEPPFGIFVFNKDTGELNVTSILDRE | CAA81226 |
| | ETPFFLLTGYALDARGNNVEKPLELRIKVLDINDNEPVFT | |
| | QDVFVGSVEELSAAHTLVMKINATDADEPNTLNSKISYR | |
| | IVSLEPAYPPVFYLNKDTGEIYTTSVTLDREEHSSYTLTV | |
| | EARDGNGEVTDKPVKQAQVQIRILDVNDNIPVVENKVL | |
| | EGMVEENQVNVEVTRIKVFDADEIGSDNWLANFTFASG | |
| | NEGGYFHIETDAQTNEGIVTLIKEVDYEEMKNLDFSVIV | |
| | ANKAAFHKSIRSKYKPTPIPIKVKVKNVKEGIHFKSSVISI | |
| | YVSESMDRSSKGQIIGNFQAFDEDTGLPAHARYVKLEDR | |
| | DNWISVDSVTSEIKLAKLPDFESRYVQNGTYTVKIVAISE | |
| | DYPRKTITGTVLINVEDINDNCPTLIEPVQTICHDAEYVN | |
| | VTAEDLDGHPNSGPFSFSVIDKPPGMAEKWKIARQESTS | |
| | VLLQQSEKKLGRSEIQFLISDNQGFSCPEKQVLTLTVCEV | |
| | LHGSGCREAQHDSYVGLGPAAIALMILAFLLLLLVPLLL | |
| | LMCHCGKGAKAFTPIPGTIEMLHPWNNEGAPPEDKVVPS | |
| | FLPVDQGGSLVGRNGVGGMAKEATMKGSSSASIVKGQH | |
| | EMSEMDGRWEEHRSLLSGRATQFTGATGAIMTTETTKT | |
| | ARATGASRDMAGAQAAAVALNEEFLRNYFTDKAASYT | |
| | EEDENHTAKDCLLVYSQEETESLNASIGCCSFIEGELDDR | |
| | FLDDLGLKFKTLAEVCLGQKIDINKEIEQRQKPATETSM | |
| | NTASHSLCEQTMVNSENTYSSGSSFPVPKSLQEANAEKV | |
| | TQEIVTERSVSSRQAQKVATPLPDPMASRNVIATETSYVT | |
| | GSTMPPTTVILGPSQPQSLIVTERVYAPASTLVDQPYANE | |
| | GTVVVTERVIQPHGGGSNPLEGTQHLQDVPYVMVRERE | |
| | SFLAPSSGVQPTLAMPNIAVGQNVTVTERVLAPASTLQS | |
| | SYQIPTENSMTARNTTVSGAGVPGPLPDFGLEESGHSNST | |
| | ITTSSTRVTKHSTVQHSYS | |

The DSG2 fusion polypeptides of the present disclosure may include one or more domains of the extracellular region of DSG2. The domains from the extracellular region of DSG2 may include repeats of one or more of the EC domains or EA domains, in tandem or in a mixed order. For example, DSG2 fusion polypeptide may include 2, 3, or more repeats of EC1, EC2, EC3, EC4 or EA domains. When more than one domain and/or more than one repeat of a domain of the extracellular region of DSG2 is present, the domains may be operably linked via a linker described herein.

In some embodiments, the DSG2 fusion polypeptide may include two domains of the extracellular region of DSG2. Non limiting examples domains of extracellular region of DSG2 present in the fusion polypeptides of the present disclosure include, EC1EC2, EC1EC3, EC1EC4, EC1EA, EC2EC1, EC2EC3, EC2EC4, EC2EA, EC3EC1, EC3EC2, EC3EC4, EC3EA, EC4EC1, EC4EC2, EC4EC3, EC4EA, EAEC1, EAEC2, EAEC3, and/or EAEC4.

In some embodiments, the DSG2 fusion polypeptide may include three domains of the extracellular region of DSG2. Non limiting examples domains of extracellular region of DSG2 present in the fusion polypeptides of the present disclosure include, EC1EC2EC3, EC1EC2EC4, EC1EC2EA, EC1EC3EC2, EC1EC3EC4, EC1EC3EA, EC1EC4EC2, EC1EC4EC3, EC1EC4EA, EC1EAEC2, EC1EAEC3, EC1EAEC4, EC2EC1EC3, EC2EC1EC4, EC2EC1EA, EC2EC3EC1, EC2EC3EC4, EC2EC3EA, EC2EC4EC1, EC2EC4EC3, EC2EC4EA, EC2EAEC1, EC2EAEC3, EC2EAEC4, EC3EC1EC2, EC3EC1EC4, EC3EC1EA, EC3EC2EC1, EC3EC2EC4, EC3EC2EA, EC3EC4EC1, EC3EC4EC2, EC3EC4EA, EC3EAEC1, EC3EAEC2, EC3EAEC4, EC4EC1EC2, EC4EC1EC3, EC4EC1EA, EC4EC2EC1, EC4EC2EC3, EC4EC2EA, EC4EC3EC1, EC4EC3EC2, EC4EC3EA, EC4EAEC1, EC4EAEC2, EC4EAEC3, EAEC1EC2, EAEC1EC3, EAEC1EC4, EAEC2EC1, EAEC2EC3, EAEC2EC4, EAEC3EC1, EAEC3EC2, EAEC3EC4, EAEC4EC1, EAEC4EC2, and/or EAEC4EC3.

In some embodiments, the DSG2 fusion polypeptide may include four domains of the extracellular region of DSG2. Non limiting examples domains of extracellular region of DSG2 present in the fusion polypeptides of the present disclosure include, EC1EC2EC3EC4, EC1EC2EC3EA, EC1EC2EC4EC3, EC1EC2EC4EA, EC1EC2EAEC3, EC1EC2EAEC4, EC1EC3EC2EC4, EC1EC3EC2EA, EC1EC3EC4EC2, EC1EC3EC4EA, EC1EC3EAEC2, EC1EC3EAEC4, EC1EC4EC2EC3, EC1EC4EC2EA, EC1EC4EC3EC2, EC1EC4EC3EA, EC1EC4EAEC2, EC1EC4EAEC3, EC1EAEC2EC3, EC1EAEC2EC4, EC1EAEC3EC2, EC1EAEC3EC4, EC1EAEC4EC2, EC1EAEC4EC3, EC2EC1EC3EC4, EC2EC1EC3EA, EC2EC1EC4EC3, EC2EC1EC4EA, EC2EC1EAEC3, EC2EC1EAEC4, EC2EC3EC1EC4, EC2EC3EC1EA, EC2EC3EC4EC1, EC2EC3EC4EA, EC2EC3EAEC1, EC2EC3EAEC4, EC2EC4EC1EC3, EC2EC4EC1EA, EC2EC4EC3EC1, EC2EC4EC3EA, EC2EC4EAEC1, EC2EC4EAEC3, EC2EAEC1EC3, EC2EAEC1EC4, EC2EAEC3EC1, EC2EAEC3EC4, EC2EAEC4EC1, EC2EAEC4EC3, EC3EC1EC2EC4, EC3EC1EC2EA, EC3EC1EC4EC2, EC3EC1EC4EA, EC3EC1EAEC2, EC3EC1EAEC4, EC3EC2EC1EC4, EC3EC2EC1EA, EC3EC2EC4EC1, EC3EC2EC4EA, EC3EC2EAEC1, EC3EC2EAEC4, EC3EC4EC1EC2, EC3EC4EC1EA, EC3EC4EC2EC1, EC3EC4EC2EA, EC3EC4EAEC1, EC3EC4EAEC2, EC3EAEC1EC2, EC3EAEC1EC4, EC3EAEC2EC1, EC3EAEC2EC4, EC3EAEC4EC1, EC3EAEC4EC2, EC4EC1EC2EC3, EC4EC1EC2EA, EC4EC1EC3EC2, EC4EC1EC3EA, EC4EC1EAEC2, EC4EC1EAEC3, EC4EC2EC1EC3, EC4EC2EC1EA, EC4EC2EC3EC1, EC4EC2EC3EA, EC4EC2EAEC1, EC4EC2EAEC3, EC4EC3EC1EC2, EC4EC3EC1EA, EC4EC3EC2EC1, EC4EC3EC2EA, EC4EC3EAEC1, EC4EC3EAEC2, EC4EAEC1EC2, EC4EAEC1EC3, EC4EAEC2EC1, EC4EAEC2EC3, EC4EAEC3EC1, EC4EAEC3EC2, EAEC1EC2EC3, EAEC1EC2EC4, EAEC1EC3EC2, EAEC1EC3EC4, EAEC1EC4EC2, EAEC1EC4EC3, EAEC2EC1EC3, EAEC2EC1EC4, EAEC2EC3EC1, EAEC2EC3EC4, EAEC2EC4EC1, EAEC2EC4EC3, EAEC3EC1EC2, EAEC3EC1EC4, EAEC3EC2EC1, EAEC3EC2EC4, EAEC3EC4EC1, EAEC3EC4EC2, EAEC4EC1EC2, EAEC4EC1EC3, EAEC4EC2EC1, EAEC4EC2EC3, EAEC4EC3EC1, and/or EAEC4EC3EC2.

In some embodiments, the DSG2 fusion polypeptide may include five domains of the extracellular region of DSG2. Non limiting examples domains of extracellular region of DSG2 present in the fusion polypeptides of the present disclosure include EC1EC2EC3EC4EA, EC1EC2EC3EAEC4, EC1EC2EC4EC3EA, EC1EC2EC4EAEC3, EC1EC2EAEC3EC4, EC1EC2EAEC4EC3, EC1EC3EC2EC4EA, EC1EC3EC2EAEC4, EC1EC3EC4EC2EA, EC1EC3EC4EAEC2, EC1EC3EAEC2EC4, EC1EC3EAEC4EC2, EC1EC4EC2EC3EA, EC1EC4EC2EAEC3, EC1EC4EC3EC2EA, EC1EC4EC3EAEC2, EC1EC4EAEC2EC3, EC1EC4EAEC3EC2, EC1EAEC2EC3EC4, EC1EAEC2EC4EC3, EC1EAEC3EC2EC4, EC1EAEC3EC4EC2, EC1EAEC4EC2EC3, EC1EAEC4EC3EC2, EC2EC1EC3EC4EA, EC2EC1EC3EAEC4, EC2EC1EC4EC3EA, EC2EC1EC4EAEC3, EC2EC1EAEC3EC4, EC2EC1EAEC4EC3, EC2EC3EC1EC4EA, EC2EC3EC1EAEC4, EC2EC3EC4EC1EA, EC2EC3EC4EAEC1, EC2EC3EAEC1EC4, EC2EC3EAEC4EC1, EC2EC4EC1EC3EA, EC2EC4EC1EAEC3, EC2EC4EC3EC1EA, EC2EC4EC3EAEC1, EC2EC4EAEC1EC3, EC2EC4EAEC3EC1, EC2EAEC1EC3EC4, EC2EAEC1EC4EC3, EC2EAEC3EC1EC4, EC2EAEC3EC4EC1, EC2EAEC4EC1EC3, EC2EAEC4EC3EC1, EC3EC1EC2EC4EA, EC3EC1EC2EAEC4, EC3EC1EC4EC2EA, EC3EC1EC4EAEC2, EC3EC1EAEC2EC4, EC3EC1EAEC4EC2, EC3EC2EC1EC4EA, EC3EC2EC1EAEC4, EC3EC2EC4EC1EA, EC3EC2EC4EAEC1, EC3EC2EAEC1EC4, EC3EC2EAEC4EC1, EC3EC4EC1EC2EA, EC3EC4EC1EAEC2, EC3EC4EC2EC1EA, EC3EC4EC2EAEC1, EC3EC4EAEC1EC2, EC3EC4EAEC2EC1, EC3EAEC1EC2EC4, EC3EAEC1EC4EC2, EC3EAEC2EC1EC4, EC3EAEC2EC4EC1, EC3EAEC4EC1EC2, EC3EAEC4EC2EC1, EC4EC1EC2EC3EA, EC4EC1EC2EAEC3, EC4EC1EC3EC2EA, EC4EC1EC3EAEC2, EC4EC1EAEC2EC3, EC4EC1EAEC3EC2, EC4EC2EC1EC3EA, EC4EC2EC1EAEC3, EC4EC2EC3EC1EA, EC4EC2EC3EAEC1, EC4EC2EAEC1EC3, EC4EC2EAEC3EC1, EC4EC3EC1EC2EA, EC4EC3EC1EAEC2, EC4EC3EC2EC1EA, EC4EC3EC2EAEC1, EC4EC3EAEC1EC2, EC4EC3EAEC2EC1, EC4EAEC1EC2EC3, EC4EAEC1EC3EC2, EC4EAEC2EC1EC3, EC4EAEC2EC3EC1, EC4EAEC3EC1EC2, EC4EAEC3EC2EC1, EAEC1EC2EC3EC4, EAEC1EC2EC4EC3, EAEC1EC3EC2EC4, EAEC1EC3EC4EC2, EAEC1EC4EC2EC3, EAEC1EC4EC3EC2, EAEC2EC1EC3EC4, EAEC2EC1EC4EC3, EAEC2EC3EC1EC4, EAEC2EC3EC4EC1, EAEC2EC4EC1EC3, EAEC2EC4EC3EC1, EAEC3EC1EC2EC4, EAEC3EC1EC4EC2, EAEC3EC2EC1EC4, EAEC3EC2EC4EC1, EAEC3EC4EC1EC2, EAEC3EC4EC2EC1, EAEC4EC1EC2EC3, EAEC4EC1EC3EC2, EAEC4EC2EC1EC3, EAEC4EC2EC3EC1, EAEC4EC3EC1EC2, and/or EAEC4EC3EC2EC1.

Non-limiting examples of a portion of the DSG2 protein as well as possible configurations present in the DSG2 fusion polypeptide are provided in Table 2. Any of the DSG2 domains described in Table 2 may be operably linked to another domain within the fusion polypeptide or to another DSG2 domain using any of the linker provided herein. The compositions of the disclosure may include a portion or a fragment of any of the domains described in Table 2. The domains or combination of domains of SEQ ID NO: 1 or SEQ ID NO: 3 included in the polypeptides of the disclosure may be extended by 1, 2, 3, 4, 5, 6, 7, 8, 9, 10, 11, 12, 13, 14, 15, 16, 17, 18, 19, 20, 30, 40 or 50 amino acids upstream or downstream of the domains defined in Table 2. In some embodiments, the domains or combination of domains of SEQ ID NO: 1 or SEQ ID NO: 3 included in the polypeptides of the disclosure may be truncated by 1, 2, 3, 4, 5, 6, 7, 8, 9, 10, 11, 12, 13, 14, 15, 16, 17, 18, 19, 20, 30, 40 or 50 amino acids at the N terminus or the C terminus of the domains defined in Table 2. As a non-limiting example, the extracellular region of DSG2 protein may extend from the amino acids spanning from amino acids 50-609 of SEQ ID NO: 1.

TABLE 2
DSG2 domain combination for the DSG2 extracellular region

| DSG2 domain or combination | Amino acids of SEQ ID NO: 1 |
|---|---|
| EC1 | 50-155 |
| EC2 | 151-268 |
| EC3 | 264-384 |
| EC4 | 382-495 |
| EA | 491-609 |
| EC4EA | 382-609 |
| EC3EC4EA | 264-609 |
| EC2EC3EC4EA | 151-609 |
| EC1EC2EC3EC4EA | 50-609 |
| EC1EC2 | 50-268 |
| EC1EC2EC3 | 50-384 |
| EC1EC2EC3EC4 | 50-495 |
| EC2EC3 | 151-384 |
| EC2EC3EC4 | 151-495 |
| EC3EC4 | 264-495 |
| EC1EC3EC4EA | 50-155, 264-609 |
| EC1EC4EA | 50-155, 382-609 |
| EC1EA | 50-155, 491-609 |
| EC1EC2EC4EA | 50-268, 382-609 |
| EC1EC2EC3EA | 50-384, 491-609 |
| EC1EC3 | 50-155, 264-384 |
| EC1EC3EC4EA | 50-155, 264-384, 491-609 |
| EC2EC4EA | 151-268, 491-609 |
| EC2EC4 | 151-268, 382-495 |
| EC3EA | 264-384, 491-609 |

### Immunoglobulin Protein

In some embodiments, DSG2 fusion polypeptides of the present disclosure may include a whole or a portion of an immunoglobulin protein. The immunoglobulin protein may be an IgG, an IgM, an IgA, an IgD or an IgE. In one embodiment, the immunoglobulin protein may be an IgG. Non-limiting examples of IgG may be IgG1, IgG2, IgG3 and/or IgG4.

DSG2 fusion polypeptides may include a region or a portion of an immunoglobulin. Non-limiting examples of a region of an immunoglobulin such as, an Fc region, an Fab region, a heavy chain variable (VH) domain, a heavy chain constant domain, a light chain variable (VL) domain, and/or a light chain constant domain.

DSG2 fusion polypeptides may include one or more Fc regions of an immunoglobulin. In some embodiments, the Fc region may include the first constant region immunoglobulin domain (e.g., CH1) or a portion thereof, and in some cases, part of the hinge. In other aspects, the Fc region excludes the first constant region immunoglobulin domain. Thus, an Fc may refer to the last two constant region immunoglobulin domains (e.g., CH2 and CH3) of IgA, IgD, and IgG, the last three constant region immunoglobulin domains of IgE and IgM, and the flexible hinge N-terminal to these domains. For IgA and IgM, the Fc region may include the J chain. For IgG, the Fc region comprises immunoglobulin domains Cy2 and Cy3 (Cy2 and Cy3) and the lower hinge region between Cy1 (Cy1) and Cy2 (Cy2). In some embodiments, an Fc region refers to a truncated CH1 domain, and CH2 and CH3 of an immunoglobulin. Although the boundaries of the Fc region may vary, the human IgG heavy chain Fc region are typically usually defined to include residues E216 or C226 or P230 to its carboxyl-terminus, wherein the numbering is according to the EU index as in Kabat Antibody Numbering sequence.

In some embodiments, the DSG2 fusion polypeptides may include a human immunoglobulin protein. In some embodiments, the DSG2 fusion polypeptides may include a non-human immunoglobulin protein. The non-human immunoglobulin protein may be a dog, rat, mouse, or primate immunoglobulin protein. Non-limiting examples of the sequences of portions of immunoglobulin are provided in Table 3. In some embodiments, the DSG2 fusion polypeptides may include an immunoglobulin protein or a portion thereof having at least 50%, 55%, 60%, 65%, 70%, 75%, 80%, 85%, 90%, 95%, 99% or 100% identity to any of the sequences in Table 3 or fragments of the sequences in Table 3.

TABLE 3
Sequences of the Immunoglobulin regions

| SEQ | | |
|---|---|---|
| ID | | |
| NO: | Description | Sequence |
| 4 | Human IgG1 | ASTKGPSVFPLAPSSKSTSGGTAALGCLVKDYFPEPVTVSWNSGALTS |
| | Immunoglobulin | GVHTFPAVLQSSGLYSLSSVVTVPSSSLGTQTYICNVNHKPSNTKVDK |
| | heavy chain | KVEPKSCDKTHTCPPCPAPELLGGPSVFLFPPKPKDTLMISRTPEVTCV |
| | constant region | VVDVSHEDPEVKFNWYVDGVEVHNAKTKPREEQYNSTYRVVSVLT |
| | | VLHQDWLNGKEYKCKVSNKALPAPIEKTISKAKGQPREPQVYTLPPS |
| | | RDELTKNQVSLTCLVKGFYPSDIAVEWESNGQPENNYKTTPPVLDSD |
| | | GSFFLYSKLTVDKSRWQQGNVFSCSVMHEALHNHYTQKSLSLSPGK |
| 5 | Human IgG1 | DKTHTCPPCPAPELLGGPSVFLFPPKPKDTLMISRTPEVTCVVVDVSH |
| | Immunoglobulin | EDPEVKFNWYVDGVEVHNAKTKPREEQYNSTYRVVSVLTVLHQDW |
| | heavy chain | LNGKEYKCKVSNKALPAPIEKTISKAKGQPREPQVYTLPPSRDELTKN |
| | constant Fc | QVSLTCLVKGFYPSDIAVEWESNGQPENNYKTTPPVLDSDGSFFLYSK |
| | Domain | LTVDKSRWQQGNVFSCSVMHEALHNHYTQKSLSLSPGK |
| 6 | Human IgG2 | ASTKGPSVFPLAPCSRSTSESTAALGCLVKDYFPEPVTVSWNSGALTS |
| | Immunoglobulin | GVHTFPAVLQSSGLYSLSSVVTVPSSNFGTQTYTCNVDHKPSNTKVD |
| | heavy chain | KTVERKCCVECPPCPAPPVAGPSVFLFPPKPKDTLMISRTPEVTCVVV |
| | constant region | DVSHEDPEVQFNWYVDGVEVHNAKTKPREEQFNSTFRVVSVLTVVH |
| | | QDWLNGKEYKCKVSNKGLPAPIEKTISKTKGQPREPQVYTLPPSREE |
| | | MTKNQVSLTCLVKGFYPSDISVEWESNGQPENNYKTTPPMLDSDGSF |
| | | FLYSKLTVDKSRWQQGNVFSCSVMHEALHNHYTQKSLSLSPGK |
| 7 | Human IgG2 | ERKCCVECPPCPAPPVAGPSVFLFPPKPKDTLMISRTPEVTCVVVDVS |
| | Immunoglobulin | HEDPEVQFNWYVDGVEVHNAKTKPREEQFNSTFRVVSVLTVVHQD |
| | heavy chain | WLNGKEYKCKVSNKGLPAPIEKTISKTKGQPREPQVYTLPPSREEMT |
| | constant Fc | KNQVSLTCLVKGFYPSDISVEWESNGQPENNYKTTPPMLDSDGSFFL |
| | domain | YSKLTVDKSRWQQGNVFSCSVMHEALHNHYTQKSLSLSPGK |
| 8 | Human IgG3 | ASTKGPSVFPLAPCSRSTSGGTAALGCLVKDYFPEPVTVSWNSGALTS |
| | Immunoglobulin | GVHTFPAVLQSSGLYSLSSVVTVPSSSLGTQTYTCNVNHKPSNTKVD |
| | heavy chain | KRVELKTPLGDTTHTCPRCPEPKSCDTPPPCPRCPEPKSCDTPPPCPRC |
| | constant region | PEPKSCDTPPPCPRCPAPELLGGPSVFLFPPKPKDTLMISRTPEVTCVV |
| | | VDVSHEDPEVQFKWYVDGVEVHNAKTKPREEQYNSTFRVVSVLTVL |
| | | HQDWLNGKEYKCKVSNKALPAPIEKTISKTKGQPREPQVYTLPPSRE |
| | | EMTKNQVSLTCLVKGFYPSDIAVEWESSGQPENNYNTTPPMLDSDGS |
| | | FFLYSKLTVDKSRWQQGNIFSCSVMHEALHNRFTQKSLSLSPGK |
| 9 | Human IgG3 | ELKTPLGDTTHTCPRCPEPKSCDTPPPCPRCPEPKSCDTPPPCPRCPEPK |
| | Immunoglobulin | SCDTPPPCPRCPAPELLGGPSVFLFPPKPKDTLMISRTPEVTCVVVDVS |
| | heavy chain Fc | HEDPEVQFKWYVDGVEVHNAKTKPREEQYNSTFRVVSVLTVLHQD |
| | domain | WLNGKEYKCKVSNKALPAPIEKTISKTKGQPREPQVYTLPPSREEMT |
| | | KNQVSLTCLVKGFYPSDIAVEWESSGQPENNYNTTPPMLDSDGSFFL |
| | | YSKLTVDKSRWQQGNIFSCSVMHEALHNRFTQKSLSLSPGK |
| 10 | Human IgG4 | ASTKGPSVFPLAPCSRSTSESTAALGCLVKDYFPEPVTVSWNSGALTS |
| | Immunoglobulin | GVHTFPAVLQSSGLYSLSSVVTVPSSSLGTKTYTCNVDHKPSNTKVD |
| | heavy chain | KRVESKYGPPCPSCPAPEFLGGPSVFLFPPKPKDTLMISRTPEVTCVVV |
| | constant region | DVSQEDPEVQFNWYVDGVEVHNAKTKPREEQFNSTYRVVSVLTVLH |
| | | QDWLNGKEYKCKVSNKGLPSSIEKTISKAKGQPREPQVYTLPPSQEE |
| | | MTKNQVSLTCLVKGFYPSDIAVEWESNGQPENNYKTTPPVLDSDGSF |
| | | FLYSRLTVDKSRWQEGNVFSCSVMHEALHNHYTQKSLSLSLGK |
| 11 | Human IgG4 | ESKYGPPCPSCPAPEFLGGPSVFLFPPKPKDTLMISRTPEVTCVVVDVS |
| | Immunoglobulin | QEDPEVQFNWYVDGVEVHNAKTKPREEQFNSTYRVVSVLTVLHQD |
| | heavy chain Fc | WLNGKEYKCKVSNKGLPSSIEKTISKAKGQPREPQVYTLPPSQEEMT |
| | domain | KNQVSLTCLVKGFYPSDIAVEWESNGQPENNYKTTPPVLDSDGSFFL |
| | | YSRLTVDKSRWQEGNVFSCSVMHEALHNHYTQKSLSLSLGK |
| 19 | Canine | MESVLCWVFLVSILKGVQGEVQLVESGGDLVKPGGSLRLSCVASGFT |
| | immunoglobulin | FSDYGMSWVRQSPGKGLQWVAAVSNRGDTYYADAVKGRFTISRDN |
| | gamma heavy | AKNTLYLQMSSLKAEDTAIYHCVTGVWPRHYYGMDHWGNGTSLFV |
| | chain D | SSASTTAPSVFPLAPSCGSTSGSTVALACLVSGYFPEPVTVSWNSGSLT |
| | | SGVHTFPSVLQSSGLYSLSSTVTVPSSRWPSETFTCNVVHPASNTKVD |
| | | KPVPKESTCKCISPCPVPESLGGPSVFIFPPKPKDILRITRTPEITCVVLD |
| | | LGREDPEVQISWFVDGKEVHTAKTQPREQQFNSTYRVVSVLPIEHQD |
| | | WLTGKEFKCRVNHIGLPSPIERTISKARGQAHQPSVYVLPPSPKELSSS |
| | | DTVTLTCLIKDFFPPEIDVEWQSNGQPEPESKYHTTAPQLDEDGSYFL |
| | | YSKLSVDKSRWQQGDTFTCAVMHEALQNHYTDLSLSHSPGK |
| 20 | Canine | ASTTAPSVFPLAPSCGSTSGSTVALACLVSGYFPEPVTVSWNSGSLTS |
| | immunoglobulin | GVHTFPSVLQSSGLYSLSSTVTVPSSRWPSETFTCNVVHPASNTKVDK |
| | gamma heavy | PVPKESTCKCISPCPVPESLGGPSVFIFPPKPKDILRITRTPEITCVVLDL |
| | chain D Fc | GREDPEVQISWFVDGKEVHTAKTQPREQQFNSTYRVVSVLPIEHQDW |
| | region | LTGKEFKCRVNHIGLPSPIERTISKARGQAHQPSVYVLPPSPKELSSSD |
| | | TVTLTCLIKDFFPPEIDVEWQSNGQPEPESKYHTTAPQLDEDGSYFLY |
| | | SKLSVDKSRWQQGDTFTCAVMHEALQNHYTDLSLSHSPGK |
| 21 | Canine | MESVFCWVFLVVILKGVQGEVQLVESGGDLVKPGGSLRLSCVASGFT |
| | immunoglobulin | FSSYYMHWIRQAPGKGLQRVAHIRGDGRTTHYADAMKGRFTISRDN |
| | gamma heavy | AKNTLYLQMNSLTVEDTAIYYCVKDIYYGVGDYWGQGTLVTVSSAS |
| | chain A | TTAPSVFPLAPSCGSTSGSTVALACLVSGYFPEPVTVSWNSGSLTSGV |
| | | HTFPSVLQSSGLHSLSSMVTVPSSRWPSETFTCNVVHPASNTKVDKPV |
| | | FNECRCTDTPPCPVPEPLGGPSVLIFPPKPKDILRITRTPEVTCVVLDLG |
| | | REDPEVQISWFVDGKEVHTAKTQSREQQFNGTYRVVSVLPIEHQDWL |
| | | TGKEFKCRVNHIDLPSPIERTISKARGRAHKPSVYVLPPSPKELSSSDT |
| | | VSITCLIKDFYPPDIDVEWQSNGQQEPERKHRMTPPQLDEDGSYFLYS |
| | | KLSVDKSRWQQGDPFTCAVMHETLQNHYTDLSLSHSPGK |
| 22 | Canine | ASTTAPSVFPLAPSCGSTSGSTVALACLVSGYFPEPVTVSWNSGSLTS |
| | immunoglobulin | GVHTFPSVLQSSGLHSLSSMVTVPSSRWPSETFTCNVVHPASNTKVD |
| | gamma heavy | KPVFNECRCTDTPPCPVPEPLGGPSVLIFPPKPKDILRITRTPEVTCVVL |
| | chain A Fc | DLGREDPEVQISWFVDGKEVHTAKTQSREQQFNGTYRVVSVLPIEHQ |
| | region | DWLTGKEFKCRVNHIDLPSPIERTISKARGRAHKPSVYVLPPSPKELSS |
| | | SDTVSITCLIKDFYPPDIDVEWQSNGQQEPERKHRMTPPQLDEDGSYF |
| | | LYSKLSVDKSRWQQGDPFTCAVMHETLQNHYTDLSLSHSPGK |
| 23 | Canine | MESVLFWVFLVTILKGVQGEVRLVESGGTLVKPGGSLKLSCVASGFT |
| | immunoglobulin | FRRYSMDWVRQAPGKSLQWVAGINGDGTGTSYSQTVKGRFTISRDN |
| | gamma heavy | AKNTLYLQINSLRAEDSAVYYCAKSWSRNGDLDYWGQGTLVTVSSA |
| | chain B | STTAPSVFPLAPSCGSTSGSTVALACLVSGYFPEPVTVSWNSGSLTSG |
| | | VHTFPSVLQSSGLYSLSSMVTVPSSRWPSETFTCNVAHPASKTKVDKP |
| | | VPKRENGRVPRPPDCPKCPAPEMLGGPSVFIFPPKPKDTLLIARTPEVT |
| | | CVVVDLDPEDPEVQISWFVDGKQMQTAKTQPREEQFNGTYRVVSVL |
| | | PIGHQDWLKGKQFTCKVNNKALPSPIERTISKARGQAHQPSVYVLPPS |
| | | REELSKNTVSLTCLIKDFFPPDIDVEWQSNGQQEPESKYRTTPPQLDE |
| | | DGSYFLYSKLSVDKSRWQRGDTFICAVMHEALHNHYTQESLSHSPG |
| | | K |
| 24 | Canine | ASTTAPSVFPLAPSCGSTSGSTVALACLVSGYFPEPVTVSWNSGSLTS |
| | immunoglobulin | GVHTFPSVLQSSGLYSLSSMVTVPSSRWPSETFTCNVAHPASKTKVD |
| | gamma heavy | KPVPKRENGRVPRPPDCPKCPAPEMLGGPSVFIFPPKPKDTLLIARTPE |
| | chain B Fc | VTCVVVDLDPEDPEVQISWFVDGKQMQTAKTQPREEQFNGTYRVVS |
| | region | VLPIGHQDWLKGKQFTCKVNNKALPSPIERTISKARGQAHQPSVYVL |
| | | PPSREELSKNTVSLTCLIKDFFPPDIDVEWQSNGQQEPESKYRTTPPQL |
| | | DEDGSYFLYSKLSVDKSRWQRGDTFICAVMHEALHNHYTQESLSHSP |
| | | GK |
| 25 | Canine | MESVLYWVFLVAILKGVQGDVQLVESGGDLVKPGGSLRLSCVASGF |
| | immunoglobulin | TFSSCAMSWVRQSPGKGPQWVATIRYDGSDIYYADAVKGRFSISRDN |
| | gamma heavy | AKNTVYLQMNSLRAEDTAVYYCAKAPPYDSYHYGMDYWGPGTSLF |
| | chain C | VSSASTTAPSVFPLAPSCGSQSGSTVALACLVSGYIPEPVTVSWNSVSL |
| | | TSGVHTFPSVLQSSGLYSLSSMVTVPSSRWPSETFTCNVAHPATNTKV |
| | | DKPVAKECECKCNCNNCPCPGCGLLGGPSVFIFPPKPKDILVTARTPT |
| | | VTCVVVDLDPENPEVQISWFVDSKQVQTANTQPREEQSNGTYRVVS |
| | | VLPIGHQDWLSGKQFKCKVNNKALPSPIEEIISKTPGQAHQPNVYVLP |
| | | PSRDEMSKNTVTLTCLVKDFFPPEIDVEWQSNGQQEPESKYRMTPPQ |
| | | LDEDGSYFLYSKLSVDKSRWQRGDTFICAVMHEALHNHYTQISLSHS |
| | | PGK |
| 26 | Canine | ASTTAPSVFPLAPSCGSQSGSTVALACLVSGYIPEPVTVSWNSVSLTSG |
| | immunoglobulin | VHTFPSVLQSSGLYSLSSMVTVPSSRWPSETFTCNVAHPATNTKVDKP |
| | gamma heavy | VAKECECKCNCNNCPCPGCGLLGGPSVFIFPPKPKDILVTARTPTVTC |
| | chain C Fc | VVVDLDPENPEVQISWFVDSKQVQTANTQPREEQSNGTYRVVSVLPI |
| | region | GHQDWLSGKQFKCKVNNKALPSPIEEIISKTPGQAHQPNVYVLPPSRD |
| | | EMSKNTVTLTCLVKDFFPPEIDVEWQSNGQQEPESKYRMTPPQLDED |
| | | GSYFLYSKLSVDKSRWQRGDTFICAVMHEALHNHYTQISLSHSPGK |
| 31 | Human IgG1 | DKTHTCPPCPAPEAAGGPSVFLFPPKPKDTLMISRTPEVTCVVVDVSH |
| | Immunoglobulin | EDPEVKFNWYVDGVEVHNAKTKPREEQYNSTYRVVSVLTVLHQDW |
| | heavy chain | LNGKEYKCKVSNKALGAPIEKTISKAKGQPREPQVYTLPPSRDELTKN |
| | constant Fc | QVSLTCLVKGFYPSDIAVEWESNGQPENNYKTTPPVLDSDGSFFLYSK |
| | domain with | LTVDKSRWQQGNVFSCSVMHEALHNHYTQKSLSLSPGK |
| | L234A/L235A/ | |
| | P329G | |
| | mutations | |
| 32 | Human IgG4 | ESKYGPPCPPCPAPEFEGGPSVFLFPPKPKDTLMISRTPEVTCVVVDVS |
| | Immunoglobulin | QEDPEVQFNWYVDGVEVHNAKTKPREEQFNSTYRVVSVLTVLHQD |
| | heavy chain | WLNGKEYKCKVSNKGLGSSIEKTISKAKGQPREPQVYTLPPSQEEMT |
| | constant Fc | KNQVSLTCLVKGFYPSDIAVEWESNGQPENNYKTTPPVLDSDGSFFL |
| | domain with | YSRLTVDKSRWQEGNVFSCSVMHEALHNHYTQKSLSLSLGK |
| | S228P/L235E/ | |
| | P329G | |
| | mutations | |

In some embodiments, the DSG2 fusion polypeptides may include amino acids 100 to 330 of the heavy chain constant region of IgG1 (GenBank Accession No. P01857.1; SEQ ID NO: 4). In some embodiments, the DSG2 fusion polypeptides may include amino acids 104-330 of the heavy chain constant region of IgG1 (SEQ ID NO: 4) which is herein provided as SEQ ID NO: 5, which represents the Fc region of the IgG1 heavy chain constant region. In some embodiments, the DSG2 fusion polypeptides may include a variant of SEQ ID NO: 5, which is designated P329G LALA (using the Eu amino acid numbering nomenclature). This variant contains L234A/L235A/P329G mutations in the Fc region of IgG1 and is provided herein as SEQ ID NO: 31. The “P329G LALA variant” has been shown to reduce FcγR and C1q interactions of the immunoglobulin and therefore it minimizes the immune reactions triggered by the expression of DSG2 fusion polypeptides in vivo (Schlothauer, et al. Protein Eng. Des. Sel. 2016, 29(10), 457-466, incorporated herein by reference in its entirety). In some alternative embodiments, the IgG1 protein does not include the L234A, L235A and P329G mutations.

In some embodiments, the DSG2 fusion polypeptides may include amino acids 99 to 327 of IgG4 (GenBank Accession No. P01861.1; SEQ ID NO: 10), which is provided herein as SEQ ID NO: 11, representing the Fc region of the IgG4 heavy chain constant region. In some embodiments, the DSG2 fusion polypeptides may include a variant of SEQ ID NO: 11, which includes three mutations designated S228P, L235E and P329G (using the Eu amino acid numbering nomenclature). The sequence of the variant including these three mutations is provided herein as SEQ ID NO: 32.

The human IgG4 S228P/L235E/P329G variant of IgG4 (also referred to herein as “SPLE P329G”) is a variant of IgG4 that has previously demonstrated minimal FcγR binding activity, (see Newman et al. 2001, Clin. Immunol., 98, 164-174; the contents of which are herein incorporated by reference in its entirety). In some embodiments, the IgG4 protein does not include the mutations described above.

### Signal Sequence

Signal sequences (sometimes referred to as signal peptides, targeting signals, target peptides, localization sequences, transit peptides, leader sequences or leader peptides) direct proteins (e.g., the polypeptides of the present disclosure) to their designated cellular and/or extracellular locations. A signal sequence may be a short (5-50 amino acids long) peptide present at the N-terminus of the majority of newly synthesized proteins that are destined towards a particular location. Signal sequences can be recognized by signal recognition particles (SRPs) and cleaved using type I and type II signal peptide peptidases. Signal sequences derived from human proteins can be incorporated as a DSG2 fusion polypeptides of the present disclosure to direct the polypeptides of the disclosure to a particular cellular and/or extracellular location. These signal sequences are experimentally verified and can be cleaved (Zhang et al., Protein Sci. 2004, 13:2819-2824).

In some embodiments, a signal sequence may be located at the N-terminus or C-terminus of the polypeptides of the present disclosure, and may be, although not necessarily, cleaved off the polypeptide to yield a “mature” polypeptide, as discussed herein.

In some examples, a signal sequence may be a secreted signal sequence derived from a naturally secreted protein, and its variant thereof.

In some instances, signal sequences directing the polypeptides of the disclosure to the surface membrane of a target cell may be used. Expression of the polypeptides of the disclosure on the surface of the target cell may be useful to limit the diffusion of the polypeptides of the disclosure to non-target in vivo environments, thereby potentially improving the safety profile of the polypeptides of the disclosure. Additionally, the membrane presentation of the polypeptides of the disclosure may allow for physiologically and qualitative signaling as well as stabilization and recycling of the polypeptide for a longer half-life.

A signal sequence may be a heterogeneous signal sequence from other organisms such as virus, yeast and bacteria, which can direct the polypeptides of the disclosure to a particular cellular site, such as a nucleus (e.g., EP 1209450). Other examples may include Aspartic Protease (NSP24) signal sequences from Trichoderma that can increase secretion of fused protein such as enzymes (e.g., U.S. Pat. No. 8,093,016 to Cervin and Kim), bacterial lipoprotein signal sequences (e.g., PCT publication NO. 1991/09952 to Lau and Rioux), E. coli enterotoxin II signal peptides (e.g., U.S. Pat. No. 6,605,697 to Kwon et al.), E. coli secretion signal sequence (e.g., U.S. patent publication NO. 2016/090404 to Malley et al), a lipase signal sequence from a methylotrophic yeast (e.g., U.S. Pat. No. 8,975,041), and signal peptides for DNases derived from Coryneform bacteria (e.g., U.S. Pat. No. 4,965,197); the contents of each of which are incorporated herein by reference in their entirety.

In some embodiments, the signal peptide may be IgG1 signal peptide or IgG2 signal peptide. In some embodiments, the signal peptide may be mouse IgGκ kappa light chain signal peptide which has the amino acid sequence METDTLLLWVLLLWVPGSTG (SEQ ID NO: 29). This signal peptide is one of the most well characterized signal peptides used to improve transgene expression (Fonseca et al., Vaccine, 2018, 36(20): 2799-2808, incorporated herein by reference in its entirety). The example embodiments of fusion proteins described hereinbelow (with the exception of DSG2FP #1) included the signal peptide of SEQ ID NO: 29 when expressed. The signal peptide was subsequently cleaved during protein production from each fusion protein prior to testing for inhibition of anti-DSG2 antibodies.

### Linkers

In some embodiments, the DSG2 fusion polypeptides of the present disclosure may include at least one linker. The linker may be positioned between one or more regions of the polypeptides of the disclosure. In one embodiment, the linker may be positioned between the whole or a portion of a DSG2 protein and the whole or a portion of an immunoglobulin protein. In one aspect, the linker may be positioned between one or more domains of the DSG2 protein.

In some embodiments, the linker may be a polypeptide. In some embodiments, the linker may comprise combination of amino acid residues. In some embodiments, the linker may comprise about 1-50 amino acid residues. In some embodiments, the linker may comprise about 1, 2, 3, 4, 5, 6, 7, 8, 9, 10, 15, 20, 25, 30, 35, 40, 45 or 50 amino acid residues.

The linkers of the present disclosure may be from about 1 to 100 amino acids in length, which links together any of the domains/regions of the effector module (also known as a peptide linker). The linker may be 1-40 amino acids in length, or 2-30 amino acids in length, or 20-80 amino acids in length, or 50-100 amino acids in length. Linker length may also be optimized depending on the type of configuration of the polypeptide and based on the crystal structure of the polypeptide. In some instances, a shorter linker length may be preferably selected. In some aspects, the peptide linker may be made up of amino acids linked together by peptide bonds, preferably from 1 to 20 amino acids linked by peptide bonds, wherein the amino acids are selected from the 20 naturally occurring amino acids: Glycine (G), Alanine (A), Valine (V), Leucine (L), Isoleucine (I), Serine (S), Cysteine (C), Threonine (T), Methionine (M), Proline (P), Phenylalanine (F), Tyrosine (Y), Tryptophan (W), Histidine (H), Lysine (K), Arginine (R), Aspartate (D), Glutamic acid (E), Asparagine (N), and Glutamine (Q). One or more of these amino acids may be glycosylated, as is understood by those in the art. In some aspects, amino acids of a peptide linker may be selected from Alanine (A), Glycine (G), Proline (P), Asparagine (R), Serine (S), Glutamine (Q) and Lysine (K).

In some embodiments, the linker may be a flexible linker or a rigid linker. Flexible linkers may be composed of small, non-polar (e.g., Gly) or polar (e.g., Ser or Thr) amino acids. The small size of these amino acids provides flexibility, and allows for mobility of the connecting functional domains. The most commonly used flexible linkers have sequences consisting primarily of stretches of Gly and Ser residues (“GS” linker). An example of the most widely used flexible linker has the sequence of (Gly-Gly-Gly-Gly-Ser)n. By adjusting the copy number “n”, the length of this GS linker may be optimized to achieve appropriate separation of the functional domains, or to maintain necessary inter-domain interactions. In some embodiments, linkers may include additional amino acids such as Thr and Ala to maintain flexibility, as well as polar amino acids such as Lys and Glu to improve solubility. In some embodiments, the DSG2 fusion polypeptide may include a flexible linker, such as (Gly)8, consisting of purely of glycine residues. The linker sequence avoided large hydrophobic residues to maintain good solubility in aqueous solutions.

In some embodiments, the linker may be a rigid linker. Non limiting examples of a rigid linker includes a linker with the sequence of (EAAAK)n (n=1-5). In some embodiments, the rigid linker may have a Proline-rich sequence, (XP)n, with X designating any amino acid, preferably Ala, Lys, or Glu.

In some embodiments, the linker may be GGGGGS (SEQ ID NO: 12) or EAAAK (SEQ ID NO: 13). In some embodiments, the linker may be GGGGS (SEQ ID NO: 27). In some embodiments, the linker may be IEGRMD (SEQ ID NO: 28).

### DSG2 Fusion Polypeptides

Certain embodiments of the fusion polypeptides were investigated in the examples described hereinbelow. In the following description, the components are expressed in a format from N terminus to C terminus where components are separated by a semicolon. One of the DSG2 fusion polypeptides investigated was obtained from R&D Systems (rndsystems.com), named “recombinant human desmoglein-2 Fc chimera protein, CF”. This fusion polypeptide is a research tool developed for adhesion of fibroblasts to plate wells and has not been previously investigated in context of inhibiting anti-DSG2 antibodies for therapeutic purposes. The structure of this fusion polypeptide designated herein interchangeably as “Tool Decoy” and “DSG2FP #1” includes an N-terminus with residues Ala49 to Gly608 of GenBank Accession No. CAA81226, representing a full sequence of the extracellular domain of DSG2 (included herein as SEQ ID NO: 30); a linker having the sequence IEGRMD (SEQ ID NO: 28); and residues Pro100 to Lys330 of human IgG1 (SEQ ID NO: 4). In some of the example embodiments of Table 4, the DSG2 extracellular domain includes amino acids 50-609 of the human DSG2 sequence (SEQ ID NO: 1). In some example embodiments of Table 4, the Fc region of the immunoglobulin is IgG1 Fc domain or IgG4Fc domain. In some example embodiments of Table 4, the IgG1 sequence includes amino acids 100-330 or 104-330 of SEQ NO: 4. In some example embodiments of Table 4, the IgG4 sequence includes amino acids 99-327 of SEQ ID NO: 10. In some example embodiments of Table 4, the IgG1 sequence is SEQ ID NO: 31, which, may herein be referred to as the “P329G LALA variant”. In some embodiments, the immunoglobulin protein may be human IgG4 S228P/L235E/P3229G variant of IgG4 (SEQ ID NO: 32). For greater clarity, the mutation designations of the variants described above are with reference to the Eu antibody numbering nomenclature and not positions within the sequences identified in this paragraph. In some embodiments, DSG2 fusion polypeptides may be synthesized as disulfide linked dimers.

In some embodiments, the DSG2 fusion polypeptide may include at least one linker. The linker may be present between any two components of the fusion protein.

Table 4 provides examples of DSG2 fusion polypeptides.

TABLE 4
DSG2 fusion polypeptides

| DSG2 fusion | | |
|---|---|---|
| polypeptide | DSG2 fusion | |
| number | polypeptide | |
| (DSG2FP#) | name | Fusion polypeptide components |
| DSG2FP#1 | Tool decoy | DSG2 extracellular domain (amino acids |
| | | 49-608 of SEQ ID NO: 30); Linker (IEGRMD |
| | | (SEQ ID NO: 28); amino acids 100-330 of |
| | | IgG1 (SEQ ID NO: 4) |
| DSG2FP#2 | DSG2-(IgG1)Fc | DSG2 extracellular domain (amino acids |
| | | 50-609 of SEQ ID NO: 1); amino acids |
| | | 104-330 of IgG1(L234A, L235A, P329G) |
| | | (SEQ ID NO: 31) |
| DSG2FP#3 | DSG2-GGGGS- | DSG2 extracellular domain (amino acids |
| | (IgG1)Fc | 50-609 of SEQ ID NO: 1); Linker GGGGS |
| | | (SEQ ID NO: 12); amino acids 104-330 of |
| | | IgG1 (L234A, L235A, P329G) |
| | | (SEQ ID NO: 31) |
| DSG2FP#4 | DSG2-EAAAK- | DSG2 extracellular domain (amino acids |
| | (IgG1)Fc | 50-609 of SEQ ID NO: 1); Linker EAAAK |
| | | (SEQ ID NO: 13); amino acids 104-330 |
| | | of IgG1 (L234A, L235A, P329G) |
| | | (SEQ ID NO: 31) |
| DSG2FP#5 | (IgG1)Fc-DSG2 | amino acids 104-330 of IgG1 (L234A, |
| | | L235A, P329G; SEQ ID NO: 31); DSG2 |
| | | extracellular domain (amino acids |
| | | 50-609 of SEQ ID NO: 1) |
| DSG2FP#6 | DSG2-(IgG4)Fc | DSG2 extracellular domain (amino acids |
| | | 50-609 of SEQ ID NO: 1); amino acids |
| | | 99-327 of IgG4 (S228P, L235E, P329G) |
| | | (SEQ ID NO: 32) |
| DSG2FP#7 | DSG2-GGGGS- | DSG2 extracellular domain (amino acids |
| | (IgG4)Fc | 50-609 of SEQ ID NO: 1); amino acids |
| | | 99-327 of IgG4 (S228P, L235E, P329G) |
| | | (SEQ ID NO: 32) |

### Therapeutic Agents

In some embodiments, the present disclosure provides therapeutic agents for targeting a condition, a disease, a disorder or a symptom associated with serum anti-DSG2 antibodies. In some embodiments, the therapeutic agent may include an immunoglobulin protein. In some embodiments, the therapeutic agent may be anti-CD20 antibody such as, but not limited to, rituximab. In some embodiments, the therapeutic agents described herein may result in a blockade of autoantibody functions, either by targeting the Fab or Fc fragments. In some embodiments, the therapeutic agents may be intravenously administered immunoglobulins (IVIG). Administering excess IgG, may saturate the FcRn, and thus, all IgG molecules (including the autoantibodies) may be more rapidly cleared. In some embodiments, the therapeutic agent may be an FcRn-blocking monoclonal antibody. In one embodiment, the FcRn-blocking monoclonal antibody may be SYNT001 (Blumberg L J, et al. Sci Adv. 2019 Dec. 18; 5(12):eaax9586. doi: 10.1126/sciadv.aax9586; the contents of which are herein incorporated by reference in its entirety). In some embodiments, the therapeutic agent may be an anti-C5 antibody such as, eculizumab. The activation of the complement system in autoimmune diseases may result in the binding of immune cells to their immune complexes, and the subsequent intracellular signaling events are important for pathogenesis.

Therapeutic agents of the disclosure may be used alone or in combination with the DSG2 fusion polypeptides described herein. When used in combination with the DSG2 fusion polypeptides, the therapeutic agents may be administered prior to, simultaneously with or after the subject has been provided the DSG2 fusion polypeptides. In one embodiment, the therapeutic agent is a CAAR for the treatment of a condition associated with anti-DSG2 antibodies that is not ARVC.

In one embodiment, the compositions of the disclosure may include a combination of therapeutic agents such as, CAAR and the DSG2 fusion polypeptides and may be used for the treatment of a disease such as, but not limited to ARVC, COVID-19, post COVID-19 syndrome, sarcoidosis, dilated cardiomyopathy or any diseases associated with anti-DSG2 antibodies.

### Polynucleotides

In some embodiments, the polypeptides of the present disclosure are encoded by polynucleotides or variants thereof described herein. Exemplary nucleic acids or polynucleotides include, but are not limited to, ribonucleic acids (RNAs), deoxyribonucleic acids (DNAs), threose nucleic acids (TNAs), glycol nucleic acids (GNAs), peptide nucleic acids (PNAs), locked nucleic acids (LNAs, including LNA having a β-D-ribo configuration, α-LNA having an α-L-ribo configuration (a diastereomer of LNA), 2′-amino-LNA having a 2′-amino functionalization, and 2′-amino-a-LNA having a 2′-amino functionalization), ethylene nucleic acids (ENA), cyclohexenyl nucleic acids (CeNA) or hybrids or combinations thereof.

As such, polynucleotides encoding peptides or polypeptides containing substitutions, insertions and/or additions, deletions, and covalent modifications with respect to reference sequences, in particular the polypeptide sequences are disclosed herein. For example, sequence tags or amino acids, such as one or more lysines, can be added to the peptide sequences described herein (e.g., at the N-terminal or C-terminal ends). Sequence tags can be used for peptide purification or localization. Lysines can be used to increase peptide solubility or to allow for biotinylation. Alternatively, amino acid residues located at the carboxy and amino terminal regions of the amino acid sequence of a peptide or protein may optionally be deleted providing for truncated sequences. Certain amino acids (e.g., C-terminal or N-terminal residues) may alternatively be deleted depending on the use of the sequence, as for example, expression of the sequence as part of a larger sequence which is soluble or linked to a solid support.

Once any of the features have been identified or defined as a desired component of a polypeptide to be encoded by a polynucleotide described herein, any of several manipulations and/or modifications of these features may be performed by moving, swapping, inverting, deleting, randomizing, or duplicating. Furthermore, it is understood that manipulation of features may result in the same outcome as a modification to the molecules described herein. For example, a manipulation which involved deleting a domain would result in the alteration of the length of a molecule just as modification of a nucleic acid to encode less than a full-length molecule would.

### III. Pharmaceutical Compositions and Delivery

The fusion polypeptides described herein may be used as therapeutic agents. In some embodiments, the present disclosure provides pharmaceutical compositions comprising at least one pharmaceutically acceptable carrier and a fusion polypeptide.

In some embodiments, compositions are administered to humans, human patients, or subjects. Although the descriptions of pharmaceutical compositions provided herein are principally directed to pharmaceutical compositions which are suitable for administration to humans, it will be understood by the skilled artisan that such compositions are generally suitable for administration to any other animal, e.g., to non-human animals, e.g., non-human mammals. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, humans and/or other primates; mammals, including commercially relevant mammals such as dogs, cattle, pigs, horses, sheep, cats, mice, and/or rats; and/or birds, including commercially relevant birds such as poultry, chickens, ducks, geese, and/or turkeys. As a non-limiting example, compositions of the disclosure may be administered to dogs to treat ARVC.

Provided herein are the fusion polypeptides and pharmaceutical composition thereof which may be used in combination with one or more pharmaceutically acceptable excipients.

In some embodiments, the fusion polypeptides and pharmaceutical compositions of the disclosure may be delivered via the subcutaneous route or via the intravenous route.

In some embodiments, the pharmaceutically acceptable excipients include, but are not limited to, any and all solvents, dispersion media, diluents, or other liquid vehicles, dispersion or suspension aids, surface active agents, isotonic agents, thickening or emulsifying agents, preservatives, solid binders, lubricants, flavoring agents, stabilizers, anti-oxidants, osmolality adjusting agents, pH adjusting agents and the like, as suited to the particular dosage form desired. Various excipients for formulating pharmaceutical compositions and techniques for preparing the composition are known in the art (see Remington: The Science and Practice of Pharmacy, 21” Edition, A. R. Gennaro (Lippincott, Williams & Wilkins, Baltimore, Md., 2006; incorporated herein by reference in its entirety). The use of a conventional excipient medium may be contemplated within the scope of the present disclosure, except insofar as any conventional excipient medium is incompatible with a substance or its derivatives, such as by producing any undesirable biological effect or otherwise interacting in a deleterious manner with any other component(s) of the pharmaceutical composition, its use is contemplated to be within the scope of this disclosure.

In some embodiments, a pharmaceutically acceptable excipient may be at least 95%, at least 96%, at least 97%, at least 98%, at least 99%, or 100% pure. In some embodiments, an excipient is approved for use for humans and for veterinary use. In some embodiments, an excipient may be approved by United States Food and Drug Administration. In some embodiments, an excipient may be of pharmaceutical grade. In some embodiments, an excipient may meet the standards of the United States Pharmacopoeia (USP), the European Pharmacopocia (EP), the British Pharmacopocia, and/or the International Pharmacopocia.

Pharmaceutically acceptable excipients used in the manufacture of pharmaceutical compositions include, but are not limited to, inert diluents, dispersing and/or granulating agents, surface active agents and/or emulsifiers, disintegrating agents, binding agents, preservatives, buffering agents, lubricating agents, and/or oils. Such excipients may optionally be included in pharmaceutical compositions. The composition may also include excipients such as cocoa butter and suppository waxes, coloring agents, coating agents, sweetening, flavoring, and/or perfuming agents.

Exemplary diluents include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and/or combinations thereof.

Exemplary granulating and/or dispersing agents include, but are not limited to, potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked polyvinylpyrrolidone) (crospovidone), sodium carboxymethyl starch (sodium starch glycolate), carboxymethyl cellulose, crosslinked sodium carboxymethyl cellulose (croscarmellose), methylcellulose, pregelatinized starch (starch 1500), microcrystalline starch, water insoluble starch, calcium carboxymethyl cellulose, magnesium aluminum silicate (VEEGUM®), sodium lauryl sulfate, quaternary ammonium compounds, etc., and/or combinations thereof.

Exemplary surface active agents and/or emulsifiers include, but are not limited to, natural emulsifiers (e.g. acacia, agar, alginic acid, sodium alginate, tragacanth, chon-drux, cholesterol, xanthan, pectin, gelatin, egg yolk, casein, wool fat, cholesterol, wax, and lecithin), colloidal clays (e.g. bentonite (aluminum silicate) and VEEGUM® (magnesium aluminum silicate), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxyvinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate (TWEEN®20), polyoxyethylene sorbitan (TWEEN®60), polyoxyethylene sorbitan monooleate (TWEEN®80), sorbitan monopalmitate (SPAN®40), sorbitan monostearate (SPAN®60), sorbitan tristearate (SPAN®65), glyceryl monooleate, sorbitan monooleate (SPAN®80), polyoxyethylene esters (e.g. polyoxyethylene monostearate (MYRJ®45), polyoxyethylene hydrogenated castor oil, polyethoxylated castor oil, polyoxymethylene stearate, and SOLUTOL®), sucrose fatty acid esters, polyethylene glycol fatty acid esters (e.g. CREMOPHOR®), polyoxyethylene ethers, (e.g. polyoxyethylene lauryl ether (BRIJ®30), poly (vinyl-pyrrolidone), diethylene glycol monolaurate, triethanolamine oleate, sodium oleate, potassium oleate, ethyl oleate, oleic acid, ethyl laurate, sodium lauryl sulfate, PLUORINC® F 68, POLOXAMER® 188, cetrimonium bromide, cetylpyridinium chloride, benzalkonium chloride, docusate sodium, etc. and/or combinations thereof.

Exemplary binding agents include, but are not limited to, starch (e.g. cornstarch and starch paste); gelatin; sugars (e.g. sucrose, glucose, dextrose, dextrin, molasses, lactose, lactitol, mannitol); amino acids (e.g., glycine); natural and synthetic gums (e.g. acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (VEEGUM®), and larch arabogalactan); alginates; polyethylene oxide; polyethylene glycol; inorganic calcium salts; silicic acid; polymethacrylates; waxes; water; alcohol; etc.; and combinations thereof.

Exemplary preservatives may include, but are not limited to, antioxidants, chelating agents, antimicrobial preservatives, antifungal preservatives, alcohol preservatives, acidic preservatives, and/or other preservatives. Oxidation is a potential degradation pathway for mRNA, especially for liquid mRNA formulations. In order to prevent oxidation, antioxidants can be added to the formulation. Exemplary antioxidants include, but are not limited to, alpha tocopherol, ascorbic acid, acorbyl palmitate, benzyl alcohol, butylated hydroxyanisole, EDTA, m-cresol, methionine, butylated hydroxytoluene, monothioglycerol, potassium metabisulfite, propionic acid, propyl gallate, sodium ascorbate, sodium bisulfite, sodium metabisulfite, thioglycerol and/or sodium sulfite. Exemplary chelating agents include ethylenediaminetetraacetic acid (EDTA), citric acid monohydrate, disodium edetate, dipotassium edetate, edetic acid, fumaric acid, malic acid, phosphoric acid, sodium edetate, tartaric acid, and/or trisodium edetate. Exemplary antimicrobial preservatives include, but are not limited to, benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and/or thimerosal. Exemplary antifungal preservatives include, but are not limited to, butyl paraben, methyl paraben, ethyl paraben, propyl paraben, benzoic acid, hydroxybenzoic acid, potassium benzoate, potassium sorbate, sodium benzoate, sodium propionate, and/or sorbic acid. Exemplary alcohol preservatives include, but are not limited to, ethanol, polyethylene glycol, phenol, phenolic compounds, bisphenol, chlorobutanol, hydroxybenzoate, and/or phenyl ethyl alcohol. Exemplary acidic preservatives include, but are not limited to, vitamin A, vitamin C, vitamin E, beta-carotene, citric acid, acetic acid, dehydroacetic acid, ascorbic acid, sorbic acid, and/or phytic acid. Other preservatives include, but are not limited to, tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, GLYDANT PLUS®, PHENONIP®, methylparaben, GERMALL®115, GERMABEN®, NEOLONE™, KATHON™, and/or EUXYL®.

In some embodiments, the pH of the pharmaceutical solutions is maintained between pH 5 and pH 8 to improve stability. Exemplary buffers to control pH may include, but are not limited to sodium phosphate, sodium citrate, sodium succinate, histidine (or histidine-HCl), sodium carbonate, and/or sodium malate. In another embodiment, the exemplary buffers listed above may be used with additional monovalent counterions (including, but not limited to potassium). Divalent cations may also be used as buffer counterions; however, these are not preferred due to complex formation and/or mRNA degradation.

Exemplary buffering agents may also include, but are not limited to, citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, etc., and/or combinations thereof.

Exemplary lubricating agents include, but are not limited to, magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, etc., and combinations thereof.

Exemplary oils include, but are not limited to, almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, Litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sca buckthorn, sesame, shea butter, silicone, soybean, sunflower, tea tree, thistle, tsubaki, vetiver, walnut, and wheat germ oils. Exemplary oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and/or combinations thereof.

Excipients such as cocoa butter and suppository waxes, coloring agents, coating agents, sweetening, flavoring, and/or perfuming agents can be present in the composition, according to the judgment of the formulator.

Exemplary additives include physiologically biocompatible buffers (e.g., trimethylamine hydrochloride), addition of chelants (such as, for example, DTPA or DTPA-bisamide) or calcium chelate complexes (as for example calcium DTPA, CaNaDTPA-bisamide), or, optionally, additions of calcium or sodium salts (for example, calcium chloride, calcium ascorbate, calcium gluconate or calcium lactate). In addition, antioxidants and suspending agents can be used.

In some embodiments, the compositions of the present disclosure may be administered by any route which results in a therapeutically effective outcome. These include, but are not limited to enteral (into the intestine), gastroenteral, epidural (into the dura matter), oral (by way of the mouth), transdermal, peridural, intracerebral (into the cerebrum), intracerebroventricular (into the cerebral ventricles), epicutaneous (application onto the skin), intradermal, (into the skin itself), subcutaneous (under the skin), nasal administration (through the nose), intravenous (into a vein), intravenous bolus, intravenous drip, intraarterial (into an artery), intramuscular (into a muscle), intracardiac (into the heart), intraosseous infusion (into the bone marrow), intrathecal (into the spinal canal), intraperitoneal, (infusion or injection into the peritoneum), intravesical infusion, intravitreal, (through the eye), intracavernous injection (into a pathologic cavity) intracavitary (into the base of the penis), intravaginal administration, intrauterine, extra-amniotic administration, transdermal (diffusion through the intact skin for systemic distribution), transmucosal (diffusion through a mucous membrane), transvaginal, insufflation (snorting), sublingual, sublabial, enema, eye drops (onto the conjunctiva), in ear drops, auricular (in or by way of the ear), buccal (directed toward the check), conjunctival, cutaneous, dental (to a tooth or teeth), electroosmosis, endocervical, endosinusial, endotracheal, extracorporeal, hemodialysis, infiltration, interstitial, intraabdominal, intra-amniotic, intra-articular, intrabiliary, intrabronchial, intrabursal, intracartilaginous (within a cartilage), intracaudal (within the cauda equine), intracisternal (within the cisterna magna cerebellomedularis), intracorneal (within the cornea), dental intracornal, intracoronary (within the coronary arteries), intracorporus cavernosum (within the dilatable spaces of the corporus cavernosa of the penis), intradiscal (within a disc), intraductal (within a duct of a gland), intraduodenal (within the duodenum), intradural (within or beneath the dura), intraepidermal (to the epidermis), intraesophageal (to the esophagus), intragastric (within the stomach), intragingival (within the gingivae), intraileal (within the distal portion of the small intestine), intralesional (within or introduced directly to a localized lesion), intraluminal (within a lumen of a tube), intralymphatic (within the lymph), intramedullary (within the marrow cavity of a bone), intrameningeal (within the meninges), intraocular (within the eye), intraovarian (within the ovary), intrapericardial (within the pericardium), intrapleural (within the pleura), intraprostatic (within the prostate gland), intrapulmonary (within the lungs or its bronchi), intrasinal (within the nasal or periorbital sinuses), intraspinal (within the vertebral column), intrasynovial (within the synovial cavity of a joint), intratendinous (within a tendon), intratesticular (within the testicle), intrathecal (within the cerebrospinal fluid at any level of the cerebrospinal axis), intrathoracic (within the thorax), intratubular (within the tubules of an organ), intratumor (within a tumor), intratympanic (within the aurus media), intravascular (within a vessel or vessels), intraventricular (within a ventricle), iontophoresis (by means of electric current where ions of soluble salts migrate into the tissues of the body), irrigation (to bathe or flush open wounds or body cavities), laryngeal (directly upon the larynx), nasogastric (through the nose and into the stomach), occlusive dressing technique, ophthalmic (to the external cyc), oropharyngeal (directly to the mouth and pharynx), parenteral, percutaneous, periarticular, peridural, perineural, periodontal, rectal, respiratory (within the respiratory tract by inhaling orally or nasally for local or systemic effect), retrobulbar (behind the pons or behind the eyeball), intramyocardial (entering the myocardium), soft tissue, subarachnoid, subconjunctival, submucosal, transplacental (through or across the placenta), transtracheal (through the wall of the trachea), transtympanic (across or through the tympanic cavity), ureteral (to the ureter), urethral (to the urethra), vaginal, caudal block, diagnostic, nerve block, biliary perfusion, cardiac perfusion, photopheresis or spinal. In specific embodiments, compositions may be administered in a way which allows them to cross the blood-brain barrier, vascular barrier, or other epithelial barrier.

Therapeutically effective doses will be easily determined by one of skill in the art and will depend on the severity and course of the disease, the patient's health and response to treatment, and the judgment of the treating physician.

### IV. Methods of Use

Provided herein are methods of use of the DSG2 fusion polypeptide compositions of the present disclosure. In one embodiment, compositions of the disclosure may be used to treat diseases associated with DSG2 autoantibodies. In one embodiment, the compositions of the disclosure mitigate cardiotoxicity associated with DSG2 antibodies of any etiology. In some embodiments, DSG2 fusion polypeptide compositions may be used to reduce anti-DSG2 antibodies in a subject. The subject may not have symptoms, diseases or disorders known to generate anti-DSG2 antibodies. In some embodiments, DSG2 fusion polypeptide compositions may be used to reduce serum DSG2 antibody levels. The fusion polypeptides of the disclosure may reduce the DSG2 antibody levels by about 1%, 5%, 10%, 15%, 20%, 25%, 30%, 35%, 40%, 45%, 50%, 55%, 60%, 65%, 70%, 75%, 80%, 85%, 90%, 95%, or 100%.

In some embodiments, any disease associated with the proarrhythmic and/or procardiomyopathie effect of anti-DSG2 antibodies in myocardium may be treated with the DSG2 fusion polypeptides as described herein. Non-limiting examples of such indications include arrhythmogenic right ventricular cardiomyopathy (ARVC), sarcoidosis, dilated cardiomyopathy, post-infectious cardiomyopathy, compromised cardiac function, reduced ejection fraction, heart failure, arrhythmia, and myocarditis.

Efficacy of treatment or amelioration of disease can be assessed, for example by measuring disease progression, disease remission, symptom severity, reduction in pain, quality of life, reduction of abnormal findings on cardiac testing, dose of a medication required to sustain a treatment effect, level of a disease marker or any other measurable parameter appropriate for a given disease being treated or targeted for prevention. It is well within the ability of one skilled in the art to monitor efficacy of treatment or prevention by measuring any one of such parameters, or any combination of parameters. In connection with the administration of a fusion polypeptides or pharmaceutical composition thereof, “effective against” a disease or disorder indicates that administration in a clinically appropriate manner results in a beneficial effect for at least a fraction of patients, such as an improvement of symptoms, a cure, a reduction in disease load, extension of life, improvement in quality of life, a reduction in the need for blood transfusions or other effect generally recognized as positive by medical doctors familiar with treating the particular type of disease or disorder.

A treatment or preventive effect is evident when there is a significant improvement, often statistically significant, in one or more parameters of disease status, or by a failure to worsen or to develop symptoms where they would otherwise be anticipated. As an example, a favorable change of at least 10% in a measurable parameter of disease, and preferably at least 20%, 30%, 40%, 50% or more may be indicative of effective treatment. Efficacy for a given compound or composition may also be judged using an experimental animal model for the given disease as known in the art. When using an experimental animal model, efficacy of treatment is evidenced when a statistically significant modulation in a marker or symptom is observed.

In some embodiments, DSG2 fusion polypeptides of the disclosure may be used to treat a cardiac abnormality in a subject. In some embodiments, the subject to be treated may have serum anti-DSG2 antibodies.

### Cardiomyopathies and Arrhythmias

In some embodiments, compositions of the disclosure may be used to treat arrhythmias. Arrhythmia refers to an inappropriate or abnormal pattern of electrical activity in the heart. Compositions of the disclosure may be used to treat one or more types of arrhythmias, as found in non-limiting examples of diseases such as arrhythmogenic right ventricular cardiomyopathy, sarcoidosis, post-acute sequelae of COVID-19, dilated cardiomyopathy, hypertrophic cardiomyopathy and/or restrictive cardiomyopathy. In one embodiment, patients with cardiomyopathies may demonstrate serum DSG2 autoantibodies in their serum.

In some embodiments, compositions of the disclosure may be used to treat cardiomyopathies. Cardiomyopathy refers to impairment of the structure and function of the muscular walls of the heart chambers. Compositions of the disclosure may be used to treat one or more types of cardiomyopathies, such as, but not limited to, arrhythmogenic right ventricular cardiomyopathy, sarcoidosis, post-acute sequelae of COVID-19 (PASC), dilated cardiomyopathy, hypertrophic cardiomyopathy and/or restrictive cardiomyopathy. In one embodiment, patients with cardiomyopathies may demonstrate serum DSG2 autoantibodies in their scrum.

In one embodiment, the compositions of the disclosure may be used to treat Arrhythmogenic right ventricular cardiomyopathy (ARVC). Arrhythmogenic right ventricular cardiomyopathy/dysplasia (ARVC/ARVD) is a heart muscle disorder associated with ventricular arrhythmia, heart failure, and sudden death. ARVC is a cardiac disease characterized by fulminant and recurrent arrhythmias, with the late potential development of cardiomyopathy and accompanying fibrofatty replacement of the myocardium. In addition to genetic mutations to the structural and signaling proteins of cardiomyocyte desmosomes, patient immune systems also have been implicated in ARVC. Mutations in DSG2 protein have been associated with ARVC and autoantibodies targeting DSG2 have been identified in patients with the disease. Approximately 50% of ARVC patients do not have known desmosome mutations; nevertheless, these patients can express DSG2 autoantibodies. In some embodiments, DSG2 fusion proteins may be used to treat ARVC patients who have one or more mutations in the DSG2 protein. In some aspects, the DSG2 fusion proteins may be used to treat ARVC patients with no known mutations in the DSG2 protein. In some embodiments, DSG2 fusion polypeptides of the disclosure may target DSG2 autoantibodies associated with ARVC.

Arrhythmias and/or cardiomyopathy may be clinical findings associated with an acute inflammatory process, for example caused by infections such as coxsackieviruses or coronaviruses, and such myocardial inflammation is referred to as myocarditis. In some embodiments, myocarditis may be caused by viruses, bacteria, parasites, and/or fungi. In some embodiments, compositions of the disclosure may be used to treat and/or prevent myocarditis associated with viruses. Non-limiting examples of viruses associated with myocarditis include, common cold causing adenovirus, COVID-19; hepatitis B and C; parvovirus, which causes a mild rash, usually in children (fifth disease); and/or herpes simplex virus, gastrointestinal infections causing echoviruses, mononucleosis causing Epstein-Barr virus, rubella, cytomegalovirus, and HIV.

In some embodiments, compositions of the disclosure may be used to treat and/or prevent myocarditis caused by bacteria. Non-limiting examples of bacteria associated with myocarditis include, Staphylococcus, Streptococcus, and/or Borrelia. In some embodiments, compositions of the disclosure may be used to treat and/or prevent myocarditis caused by parasites. Non-limiting examples of parasites associated with myocarditis include, Trypanosoma cruzi and Toxoplasma, including some that are transmitted by insects and can cause a condition called Chagas disease. In some embodiments, compositions of the disclosure may be used to treat and/or prevent myocarditis caused by fungi. Non-limiting examples of fungi associated with myocarditis include, Candida, Aspergillus; and other fungi, such as Histoplasma.

### Arrhythmogenic Right Ventricular Cardiomyopathy (ARVC)

Arrhythmogenic right ventricular cardiomyopathy (ARVC), also known as arrhythmogenic right ventricular dysplasia/cardiomyopathy (ARVD/C), is a complex and devastating heart disease, not uncommonly found in young individuals and athletes, which exhibits variability with respect to its clinical features. Classic ARVC clinical symptoms include palpitations, arrhythmic presyncope/syncope and sudden cardiac death due to ventricular arrhythmias, indicative of primary electrical involvement. However, ARVC patients can also exhibit clinical symptoms associated with structural disease, typically later in disease, which include myocardial remodeling consisting of thinning and dilation as well as functional deficits of the ventricles (right and/or left) and/or fibro-fatty replacement of the myocardium, indicative of primary structural involvement. The structural nature of the disease is further reinforced as ARVC is termed a “disease of the desmosome”, as human genetic studies show that approximately 40-50% of patients carry mutations in genes encoding components of the desmosomal cell-cell junction (e.g., desmoplakin (DSP), plakoglobin (JUP) plakophillin 2 (PKP2) and desmoglein-2 (DSG2)); however inheritance studies strongly suggest that abnormal genetics alone are not sufficient to cause ARVC disease.

At present there are no effective treatments for ARVC; there have been no randomized trials of treatment modalities, screening regimens, or medications specific for ARVC. As a result, treatment strategies for ARVC patients are primarily directed at symptomatic relief of the electrophysiological consequences, and are based on clinical expertise, results of retrospective registry-based studies, and studies on model systems. As a result, existing therapies for ARVC patients rely upon use of anti-arrhythmic drugs (sotalol, amniodarone and beta-blockers) that transition into more invasive options, which include implantable cardioverter defibrillators and cardiac catheter ablation if the patient becomes refractory or intolerant to anti-arrhythmic therapies. However, current therapeutic modalities have limited effectiveness in managing the disease with as many as 40% of ARVC patients dying within 10-11 years after initial diagnosis, highlighting the need for development of more effective therapies for patients with ARVC.

### COVID-19

The DSG2 fusion polypeptides or compositions containing the fusion polypeptides as described herein can be administered to treat COVID-19 or long-term effects of COVID-19.

In some embodiments, the compositions of the disclosure may be useful in treating COVID-19 and/or individuals infected with SARS-COV-2. An infected person may be symptomatic, pre-symptomatic, and asymptomatic. According to the World Health Organization (WHO), COVID-19 transmission may occur from symptomatic, pre-symptomatic, and asymptomatic people infected with SARS-COV-2. Symptomatic transmission may refer to transmission occurring before a person experiencing symptoms. Pre-symptomatic transmission may refer to transmission occurring prior to onset of symptoms of COVID-19.

COVID-19 may be associated with one or more symptoms such as, but not limited to, fever or chills, cough, shortness of breath or difficulty breathing, fatigue, muscle or body aches, headache, new loss of taste or smell, sore throat, congestion or runny nose, nausea or vomiting, diarrhea, trouble breathing, persistent pain or pressure in the chest. In some embodiments, COVID-19 infection can also be asymptomatic but still give rise to anti-DSG2 antibodies.

DSG2 fusion polypeptides may be used to treat one or more stages of COVID-19 disease. In general, adults with SARS-COV-2 infection may be grouped into the following severity of illness categories. However, the criteria for each category may overlap or vary across clinical guidelines and clinical trials, and a patient's clinical status may change over time (COVID-19 Treatment Guidelines Panel. Coronavirus Disease 2019 (COVID-19) Treatment Guidelines. National Institutes of Health. Available at www.covid19treatmentguidelines.nih.gov/. Accessed 12/11/2020, incorporated herein by reference in its entirety). In some embodiments, the compositions of the disclosure may be to treat asymptomatic or presymptomatic infection which may include individuals who test positive for SARS-COV-2 using a virologic test (i.e., a nucleic acid amplification test or an antigen test), but who have no symptoms that are consistent with COVID-19. In some embodiments, the compositions of the disclosure may be to treat mild illness which includes individuals who have any of the various signs and symptoms of COVID-19 (e.g., fever, cough, sore throat, malaise, headache, muscle pain, nausea, vomiting, diarrhea, loss of taste and smell) but who do not have shortness of breath, dyspnea, or abnormal chest imaging. In some embodiments, the compositions of the disclosure may be to treat moderate illness which may include individuals who show evidence of lower respiratory disease during clinical assessment or imaging and who have saturation of oxygen (SpO2)≥94% on room air at sea level. In some embodiments, the compositions of the disclosure may be to treat severe illness which includes individuals who have SpO2<94% on room air at sca level, a ratio of arterial partial pressure of oxygen to fraction of inspired oxygen (PaO2/FiO2)<300 mmHg, respiratory frequency >30 breaths per minute, or lung infiltrates >50%. In some embodiments, the compositions of the disclosure may be to treat critical illness which includes individuals who have respiratory failure, septic shock, and/or multiple organ dysfunction.

Methods of preventing one or more conditions associated with COVID-19 are also provided herein. In one embodiment, the compositions of the disclosure may be provided to the subject prior to the onset of symptoms but after exposure to the virus since there is an incubation period between exposure and symptom onset to the virus. The incubation period of novel coronavirus SARS-COV-2 is generally between two and fourteen days, with an average of five days (Lombardi et al., J. Hosp. Infect. 2020 doi: 10.1016/j.jhin.2020.03.003; the contents of which are herein incorporated by reference in its entirety).

Compositions of the disclosure may also be administered in combination with one or more therapeutic agents recommended for use in the treatment of COVID-19. In some embodiments, DSG2 fusion polypeptides described herein may be used in combination with one or more therapeutic agents such as, but not limited to, Remdesivir, chloroquine, hydroxychloroquine, azithromycin, Lopinavir, Ritonavir, ivermectin, interleukin inhibitors, interferons, kinase inhibitors, glucocorticosteroids, and/or SARS COV-2 monoclonal antibodies (e.g., Bamlanivimab, Casirivimab, Imdevimab).

Beginning in 2019, severe acute respiratory syndrome coronavirus 2 (SARS-COV-2) caused a pandemic infecting millions of people with coronavirus disease (referred to as COVID-19) (Wu et al., 2020 Nature 579, 265-269) which has led to over a million deaths worldwide. Patients infected with SARS-COV-2 can experience a range of clinical manifestations, ranging from no symptoms to critical illness. Emerging studies suggest that in some cases, individuals, even those who had mild versions of the disease may sometimes continue to experience symptoms long after their initial recovery. This condition has been called post-COVID-19 syndrome or “long-haul COVID-19” or “long COVID-19” or “post-acute sequelae of COVID-19 (PASC).” Current estimates are that up to 28% of patients who were infected with Covid-19 continue to have palpitations 3 months after recovering from acute COVID-19 infection (Puntmann, et al., Nature Med. 2022, 28, 2117-2123, incorporated herein by reference in its entirety). In addition, patients may also develop a reduced ejection fraction or cardiomyopathy, even after the acute infection with COVID-19 has resolved. Post-COVID-19 cardiac signs and symptoms may coexist with effects on other organ systems, but may also present alone. Patients with long-COVID may present with arrhythmia alone, cardiomyopathy alone, or both. Patients with cardiomyopathy range from those who are asymptomatic to those with fulminant heart failure, arrhythmia and/or sudden cardiac death.

The COVID-19 virus, SARS-COV-2 affects multiple organ systems, especially the lungs and heart. Elevation of cardiac biomarkers, particularly high-sensitivity troponin and/or creatine kinase MB have been commonly observed in patients in COVID-19 infection. A review of clinical analyses conducted by Bavishi et al. found that myocardial injury occurred in 20% of patients with COVID-19 infection (Prog Cardiovasc Dis. 2020 September-October; 63(5): 682-689). The plausible mechanisms of myocardial injury associated with COVID-19 include but are not limited to, 1) hyperinflammation and cytokine storm mediated through pathologic T-cells and monocytes leading to myocarditis, 2) respiratory failure and hypoxemia resulting in damage to cardiac myocytes, 3) down regulation of ACE2 expression and subsequent protective signaling pathways in cardiac myocytes, 4) hypercoagulability and development of coronary microvascular thrombosis, 5) diffuse endothelial injury, and/or, 6) inflammation and/or stress causing coronary plaque rupture or supply-demand mismatch leading to myocardial ischemia/infarction post-COVID-19 syndrome.

In some embodiments, compositions of the present disclosure may be used to treat post-COVID-19 syndrome. There have been an increasing number of reports of patients who experience persistent symptoms after recovering from acute COVID-19 and is herein referred to as “post-COVID-19 syndrome” and individuals suffering from these symptoms are commonly referred to as “long haulers.” In some embodiments, a patient may be considered to have post-COVID-19 syndrome if they suffer from one or more symptoms for up to a month, up to two months, up to three months, up to four months, up to five months, up to six months, up to a year or more.

The post-COVID-19 syndrome has also been associated with multiple organ damage, including cardiovascular damage. Imaging tests taken months after recovery from COVID-19 have shown lasting damage to the heart muscle, even in people who experienced only mild COVID-19 symptoms. Post-COVID-19 syndrome also appears to be associated with an increased risk of arrhythmia and/or myocarditis and/or cardiomyopathy.

Currently, therapeutic strategies for treating and/or managing COVID-19 and post-COVID-19 syndrome are lacking. The cardiac manifestations of COVID-19 place an overwhelmed health care system under considerable stress due to the substantial resources and potential intensive care support required for these patients. In particular, there is an urgent need for the development of treatment modalities for inhibiting inflammatory responses to reduce the incidence and mortality associated with COVID-19 and post-COVID-19 syndrome related myocardial injury. The present disclosure provides DSG2 fusion polypeptide-based compositions and methods for treating diseases such as, but not limited to COVID-19 and/or post-COVID-19 syndrome.

Compositions of the present disclosure may be used to treat one or more symptoms associated with the cardiovascular system in post-COVID-19 syndrome (i.e., post COVID-19 cardiac syndrome). One study including 100 patients recently recovered from COVID-19, cardiac magnetic resonance imaging revealed cardiac involvement in 78 patients (78%) and ongoing myocardial inflammation in 60 patients (60%), which was independent of preexisting conditions, severity and overall course of the acute illness, and the time from the original diagnosis (Puntmann et al. JAMA Cardiol. 2020; 5(11): 1265-1273). In a smaller study, 15% of athletes had evidence for myocardial abnormalities after recovery from acute COVID-19. In one embodiment, the DSG2 fusion polypeptides may be used to treat myocarditis in a subject with post-COVID-19 syndrome.

In some embodiments, the compositions described herein may be used to treat post-COVID-19 syndrome that is not associated with any cardiac indications.

In some embodiments, the compositions of the present disclosure may be used to treat subjects who show symptoms of COVID-19, for up to weeks, months, and/or years after initial diagnosis of COVID-19. In some embodiments, post-COVID-19 syndrome patients may demonstrate symptoms for and/or after 1 week, 2 weeks, 3 weeks, 4 weeks, 5 weeks, 6 weeks, 7 weeks, 8 weeks, 9 weeks, 10 weeks, 11 weeks, 12 weeks, 1 month, 2 months, 3 months, 4 months, 5 months, 6 months, 7 months, 8 months, 9 months, 10 months, 11 months, 12 months, 1 year, 2 years, 3 years, 4 years, 5 years or more after initial diagnosis of COVID-19. Diagnosis of COVID-19 may be established using methods known in the art (for e.g., reverse transcription polymerase chain reaction and/or antibody tests). In some embodiments, subjects affected by post-COVID-19 syndrome may be treated with the compositions of the disclosure.

In some embodiments, compositions of the disclosure may be used to treat COVID-19 patients who develop compromised cardiac function, most notably a reduced ejection fraction, with or without overt symptoms of heart failure. In some embodiments, the compositions of the disclosure may be used to treat arrhythmia.

Compositions of the present disclosure may ameliorate one or more symptoms associated with post-COVID-19 syndrome. In some embodiments, the symptoms of post-COVID-19 syndrome may be the same as acute COVID-19. In some aspects, the symptoms associated with post-COVID-19 syndrome may be shortness of breath, fatigue, edema, orthopnea, limitations to exertion, impaired cognitive abilities, palpitations, dizziness, syncope, lightheadedness, heart failure, and/or arrhythmia.

In some embodiments, compositions of the disclosure may be used to treat post COVID-19 syndrome with symptoms that overlap with the post-intensive care syndrome that has also been described in patients without COVID-19.

In some embodiments, the compositions of the disclosure may be used to treat subjects with post-COVID-19 syndrome who may have one or more long-term complications associated with the cardiovascular system (e.g., inflammation of the heart muscle), respiratory system (lung function abnormalities), renal systems (acute kidney injury), dermatologic (rash, hair loss), neurological complications (smell and taste problems, sleep issues, difficulty with concentration, memory problems), and/or psychiatric problems (depression, anxiety, changes in mood).

In some embodiments, compositions of the disclosure may be used to treat post-COVID-19 syndrome subjects who may have one, two or more associated co-morbidities. Non-limiting examples of co-morbidities include, but are not limited to, hypertension, thyroid disease, immune disorders, COPD (chronic obstructive pulmonary disease), high blood pressure, obesity, mental health conditions, and diabetes.

### V. Definitions

Domain: As used herein when referring to polypeptides the term “domain” refers to a motif of a polypeptide having one or more identifiable structural or functional characteristics or properties (e.g., binding capacity, serving as a site for protein-protein interactions).

Expression vector: The term “expression vector” as used herein refers to a vector containing a nucleic acid sequence coding for at least part of a gene product capable of being transcribed. Expression vectors can contain a variety of control sequences, which refer to nucleic acid sequences necessary for the transcription and possibly translation of an operatively linked coding sequence in a particular host organism. In addition to control sequences that govern transcription and translation, vectors and expression vectors may contain nucleic acid sequences that serve other functions as well. The term also includes a recombinant plasmid or virus that comprises a polynucleotide to be delivered into a host cell, either in vitro or in vivo. In some embodiments, the host cell is a transient cell line or a stable cell line. In some embodiments, it is selected from the group consisting of CHO, HEK293 and NS0 cells.

Features: “Features” when referring to polypeptides are defined as distinct amino acid sequence-based components of a molecule. Features of the polypeptides encoded by the polynucleotides described herein include local conformational shape, folds, loops, half-loops, domains, half-domains, sites, termini, or any combination thereof.

Fusion protein: As used herein, the term “fusion protein” or chimeric protein refers to protein or polypeptide comprising two or more sequences of amino acids or active fragments thereof that are not naturally present in the same polypeptide. In some embodiments, two or more separate polypeptides are operably covalently linked, e.g., chemically linked, or fused together by peptide bonds. Recombinant fusion polypeptides are created artificially by recombinant DNA technology.

Half-domain: As used herein when referring to polypeptides the term “half-domain” means a portion of an identified domain having at least half the number of amino acids residing on the domain from which it is derived. It is understood that domains may not always contain an even number of amino acid residues. Therefore, in those cases where a domain contains or is identified to comprise an odd number of amino acids, a half-domain of the odd-numbered domain will comprise the whole number portion or next whole number portion of the domain (number of amino acids of the domain/2+/−0.5 amino acids). For example, a domain identified as a 7 amino acid domain could produce half-domains of 3 amino acids or 4 amino acids (7/2=3.5±1−0.5 being 3 or 4). It is also understood that sub-domains may be identified within domains or half-domains, these subdomains possessing less than all of the structural or functional properties identified in the domains or half domains from which they were derived. It is also understood that the amino acids that comprise any of the domain types herein need not be contiguous along the backbone of the polypeptide (i.e., nonadjacent amino acids may fold structurally to produce a domain, half-domain or subdomain).

Immune response: As used herein, the term “immune response” refers to conditions associated with inflammation, trauma, immune disorders, or infectious or genetic disease. These conditions can be characterized by expression of various factors, e.g., antibodies, immune cells, cytokines, chemokines, and other signaling molecules, which may affect cellular and systemic defense systems.

Linker: As used herein, “linker” refers to a functional group (e.g., a chemical or polypeptide) in which a covalent bond joins two or more polypeptides. As used herein, a “peptide linker” is two or more amino acids used to bind two proteins to each other.

Modulation: As used herein, the term “modulation” is recognized in the art and refers to up regulation (i.e., activation or stimulation), down regulation (i.e., inhibition or suppression) of a response, or the two in combination or apart.

Polynucleotide: The term “polynucleotide” as used herein refers to a sequence of nucleotides connected by phosphodiester linkages. Polynucleotides are presented herein in the direction from the 5′ to the 3′ direction. A polynucleotide can be a deoxyribonucleic acid (DNA) molecule or ribonucleic acid (RNA) molecule. Where a polynucleotide is a DNA molecule, that molecule can be a gene or a cDNA molecule. Nucleotide bases are indicated herein by a single letter code: adenine (A), guanine (G), thymine (T), cytosine (C), inosine (I) and uracil (U). A polynucleotide can be prepared using standard techniques well known to one of skill in the art.

Polypeptides: In some embodiments, the compositions of the present disclosure are polypeptides or proteins or variants thereof. According to the present disclosure, any amino acid-based molecule (natural or non-natural) may be termed a “polypeptide” and this term embraces “peptides,” “peptidomimetics,” and “proteins.” As used herein, “polypeptide” means a polymer of amino acid residues (natural or unnatural) linked together most often by peptide bonds. The term, as used herein, refers to proteins, polypeptides, and peptides of any size, structure, or function. A “peptidomimetic” or “polypeptide mimetic” is a polypeptide in which the molecule contains structural elements that are not found in natural polypeptides (i.e., polypeptides comprised of only the 20 proteinogenic amino acids). In some embodiments, peptidomimetics are capable of recapitulating or mimicking the biological action(s) of a natural peptide.

Polypeptide variant: The term “polypeptide variant” refers to molecules which differ in their amino acid sequence from a native or reference sequence. The amino acid sequence variants may possess substitutions, deletions, and/or insertions at certain positions within the amino acid sequence, as compared to a native or reference sequence. Ordinarily, variants will possess at least about 50% identity (homology) to a native or reference sequence, and preferably, they will be at least about 80%, more preferably at least about 90% identical (homologous) to a native or reference sequence.

Recombinant: The term “recombinant” as used herein refers to a genetic entity distinct from that generally found in nature. As applied to a polynucleotide or gene, this means that the polynucleotide is the product of various combinations of cloning, restriction and/or ligation steps, and other procedures that result in the production of a construct that is distinct from a polynucleotide found in nature.

Sample: As used herein, the term “sample” refers to an aliquot or portion taken from a source and/or provided for analysis or processing. In some embodiments, a sample is from a biological source such as a tissue, cell or component part (e.g., a body fluid, including but not limited to blood, mucus, lymphatic fluid, synovial fluid, cerebrospinal fluid, saliva, amniotic fluid, amniotic cord blood, urine, vaginal fluid and semen). In some embodiments, a sample may be or include a homogenate, lysate or extract prepared from a whole organism or a subset of its tissues, cells or component parts, or a fraction or portion thereof, including but not limited to, for example, plasma, serum, spinal fluid, lymph fluid, the external sections of the skin, respiratory, intestinal, and genitourinary tracts, tears, saliva, milk, blood cells, tumors, or organs. In some embodiments, a sample is or includes a medium, such as a nutrient broth or gel, which may contain cellular components, such as proteins. In some embodiments, a “primary” sample is an aliquot of the source. In some embodiments, a primary sample is subjected to one or more processing (e.g., separation, purification, etc.) steps to prepare a sample for analysis or other use.

Sequence identity: The term “sequence identity” indicates the percentage of identical nucleotides or amino acids indicated by a sequence alignment. For example, two peptides, each having 20 amino acid residues which are identical in amino acid sequence except for differences at two positions will have 18/20, or 90% sequence identity.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Terminus: As used herein the terms “termini” or “terminus” when referring to polypeptides refers to an extremity of a peptide or polypeptide. Such extremity is not limited only to the first or final site of the peptide or polypeptide but may include additional amino acids in the terminal regions. The polypeptide-based molecules described herein may be characterized as having both an N-terminus (terminated by an amino acid with a free amino group (NH2)) and a C-terminus (terminated by an amino acid with a free carboxyl group (COOH)). Proteins described herein are in some cases made up of multiple polypeptide chains brought together by disulfide bonds or by non-covalent forces (multimers, oligomers). These sorts of proteins will have multiple N- and C-termini. Alternatively, the termini of the polypeptides may be modified such that they begin or end, as the case may be, with a non-polypeptide-based moiety such as an organic conjugate.

Therapeutically effective amount: As used herein, the term “therapeutically effective amount” means an amount of an agent to be delivered that is sufficient, when administered to a subject suffering from or susceptible to a disease, disorder, and/or condition, to treat, improve symptoms of, diagnose, prevent, and/or delay the onset of the disease, disorder, and/or condition.

Treating: As used herein, the term “treating” refers to partially or completely alleviating, ameliorating, improving, relieving, delaying onset of, inhibiting progression of, reducing severity of, and/or reducing incidence of one or more symptoms or features of a particular disease, disorder, and/or condition. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition.

Treatment: As used herein the terms “treat,” “treatment,” and the like, refer to relief from or alleviation of pathological processes. In the context of the present disclosure, it relates to any of the other conditions recited herein below, the terms “treat,” “treatment,” and the like mean to relieve or alleviate at least one symptom associated with such condition, or to slow or reverse the progression or anticipated progression of such condition.

Treatment dose: As used herein, “treatment dose” refers to one or more doses of a therapeutic agent administered in the course of addressing or alleviating a therapeutic indication. Treatment doses may be adjusted to maintain a desired concentration or level of activity of a therapeutic agent in a body fluid or biological system.

### VI. Equivalents and Scope

While various embodiments of the disclosure have been particularly shown and described in the present disclosure, it will be understood by those skilled in the art that various changes in form and details may be made without departing from the spirit and scope of the embodiments disclosed herein and set forth in the appended claims.

Those skilled in the art will recognize or be able to ascertain using no more than routine experimentation, many equivalents to the specific embodiments described herein. The scope of the present disclosure is not intended to be limited to the above description, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of a group is present in, employed in, or otherwise relevant to a given product or process. The disclosure includes embodiments in which more than one, or all group members are present in, employed in, or otherwise relevant to a given product or process.

It is also noted that the term “comprising” is intended to be open and permits but does not require the inclusion of additional elements or steps. When the term “comprising” is used herein, the terms “consisting of” and “or including” are thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the disclosure, to the tenth of the unit of the lower limit of the range, unless the context clearly dictates otherwise.

In addition, it is to be understood that any particular embodiment of the present disclosure that falls within the prior art may be explicitly excluded from any one or more of the claims. Since such embodiments are deemed to be known to those of ordinary skill in the art, they may be excluded even if the exclusion is not set forth explicitly herein. Any particular embodiments of compositions disclosed herein can be excluded from any one or more claims, for any reason, whether or not related to the existence of prior art.

All cited sources, for example, references, publications, databases, database entries, and art cited herein, are incorporated into this application by reference, even if not expressly stated in the citation. In case of conflicting statements of a cited source and the instant application, the statement in the instant application shall control.

Section and table headings are not intended to be limiting.

### EXAMPLES

### Example 1. DSG2 Fusion Polypeptides and Interaction with Anti-DSG2 Antibody

The candidates for DSG2 fusion polypeptides include the extracellular domain (ECD) portion of DSG2 fused to IgG Fc or variants thereof. Linking of ECD to Fc portion of immunoglobulins was considered to be beneficial to DSG2 fusion polypeptides as it could provide increased protein stability, enhance PK properties and efficient purification. Table 4 provides the DSG2 fusion polypeptides that were obtained or prepared.

It was noted that the whole preparation of the DSG2 fusion polypeptides were highly heterogenous as seen by one-step affinity purification. Early assessment of production suggested high aggregation. As shown in Table 5, dynamic light scattering (DLS) shows that all candidates showed average polymer distribution index (PDI) of >0.25 indicating that the sample may contain multiple size particles, either due to more aggregation or precipitation. 6 candidates were compared to the reference sample. All six candidates showed the peak 1 mode diameter of about 4- to 8-fold higher in size. IgG4 containing fusion polypeptides showed the peak 1 mode diameter of about 2-fold higher in size as compared to IgG1 containing fusion polypeptides.

TABLE 5
DLS data interpretation of DSG2
fusion polypeptides

| | DSG2 | | | |
|---|---|---|---|---|
| | fusion | Peak 1 | | Peak |
| | polypeptide | mode | | 1 |
| DSG2 fusion | number | diameter | | mass |
| polypeptide name | (DSG2FP#) | (nm) | PDI | % |
| rhDesmoglein-2/Fc | DSG2FP#1 | 18.13 | 0.33 | ~97% |
| Chimera | | | | |
| DSG2-(IgG1)Fc | DSG2FP#2 | 40.04 | 0.32 | ~96% |
| DSG2-GGGGS-(IgG1)Fc | DSG2FP#3 | 43.23 | 0.30 | ~91% |
| DSG2-EAAAK-(IgG1)Fc | DSG2FP#4 | 44.87 | 0.32 | ~96% |
| (IgG1)Fc-DSG2 | DSG2FP#5 | 76.72 | 0.31 | ~99% |
| DSG2-(IgG4)Fc | DSG2FP#6 | 98.03 | 0.32 | ~99% |
| DSG2-GGGGS-(IgG4)Fc | DSG2FP#7 | 86.23 | 0.30 | ~97% |

Table 6 shows melting temperature Tm values for the candidate DSG2 fusion polypeptides. The presence of three Tm values suggested that the candidate DSG2 fusion polypeptides may contain aggregates prior to analysis. As the temperature increased, the aggregates began to move, unfold and spread apart. The differential graph in the 4 samples containing the third Tm value showed a transition after the melting point. The Tm values provided during analysis demonstrated similar values among all the DSG2 fusion polypeptides. This may suggest the thermal stability of the constructs are similar.

TABLE 6
Tm of DSG2 fusion polypeptides

| | DSG2 | | | |
|---|---|---|---|---|
| | fusion | | | |
| | polypeptide | | | |
| DSG2 fusion | number | Tm1 | Tm2 | Tm3 |
| polypeptides | (DSG2FP#) | (° C.) | (° C.) | (° C.) |
| rhDesmoglein-2/Fc | DSG2FP#1 | 49.9 | 64.1 | 77.5 |
| Chimera | | | | |
| DSG2-(IgG1)Fc | DSG2FP#2 | 52.0 | 61.9 | 81.9 |
| DSG2-GGGGS-(IgG1)Fc | DSG2FP#3 | 51.3 | 62.8 | 82.1 |
| DSG2-EAAAK-(IgG1)Fc | DSG2FP#4 | 50.7 | 63.1 | 81.5 |
| (IgG1)Fc-DSG2 | DSG2FP#5 | 65.0 | — | — |
| DSG2-(IgG4)Fc | DSG2FP#6 | 51.1 | 60.0 | — |
| DSG2-GGGGS-(IgG4)Fc | DSG2FP#7 | 53.4 | 60.0 | — |

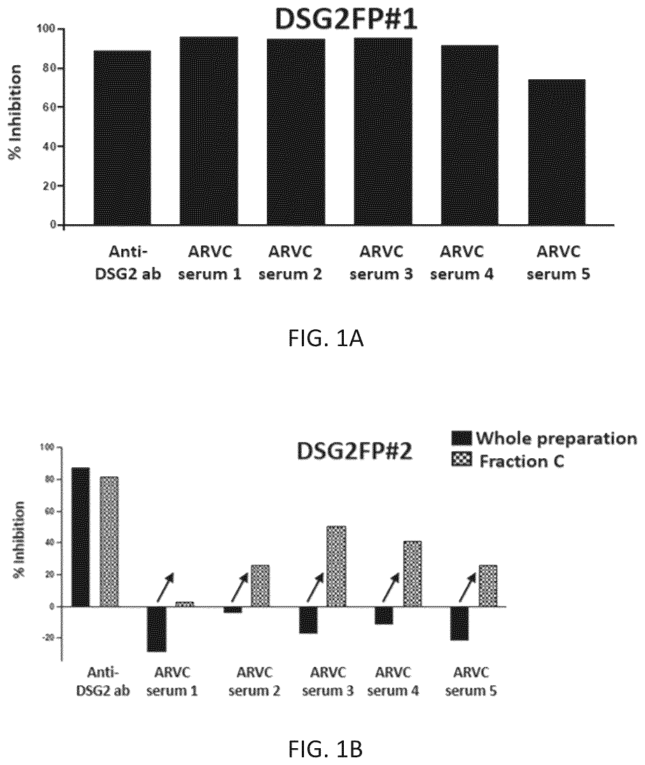

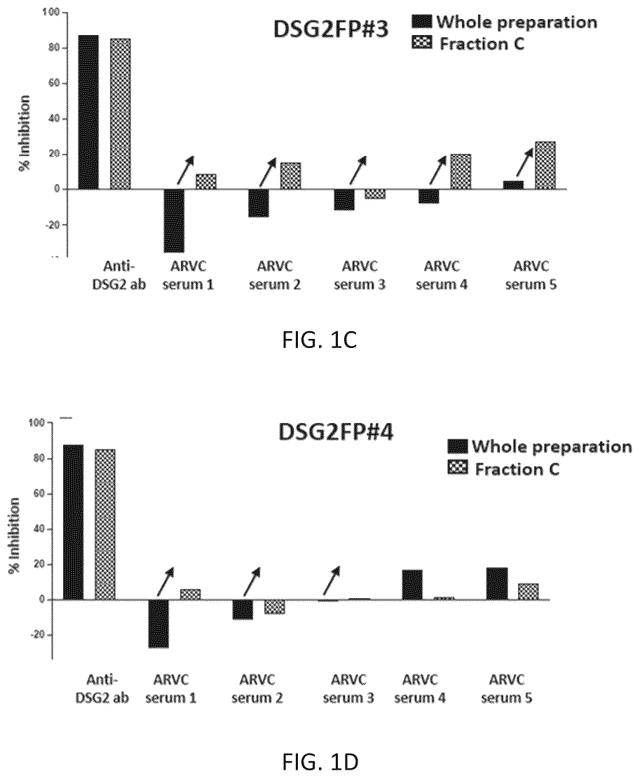

The DSG2 fusion polypeptides were tested for the ability to block anti-DSG2 antibody by an anti-DSG2 antibody meso scale discovery (MSD) assay. DSG2FP #2, DSG2FP #3, DSG2FP #4, DSG2FP #5, DSG2FP #6, DSG2FP #7 fusion polypeptides tested were able to bind to and block anti-DSG2 antibodies. DSG2 fusion polypeptide DSG2FP #1 was able to bind and block anti-DSG2 antibody as well as serum obtained from ARVC patients (herein also referred to as ARVC serum) (See FIG. 1A). The DSG2 fusion polypeptides were subjected to size exclusion chromatography and different elution fractions were examined. Fraction “C” showed a predicted MW of ˜350 kDa and was used in additional experiments. As shown in Table 7 and FIG. 1B, FIG. 1C, and FIG. 1D, enriching for fraction C improved the ability of the DSG2 fusion polypeptides to block ARVC patient serum. This effect is more pronounced for DSG2FP #2 and DSG2FP #3 than observed for DSG2FP #4.

TABLE 7
Effect of fraction C of DSG2 fusion polypeptides on ARVC serum

| | DSG2FP#1 (% | DSG2FP#3 (% | DSG2FP#4 (% | DSG2FP#2 (% |
|---|---|---|---|---|
| Sample | inhibition) | inhibition) | inhibition) | inhibition) |
| Anti-DSG2 antibody | 88.4 | 85.5 | 85.5 | 81.6 |
| (1000 ng/mL) control | | | | |
| Anti-DSG2 antibody | 63.8 | 61.2 | 61.2 | 54.5 |
| (200 ng/mL) control | | | | |
| no antibody control | 4.8 | 0.7 | 0.7 | −19.1 |
| ARVC serum 1 | 96.7 | 9.2 | 6.0 | 3.0 |
| ARVC serum 2 | 82.9 | 27.6 | 9.2 | 33.5 |
| ARVC serum 3 | 95.8 | 15.5 | −8.0 | 26.0 |
| ARVC serum 4 | 95.7 | −5.6 | 0.8 | 51.1 |
| ARVC serum 5 | 93.1 | 20.6 | 2.0 | 41.8 |

### Example 2. Effect of Anti-DSG2 Antibodies and DSG2 Fusion Polypeptides on Cardiac Function

To determine the effect of anti-DSG2 antibodies in cardiomyocytes, the cardiac in vitro proarrhythmia (CiPA) assay was performed (Sager et al. Am Heart J 2014; 167:292-300; the contents of which are herein incorporated by reference in its entirety). CiPA is envisioned to consist of four components: (1) an evaluation of the effects of test agents on cardiac ion channel assays; (2) in silico modeling of the cardiac action potential based on the ion channel results to integrate the data with cardiac function; (3) experimental measurement of test agent effects in human ventricular myocytes to confirm the modeling result; and (4) scoring the results. The multielectrode array (MEA) assay is a component of CiPA which uses human induced pluripotent stem cell (iPSC)-derived cardiomyocytes to evaluate two-dimensional excitation propagation by recording spontaneous ECG-like field potentials. The sodium spike quantified below is a measurement of the specific sodium channel current in the cardiomyocyte and is also described in Blinova K, et al. Cell Rep. 2018 Sep. 25; 24(13):3582-3592 (the contents of which are herein incorporated by reference in its entirety). CiPA is considered a clinically validated cardiomyocyte-based assay by the FDA and the EMA. An arrhythmia signal in the assay is considered evidence that a certain agent, e.g. anti-DSG2 antibody, has an arrhythmic potential in humans. The parameters evaluated include measuring a change in the spike in the component of electrical signal propagation (e.g. sodium spike) and cell index—a function of cell impedance and cell number. Changes in sodium spike may be indicative of arrhythmia and a change in cell index (CI) may be indicative of changes to cell connectivity.

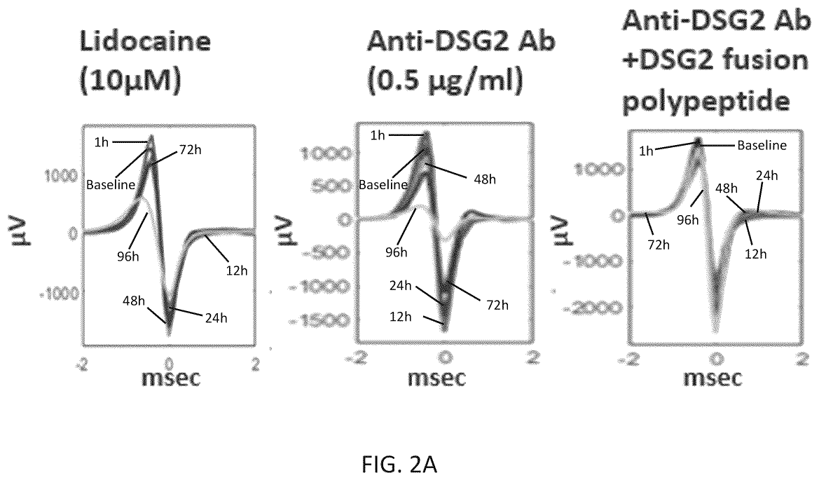

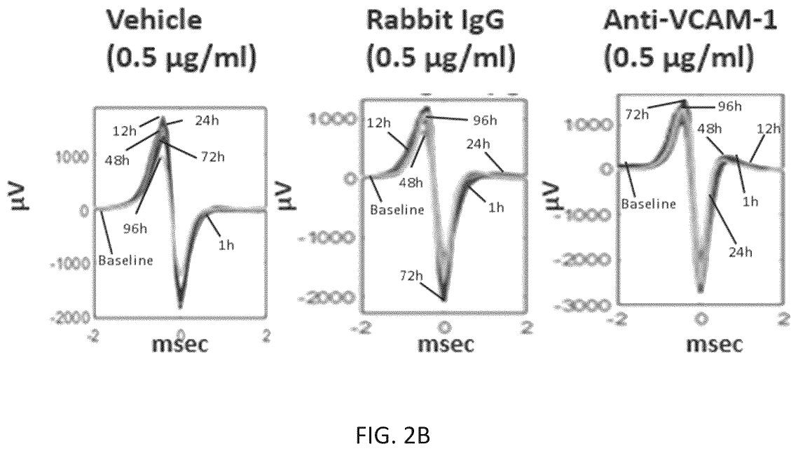

Commercially available anti-DSG2 antibodies were tested in the cardiomyocyte CiPA assay. Anti-DSG2 antibodies were found to dampen the cardiomyocyte sodium spike and this effect was time and dose-dependent. The voltage (μV) is measured at time intervals of 0, 1 hour, 12 hours, 24 hours, 48 hours, 72 hours, and 96 hours. As shown in FIG. 2, the effect of anti-DSG2 antibodies (0.5 μg/mL) on sodium spike was comparable to the effect of 10 μM lidocaine (positive control). The effect of anti-DSG2 antibodies on cardiomyocyte sodium spike was rescued by 1.1 μg/mL of DSG2 fusion polypeptide, DSG2FP #1. As expected, the negative controls, vehicle, rabbit IgG and anti-VCAM-1 antibodies at 0.5 g/mL did not cause any changes in the sodium spike over time.

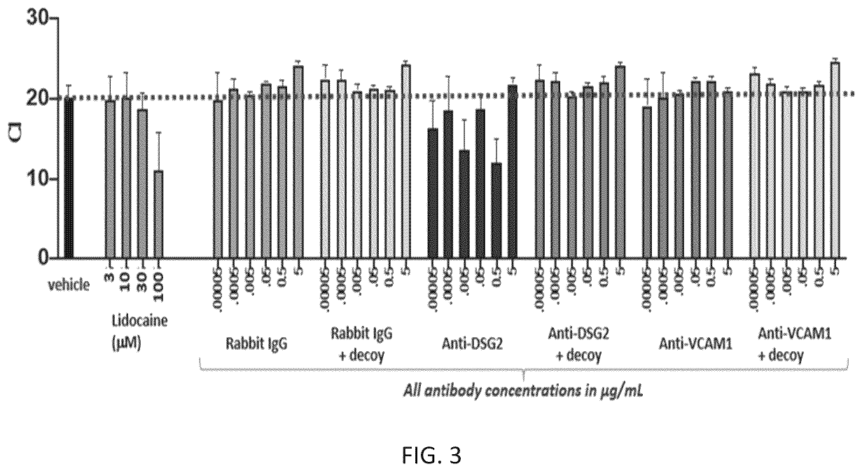

Varying doses of anti-DSG2 antibodies affected cell index. Cell index is inversely related the cell layer's conductivity to electrical current. Increased conductivity across the cell layer, may indicate that there are more gaps in the cell layer, implying that the cells are less well-adhered to each other. Thus, increased conductivity across the cell layer, may result in lower cell index. No dose response was observed. The concentration of anti-DSG2 antibodies varied from 0.00005 μg/mL to 5 μg/mL. Treatment with high doses of lidocaine (positive control) resulted in marked reduction in cell index and likely cell death. The concentration of lidocaine varied from 3 μM to 100 μM. Notably, none of the negative controls (vehicle, anti-VCAM-1 antibody or rabbit IgG) showed a reduction in cell index. The concentration of anti-DSG2 antibodies varied from 0.00005 μg/mL to 5 μg/mL. Effects of anti-DSG2 antibodies on cell index were reversed by addition of DSG2 fusion polypeptide, DSG2FP #1 (See FIG. 3 and Table 8).

TABLE 8
Varying doses of anti-DSG2 antibodies affected cell
index in iPSC-derived cardiomyocyte MEA assay

| | Cell Index |
|---|---|
| Condition | (+/− SEM) |
| Vehicle | 20.1 (1.5) |
| Lidocaine (3 μM) | 19.9 (2.84) |
| Lidocaine (10 μM) | 20.2 (3.1) |
| Lidocaine (30 μM) | 18.7 (2.0) |
| Lidocaine (100 μM) | 11.0 (4.8) |
| Rabbit IgG (0.00005 μg/mL) | 19.9 (3.5) |
| Rabbit IgG (0.0005 μg/mL) | 21.2 (1.2) |
| Rabbit IgG (0.005 μg/mL) | 20.5 (0.3) |
| Rabbit IgG (0.05 μg/mL) | 22.0 (0.2) |
| Rabbit IgG (0.5 μg/mL) | 21.5 (0.8) |
| Rabbit IgG (5 μg/mL) | 24.2 (0.5) |
| Rabbit IgG (0.00005 μg/mL) + DSG2FP#1 | 22.5 (1.7) |
| Rabbit IgG (0.0005 μg/mL) + DSG2FP#1 | 22.4 (1.3) |
| Rabbit IgG (0.005 μg/mL) + DSG2FP#1 | 21.0 (0.8) |
| Rabbit IgG (0.05 μg/mL) + DSG2FP#1 | 21.3 (0.4) |
| Rabbit IgG (0.5 μg/mL) + DSG2FP#1 | 21.1 (0.4) |
| Rabbit IgG (5 μg/mL) + DSG2FP#1 | 24.2 (0.5) |
| Anti-DSG2 antibody (0.00005 μg/mL) | 16.3 (3.5) |
| Anti-DSG2 antibody (0.0005 μg/mL) | 18.6 (4.2) |
| Anti-DSG2 antibody (0.005 μg/mL) | 13.6 (3.7) |
| Anti-DSG2 antibody (0.05 μg/mL) | 18.7 (1.8) |
| Anti-DSG2 antibody (0.5 μg/mL) | 12.0 (2.9) |
| Anti-DSG2 antibody (5 μg/mL) | 21.8 (0.9) |
| Anti-DSG2 antibody (0.00005 μg/mL) + DSG2FP#1 | 22.4 (1.9) |
| Anti-DSG2 antibody (0.0005 μg/mL) + DSG2FP#1 | 22.4 (1.1) |
| Anti-DSG2 antibody (0.005 μg/mL) + DSGFP#1 | 20.4 (0.5) |
| Anti-DSG2 antibody (0.05 μg/mL) + DSGFP#1 | 21.6 (0.4) |
| Anti-DSG2 antibody (0.5 μg/mL) + DSGFP#1 | 22.0 (0.8) |
| Anti-DSG2 antibody (5 μg/mL) + DSGFP#1 | 24.1 (0.5) |
| Anti-VCAM1 antibody (0.00005 μg/mL) | 19.1 (3.4) |
| Anti-VCAM1 antibody (0.0005 μg/mL) | 20.1 (3.1) |
| Anti-VCAM1 antibody (0.005 μg/mL) | 20.7 (0.5) |
| Anti-VCAM1 antibody (0.05 μg/mL) | 22.3 (0.4) |
| Anti-VCAM1 antibody (0.5 μg/mL) | 22.3 (0.5) |
| Anti-VCAM1 antibody (5 μg/mL) | 21.0 (0.4) |
| Anti-VCAM1 antibody (0.00005 μg/mL) + DSG2FP#1 | 23.2 (0.7) |
| Anti-VCAM1 antibody (0.0005 μg/mL) + DSG2FP#1 | 22.0 (0.5) |
| Anti-VCAM1 antibody (0.005 μg/mL) + DSG2FP#1 | 21.0 (0.5) |
| Anti-VCAM1 antibody (0.05 μg/mL) + DSG2FP#1 | 21.0 (0.4) |
| Anti-VCAM1 antibody (0.5 μg/mL) + DSG2FP#1 | 21.8 (0.5) |
| Anti-VCAM1 antibody (5 μg/mL) + DSG2FP#1 | 24.6 (0.5) |

### Example 3. DSG2 Antibody Levels in ARVC Patients

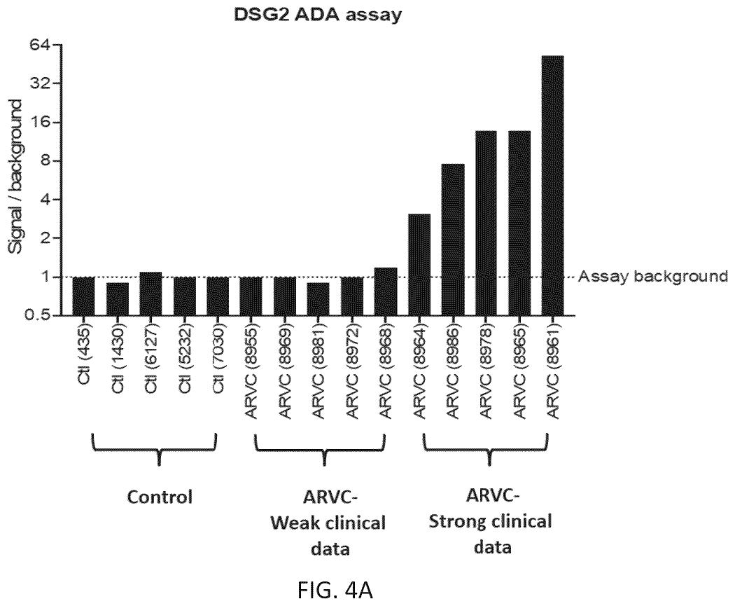

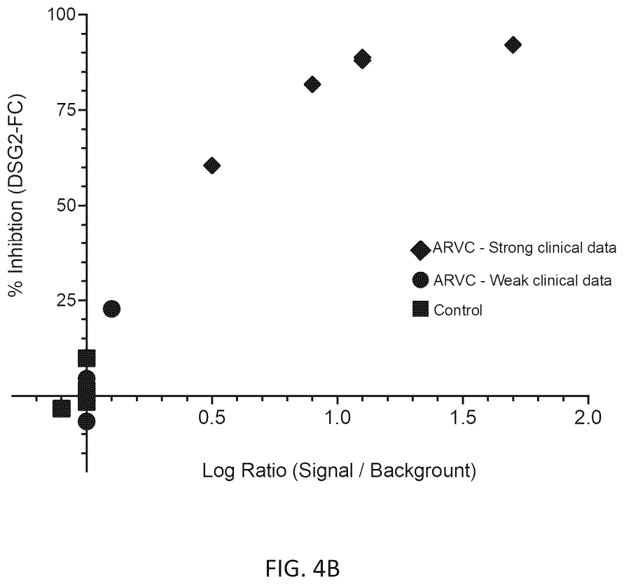

ARVC diagnosis is based on complex tool known as Task Force Criteria, which can lead to varying levels of conviction about ARVC diagnosis. The utility of DSG2 antibodies as an indicator of ARVC disease state and severity was explored. Approximately 50 cc serum was collected from diagnosed ARVC patients. A total of 10 ARVC patients and 5 healthy controls were used for the analysis. ARVC serum samples were separated into more robust diagnosis and less robust diagnosis, based on available data in the patient medical records and anti-DSG2 antibody levels were compared using an anti-DSG2 antibody meso scale discovery (MSD)-based assay. The assay results showed that anti-DSG2 antibody signal strength in the assay correlated well with the robustness of the diagnostic clinical data. ARVC serum samples obtained from patients with strong clinical data showed higher levels of anti-DSG2 antibodies compared to the other two group (FIG. 4A and FIG. 4B). The assay signal from the patient serum samples with strong clinical data could also be blocked by adding a competing DSG2 polypeptide DSG2-Fc (DSG2FP #1).

### Example 4. Blocking of Anti-DSG2 Antibodies Present in ARVC Patient Sera from Binding to the DSG2 Extracellular Domain by DSG2 Fusion Polypeptides with Different Affinity Tags

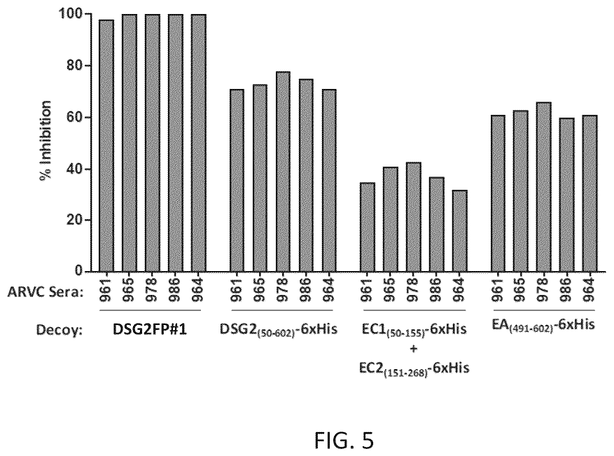

DSG2 fusion polypeptides were investigated for the ability to block the binding of anti-DSG2 antibodies in sera of ARVC patients to the DSG2 extracellular domain in an electrochemical immunoassay. The fusion polypeptides were added to a final concentration of 5 μg/mL and patient sera were used at a final concentration of 10% (v/v). The results are shown in the series of bar charts in FIG. 5, where the numbers 961, 965, 978, 986 and 964 indicate ARVC patient serum samples.

In FIG. 5, the first data series from the left is for DSG2FP #1 (described above as amino acids 49-608 of SEQ ID NO:30; Linker (IEGRMD (SEQ ID NO: 28)); amino acids 100-330 of IgG1 (SEQ ID NO: 4). The second data series from the left is for DSG2(50-602)-6×His, which is a fusion polypeptide of DSG2 amino acids 50-602 of SEQ ID NO: 1 (complete extracellular domain) to a hexahistidine peptide tag. The third data series from the left is for a combination of fusion polypeptides which includes EC1(50-155)-6×His, a fusion of DSG2 amino acids 50-155 of SEQ ID NO: 1 (EC1) to a hexahistidine peptide tag and EC2(151-268)-6×His, a fusion of DSG2 amino acids 151-268 of SEQ ID NO: 1 (EC2) to a hexahistidine peptide tag. The fourth data series from the left is for EA(491-602)-6×His, a fusion of DSG2 amino acids 491-602 of SEQ ID NO: 1 (EA) to a hexahistidine peptide tag. The inhibition results of FIG. 5, where 100% inhibition indicates complete blocking, show that the best blocking activity is provided by DSG2FP #1. Two additional assays indicate at least 60% blocking activity by fusion polypeptides which include hexahistidine tags instead of the IgG1 Fc region. It is therefore apparent that blocking of the binding of anti-DSG2 antibodies in sera of ARVC patients to the DSG2 extracellular domain can be accomplished with DSG2 fusion polypeptides having affinity tags other than the Fc region of an IgG. It is likely that other affinity tags may be determined to be useful in alternative embodiments of DSG2 polypeptides, with examples including, but not limited to CBP, MBP, streptavidin and GST, for example. In addition, the fusion polypeptides having portions of the extracellular domain (ECD), including the EA region, demonstrate blocking of the binding of anti-DSG2 antibodies in sera of ARVC patients to the DSG2 extracellular domain. These findings provide a basis for predicting that fusion polypeptides comprising the DSG2 extracellular domain or a portion thereof, in combination with different affinity tags will be appropriate therapeutic agents for use in treatment of disorders arising from anti-DSG2 autoantibodies, including various cardiac arrhythmias which function by blocking the interaction of the anti-DSG2 antibodies with the extracellular domain of DSG2.

### Example 5. Blocking of Anti-DSG2 Antibodies Binding to the DSG2 Extracellular Domain by DSG2 Fusion Polypeptides with an IgG4 Affinity Tag

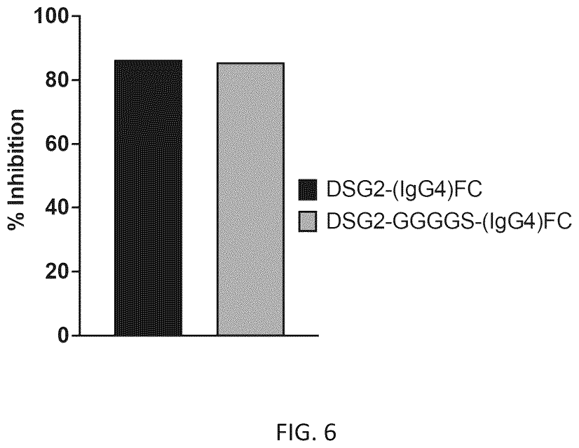

Blocking of binding of anti-DSG2 antibodies to the DSG2 extracellular domain by two recombinant DSG2 fusion polypeptides was assessed in an electrochemical immunoassay. The fusion polypeptides were added to a final concentration of 5 mg/mL. Anti-DSG2 antibodies were used at final concentration of 500 ng/mL. The results are presented in FIG. 6. Complete blocking in the assay is 100% inhibition. DSG2 recombinant proteins: The black bar is for DSG2-(IgG4)FC (DSG2FP #6), a fusion protein of the DSG2 extracellular domain represented by amino acids 50-609 of SEQ ID NO: 1 to IgG4 Fc domain amino acids 99-332 of SEQ ID NO: 11. The grey bar is for DSG2-GGGGS-(IgG4)FC (DSG2FP #7), a fusion protein of the full-length of the DSG2 extracellular domain represented by amino acids 50-609 of SEQ ID NO: 1 to a GGGGS peptide linker (SEQ ID NO: 12) and IgG4 Fc domain amino acids 99-332 of SEQ ID NO: 11.

Both fusion polypeptides show similar activity at greater than 80% inhibition. This indicates that usage of the human IgG4 Fc region as an affinity tag does not impair the anti-DSG2 antibody blocking function of the fusion polypeptides and the presence of a linker in this case does not produce a significant effect on the inhibition activity. These findings provide a basis for predicting that fusion polypeptides comprising the DSG2 extracellular domain or a portion thereof, in combination with different affinity tags, including Fc regions from other immunoglobulins will be appropriate therapeutic agents for use in treatment of disorders arising from anti-DSG2 autoantibodies, including various cardiac arrhythmias.

1. An isolated polypeptide comprising a desmoglein-2 (DSG2) fusion polypeptide, wherein the DSG2 fusion polypeptide comprises:
a. a whole or a portion of a DSG2 protein, wherein the DSG2 protein is a Homo sapiens DSG2 protein, a Mus musculus DSG2 protein, a Rattus norvegicus DSG2 protein, a Macaca mulatta DSG2 protein, a Canis lupus familiaris DSG2 protein, or a Danio rerio DSG2 protein; and
b. a whole or a portion of an immunoglobulin protein, wherein the immunoglobulin protein is a human or a canine immunoglobulin protein.

2. The isolated polypeptide of claim 1, wherein the DSG2 protein is a Homo sapiens DSG2 protein (SEQ ID NO. 1).

3. The isolated polypeptide of claim 1, wherein the DSG2 protein is a Mus musculus DSG2 protein (SEQ ID NO. 14).

4. The isolated polypeptide of claim 1, wherein the DSG2 protein is a Rattus norvegicus DSG2 protein (SEQ ID NO. 15).

5. The isolated polypeptide of claim 1, wherein the DSG2 protein is a Macaca mulatta DSG2 protein (SEQ ID NO. 16).

6. The isolated polypeptide of claim 1, wherein the DSG2 protein is a Canis lupus familiaris DSG2 protein (SEQ ID NO. 17).

7. The isolated polypeptide of claim 1, wherein the DSG2 protein is a Danio rerio DSG2 protein (SEQ ID NO. 18).

8. The isolated polypeptide of claim 1, wherein the DSG2 fusion polypeptide comprises a portion of the DSG2 protein.

9. The isolated polypeptide of claim 8, wherein the portion of the DSG2 protein is a whole or a portion of an extracellular region of the DSG2 protein.

10. The isolated polypeptide of claim 1, wherein the portion of DSG2 protein is the whole extracellular region of DSG2 protein.

11. The isolated polypeptide of claim 1, wherein the DSG2 fusion polypeptide comprises a portion of an immunoglobulin protein.

12. The isolated polypeptide of claim 11, wherein the immunoglobulin protein is selected an IgG, an IgM, an IgA, an IgD or, an IgE.

13. The isolated polypeptide of claim 12, wherein the immunoglobulin protein is an IgE.

14. The isolated polypeptide of claim 12, wherein the immunoglobulin is an IgG.

15. The isolated polypeptide of claim 14, wherein the IgG is an IgG1, an IgG2, an IgG3, or an IgG4.

16. The isolated polypeptide of claim 11, wherein the portion of the immunoglobulin protein is an Fc region, an Fab region, a heavy chain variable (VH) domain, a heavy chain constant domain, a light chain variable (VL) domain, or a light chain constant domain.

17. The isolated polypeptide of claim 14, wherein the portion of the immunoglobulin protein is an Fc region.

18. The isolated polypeptide of claim 14, wherein the Fc region is a human Fc region and where in the human Fc region is a human IgG1 Fc region (SEQ ID NO: 5), a human IgG2 Fc region (SEQ ID NO: 7), a human IgG3 Fc region (SEQ ID NO: 9), or a human IgG4 Fc region (SEQ ID NO: 11).

19. The isolated polypeptide of claim 14, wherein the Fc region is a canine Fc region and wherein the canine Fc region is a canine IgG heavy chain D Fc region (SEQ ID NO: 20), a canine IgG heavy chain A Fc region (SEQ ID NO: 22), a canine IgG heavy chain B Fc region (SEQ ID NO: 24), or a canine IgG heavy chain C Fc region (SEQ ID NO: 26).

20. The isolated polypeptide of claim 16, wherein the portion of the immunoglobulin protein is a heavy chain constant domain.

21. The isolated polypeptide of claim 20, wherein the heavy chain constant is a human heavy chain constant region and wherein the human heavy chain constant region is a human IgG1 heavy chain constant domain (SEQ ID NO: 4), a human IgG2 heavy chain constant domain (SEQ ID NO: 6), a human IgG3 heavy chain constant domain (SEQ ID NO: 8), or a human IgG4 heavy chain constant domain (SEQ ID NO: 10).

22. The isolated polypeptide of claim 20, wherein the heavy chain constant is a canine heavy chain constant region and wherein the canine heavy chain constant region is a canine IgG heavy chain constant domain chain D (SEQ ID NO: 19), a canine IgG heavy chain constant domain chain A (SEQ ID NO: 21), a canine IgG heavy chain constant domain chain B (SEQ ID NO: 23), or a canine IgG heavy chain constant domain chain C (SEQ ID NO: 25).

23. The isolated polypeptide of claim 1, wherein the DSG2 fusion polypeptide further comprises a linker.

24. The isolated polypeptide of claim 23, wherein the linker is from about 5 amino acids to about 50 amino acids in length.

25. The isolated polypeptide of claim 24, wherein the linker is GGGGGS (SEQ ID NO: 12), EAAAK (SEQ ID NO: 13), GGGGS (SEQ ID NO: 27) or IEGRMD (SEQ ID NO: 28).

26. The isolated polypeptide of claim 1, wherein the DSG2 fusion polypeptide further comprises an affinity tag.

27. The isolated polypeptide of claim 10, wherein the whole extracellular region of DSG2 protein comprises the amino acid sequence of SEQ ID NO: 3.

28. The isolated polypeptide of claim 9, wherein the portion of the DSG2 protein is a portion of an extracellular region of the DSG2 protein.

29. The isolated polypeptide of claim 1, wherein the portion of an extracellular region of the DSG2 protein comprises at least one domain selected from the group consisting of extracellular cadherin domain 1 (EC1), extracellular cadherin domain 2 (EC2), extracellular cadherin domain 3 (EC3), extracellular cadherin domain 4 (EC4), and extracellular anchor domain (EA).

30. A cell expressing the isolated polypeptide of claim 1.

31. A method of reducing a proarrhythmic phenotype in a cardiomyocyte, the method comprising: contacting the cardiomyocyte with the DSG2 fusion polypeptides of claim 1.

32. The method of claim 31, wherein the proarrhythmic phenotype is associated with anti-DSG2 antibodies.

33. A method of improving cardiomyocyte electrical function, the method comprising:
contacting the cardiomyocyte with DSG2 fusion polypeptide of claim 1, and
measuring cardiomyocyte sodium spike (u V/m), wherein increase in the sodium spike after contacting with the DSG2 fusion polypeptides is indicative of improved cardiomyocyte electrical function.

34. The method of claim 33, wherein the cardiomyocyte is pre-exposed to anti-DSG2 antibodies.

35. A method of treating a condition associated with serum anti DSG2 autoantibodies, the method comprising contacting the subject with the isolated polypeptide of claim 1.

36. A method of treating arrhythmia and/or cardiomyopathy in a subject, the method comprising:
contacting the subject with the isolated polypeptide of claim 1, and
measuring one or more symptoms associated with arrhythmia selected from the group consisting of palpitations, dizziness, lightheadedness, syncope, heart failure and reduced ejection fraction, and/or
measuring one or more symptoms associated with cardiomyopathy selected from the group consisting of arrhythmia, palpitations, lightheadedness, dizziness, syncope, myocarditis, heart failure, poor cardiac output, and reduced ejection fraction.

37. The method of claim 36, wherein the arrhythmia and/or cardiomyopathy is arrhythmogenic right ventricular cardiomyopathy.

38. The method of claim 36, wherein the arrhythmia and/or cardiomyopathy is associated with sarcoidosis.

39. The method of claim 36, wherein the arrhythmia and/or cardiomyopathy is dilated cardiomyopathy.

40. The method of claim 36, wherein the arrhythmia and/or cardiomyopathy is associated with serum anti-DSG2 antibodies.

41. A method of treating a cardiac abnormality in a subject, the method comprising contacting the subject with the isolated polypeptide of claim 1, wherein the serum of the subject comprises anti-DSG2 antibodies.

42. A method of reducing anti-DSG2 antibodies in a subject, the method comprising:
(i) contacting the subject with the isolated polypeptide of claim 1, and
(ii) measuring the level of anti-DSG2 antibodies in the subject, wherein contacting the subject with the isolated polypeptides reduces the level of the anti-DSG2 antibodies.

43. The method of claim 42, wherein the anti-DSG2 antibodies is reduced by about 1%, 5%, 10%, 15%, 20%, 25%, 30%, 35%, 40%, 45%, 50%, 55%, 60%, 65%, 70%, 75%, 80%, 85%, 90%, 95%, or 100%.

44. A composition comprising the isolated polypeptide of claim 1 and at least one therapeutic agent.

45. The composition of claim 44, wherein the at least one therapeutic agent is an anti-CD20 antibody.

46. The composition of claim 44, wherein the at least one therapeutic agent is an FcRn-blocking antibody.

47. The composition of claim 44, wherein the at least one therapeutic agent is intravenous immunoglobulins (IVIG).

48. The composition of claim 44, wherein the at least one therapeutic agent is eculizumab.

49. A method of treating a condition associated with serum anti DSG2 autoantibodies, the method comprising contacting the subject with the isolated polypeptide of claim 1, and at least one therapeutic agent or a combination thereof.

50. The method of claim 49, wherein the subject is contacted with the at least one therapeutic agent.

51. The method of claim 50, wherein the at least one therapeutic agent is an anti-CD20 antibody.

52. The method of claim 50, wherein the at least one therapeutic agent is an FcRn-blocking antibody.

53. The method of claim 50, wherein the at least one therapeutic agent is intravenous immunoglobulins (IVIG).

54. The method of claim 50, wherein the at least one therapeutic agent is eculizumab.

55. A pharmaceutical composition for use in treatment of a disease or disorder caused by anti-desmoglein-2 (DSG2) autoantibodies, the composition comprising:
a fusion polypeptide which blocks binding of anti-DSG2 antibodies to the extracellular domain of DSG2, the fusion polypeptide comprising:
an extracellular region of a human DSG2 protein having at least about 85% sequence identity with amino acid residues 50 to 609 of SEQ ID NO: 1, or a portion thereof, and an affinity tag.

56. The pharmaceutical composition of claim 55, wherein the portion of the extracellular region of the human DSG2 protein includes any one of or a combination of DSG2 domains selected from the group consisting of: extracellular cadherin domain 1 (EC1), extracellular cadherin domain 2 (EC2), extracellular cadherin domain 3 (EC3), extracellular cadherin domain 4 (EC4), and extracellular anchor domain (EA).

57. The pharmaceutical composition of claim 55, wherein the affinity tag is an Fc region of an immunoglobulin.

58. The pharmaceutical composition of claim 57, wherein the immunoglobulin is IgG1 or IgG4.

59. The pharmaceutical composition of claim 57, wherein the immunoglobulin is a variant of IgG1 having the sequence of SEQ ID NO: 31.

60. The pharmaceutical composition of claim 57, wherein the immunoglobulin is a variant of IgG4 having the sequence of SEQ ID NO: 32.

61. The pharmaceutical composition of claim 55, wherein the affinity tag is a polyhistidine tag.

62. The pharmaceutical composition of claim 55, further comprising a linker sequence located between the extracellular region of a human DSG2 protein or portion thereof, and the affinity tag.

63. The pharmaceutical composition of claim 62, wherein the linker sequence is SEQ ID NO: 12, 13, 27 or 28.

64. The pharmaceutical composition of claim 55, wherein the disease or disorder is arrhythmia and/or cardiomyopathy.

65. The pharmaceutical composition of claim 64, wherein the arrhythmia and/or cardiomyopathy is arrhythmogenic right ventricular cardiomyopathy (ARVC), sarcoidosis, post-acute sequelae of COVID-19, or dilated cardiomyopathy.

66. The pharmaceutical composition of claim 64, wherein the arrhythmia and/or cardiomyopathy is caused proximally or distally by a virus.

67. The pharmaceutical composition of claim 66, wherein the virus is SARS-COV2, adenovirus, hepatitis virus, hepatitis C virus, parvovirus, herpes simplex virus, echovirus, Epstein-Barr virus, rubella, cytomegalovirus, or HIV.
